SEQUENCE LISTING (167 sequences)

SEQ ID NO: 1 - protein, 9 aa, Artificial Sequence
  VH-CDR1 Sequence
YTFTSYYIH

SEQ ID NO: 2 - protein, 17 aa, Artificial Sequence
  VH-CDR2 Sequence
IINPSGGSTSYAQKFQG

SEQ ID NO: 3 - protein, 13 aa, Artificial Sequence
  VH-CDR3 Sequence
ARGTMVSRPSLDL

SEQ ID NO: 4 - protein, 9 aa, Artificial Sequence
  VH-CDR1 Sequence
YTFRRYYMH

SEQ ID NO: 5 - protein, 17 aa, Artificial Sequence
  VH-CDR2 Sequence
IINPHVGSTSYAQKFQG

SEQ ID NO: 6 - protein, 16 aa, Artificial Sequence
  VH-CDR3 Sequence
ARGPDTAIHQGPAFDI

SEQ ID NO: 7 - protein, 9 aa, Artificial Sequence
  VH-CDR1 Sequence
YTFTSYYMH

SEQ ID NO: 8 - protein, 17 aa, Artificial Sequence
  VH-CDR2 Sequence
IINPSGGSTSYAQKFQG

SEQ ID NO: 9 - protein, 13 aa, Artificial Sequence
  VH-CDR3 Sequence
ARQGAGGTMHFDY

SEQ ID NO: 10 - protein, 9 aa, Artificial Sequence
  VH-CDR1 Sequence
YTFQAYYMH

SEQ ID NO: 11 - protein, 17 aa, Artificial Sequence
  VH-CDR2 Sequence
IINPSNGHTSYAQKFQG

SEQ ID NO: 12 - protein, 13 aa, Artificial Sequence
  VH-CDR3 Sequence
ARQGAGGTMHFDY

SEQ ID NO: 13 - protein, 9 aa, Artificial Sequence
  VH-CDR1 Sequence
YTFPSYYMH

SEQ ID NO: 14 - protein, 17 aa, Artificial Sequence
  VH-CDR2 Sequence
IINPSGGNTHYAQKFQG

SEQ ID NO: 15 - protein, 13 aa, Artificial Sequence
  VH-CDR3 Sequence
ARQGAGGTMHFDY

SEQ ID NO: 16 - protein, 9 aa, Artificial Sequence
  VH-CDR1 Sequence
YTFDSLYMH

SEQ ID NO: 17 - protein, 17 aa, Artificial Sequence
  VH-CDR2 Sequence
IINPSNGRTSYAQKFQG

SEQ ID NO: 18 - protein, 13 aa, Artificial Sequence
  VH-CDR3 Sequence
ARQGAGGTMHFDY

SEQ ID NO: 19 - protein, 9 aa, Artificial Sequence
  VH-CDR1 Sequence
YTFEEYYMH

SEQ ID NO: 20 - protein, 17 aa, Artificial Sequence
  VH-CDR2 Sequence
IINPSNGRTSYAQKFQG

SEQ ID NO: 21 - protein, 13 aa, Artificial Sequence
  VH-CDR3 Sequence
ARQGAGGTMHFDY

SEQ ID NO: 22 - protein, 9 aa, Artificial Sequence
  VH-CDR1 Sequence
YTFTRYYMH

SEQ ID NO: 23 - protein, 17 aa, Artificial Sequence
  VH-CDR2 Sequence
WINPNSGGTNYAQKFQG

SEQ ID NO: 24 - protein, 12 aa, Artificial Sequence
  VH-CDR3 Sequence
ARDPPMGQGMDV

SEQ ID NO: 25 - protein, 9 aa, Artificial Sequence
  VH-CDR1 Sequence
YTFTRYYMH

SEQ ID NO: 26 - protein, 17 aa, Artificial Sequence
  VH-CDR2 Sequence
WINPNSDGTNYAQKFQG

SEQ ID NO: 27 - protein, 12 aa, Artificial Sequence
  VH-CDR3 Sequence
ARDPPMGQGMDV

SEQ ID NO: 28 - protein, 9 aa, Artificial Sequence
  VH-CDR1 Sequence
YTFESYYMH

SEQ ID NO: 29 - protein, 17 aa, Artificial Sequence
  VH-CDR2 Sequence
WINPPSGRTNYAQKFQG

SEQ ID NO: 30 - protein, 12 aa, Artificial Sequence
  VH-CDR3 Sequence
ARDPPMGQGMDV

SEQ ID NO: 31 - protein, 9 aa, Artificial Sequence
  VH-CDR1 Sequence
YTFTRYYMH

SEQ ID NO: 32 - protein, 17 aa, Artificial Sequence
  VH-CDR2 Sequence
WINPPSGGTNYAQKFQG

SEQ ID NO: 33 - protein, 12 aa, Artificial Sequence
  VH-CDR3 Sequence
ARDPPMGQGMDV

SEQ ID NO: 34 - protein, 9 aa, Artificial Sequence
  VH-CDR1 Sequence
YTFTSRYMH

SEQ ID NO: 35 - protein, 17 aa, Artificial Sequence
  VH-CDR2 Sequence
IINPRLGSTSYAQKFQG

SEQ ID NO: 36 - protein, 16 aa, Artificial Sequence
  VH-CDR3 Sequence
ARDGEVGARDLGWFDP

SEQ ID NO: 37 - protein, 9 aa, Artificial Sequence
  VH-CDR1 Sequence
YTFRSRYMH

SEQ ID NO: 38 - protein, 17 aa, Artificial Sequence
  VH-CDR2 Sequence
IINPMYGSTSYAQKFQG

SEQ ID NO: 39 - protein, 16 aa, Artificial Sequence
  VH-CDR3 Sequence
ARDGEVGARDLGWFDP

SEQ ID NO: 40 - protein, 9 aa, Artificial Sequence
  VH-CDR1 Sequence
YTFGSHYMH

SEQ ID NO: 41 - protein, 17 aa, Artificial Sequence
  VH-CDR2 Sequence
IINPSIGSTSYAQKFQG

SEQ ID NO: 42 - protein, 16 aa, Artificial Sequence
  VH-CDR3 Sequence
ARDGEVGARDLGWFDP

SEQ ID NO: 43 - protein, 9 aa, Artificial Sequence
  VH-CDR1 Sequence
YTFSHYYMH

SEQ ID NO: 44 - protein, 17 aa, Artificial Sequence
  VH-CDR2 Sequence
IINPRYGSTSYAQKFQG

SEQ ID NO: 45 - protein, 16 aa, Artificial Sequence
  VH-CDR3 Sequence
ARDGEVGARDLGWFDP

SEQ ID NO: 46 - protein, 9 aa, Artificial Sequence
  VH-CDR1 Sequence
YTFPSRYMH

SEQ ID NO: 47 - protein, 17 aa, Artificial Sequence
  VH-CDR2 Sequence
IINPSLGSTSYAQKFQG

SEQ ID NO: 48 - protein, 16 aa, Artificial Sequence
  VH-CDR3 Sequence
ARDGEVGARDLGWFDP

SEQ ID NO: 49 - protein, 9 aa, Artificial Sequence
  VH-CDR1 Sequence
YTFALYYMH

SEQ ID NO: 50 - protein, 17 aa, Artificial Sequence
  VH-CDR2 Sequence
IINPHVGSTSYAQKFQG

SEQ ID NO: 51 - protein, 17 aa, Artificial Sequence
  VH-CDR3 Sequence
ARGRPLSGTGGHHYFDY

SEQ ID NO: 52 - protein, 9 aa, Artificial Sequence
  VH-CDR1 Sequence
YTFMRYYMH

SEQ ID NO: 53 - protein, 17 aa, Artificial Sequence
  VH-CDR2 Sequence
IINPGLGSTSYAQKFQG

SEQ ID NO: 54 - protein, 17 aa, Artificial Sequence
  VH-CDR3 Sequence
ARGRPLSGTGGHHYFDY

SEQ ID NO: 55 - protein, 9 aa, Artificial Sequence
  VH-CDR1 Sequence
YTFGEYYMH

SEQ ID NO: 56 - protein, 17 aa, Artificial Sequence
  VH-CDR2 Sequence
IINPRFGSTSYAQKFQG

SEQ ID NO: 57 - protein, 17 aa, Artificial Sequence
  VH-CDR3 Sequence
ARGRPLSGTGGHHYFDY

SEQ ID NO: 58 - protein, 9 aa, Artificial Sequence
  VH-CDR1 Sequence
YTFTSYYMH

SEQ ID NO: 59 - protein, 17 aa, Artificial Sequence
  VH-CDR2 Sequence
IINPRVGSTSYAQKFQG

SEQ ID NO: 60 - protein, 17 aa, Artificial Sequence
  VH-CDR3 Sequence
ARGRPLSGTGGHHYFDY

SEQ ID NO: 61 - protein, 11 aa, Artificial Sequence
  VL-CDR4 Sequence
RASQSVSSSLA

SEQ ID NO: 62 - protein, 7 aa, Artificial Sequence
  VL-CDR5 Sequence
GASTRAT

SEQ ID NO: 63 - protein, 9 aa, Artificial Sequence
  VL-CDR6 Sequence
QQDNVWPIT

SEQ ID NO: 64 - protein, 11 aa, Artificial Sequence
  VL-CDR4 Sequence
RASQSISSYLN

SEQ ID NO: 65 - protein, 7 aa, Artificial Sequence
  VL-CDR5 Sequence
AASSLQS

SEQ ID NO: 66 - protein, 10 aa, Artificial Sequence
  VL-CDR6 Sequence
QQDAVIPPFT

SEQ ID NO: 67 - protein, 11 aa, Artificial Sequence
  VL-CDR4 Sequence
RASQSVSSYLA

SEQ ID NO: 68 - protein, 7 aa, Artificial Sequence
  VL-CDR5 Sequence
DASNRAT

SEQ ID NO: 69 - protein, 8 aa, Artificial Sequence
  VL-CDR6 Sequence
QQRAPFFT

SEQ ID NO: 70 - protein, 11 aa, Artificial Sequence
  VL-CDR4 Sequence
RASQSVSSYLA

SEQ ID NO: 71 - protein, 7 aa, Artificial Sequence
  VL-CDR5 Sequence
DASNRAT

SEQ ID NO: 72 - protein, 8 aa, Artificial Sequence
  VL-CDR6 Sequence
QQRAPFFT

SEQ ID NO: 73 - protein, 11 aa, Artificial Sequence
  VL-CDR4 Sequence
RASQSVSSYLA

SEQ ID NO: 74 - protein, 7 aa, Artificial Sequence
  VL-CDR5 Sequence
DASNRAT

SEQ ID NO: 75 - protein, 8 aa, Artificial Sequence
  VL-CDR6 Sequence
QQRAPFFT

SEQ ID NO: 76 - protein, 11 aa, Artificial Sequence
  VL-CDR4 Sequence
RASQSVSSYLA

SEQ ID NO: 77 - protein, 7 aa, Artificial Sequence
  VL-CDR5 Sequence
DASNRAT

SEQ ID NO: 78 - protein, 8 aa, Artificial Sequence
  VL-CDR6 Sequence
QQRAPFFT

SEQ ID NO: 79 - protein, 11 aa, Artificial Sequence
  VL-CDR4 Sequence
RASQSVSSYLA

SEQ ID NO: 80 - protein, 7 aa, Artificial Sequence
  VL-CDR5 Sequence
DASNRAT

SEQ ID NO: 81 - protein, 8 aa, Artificial Sequence
  VL-CDR6 Sequence
QQRAPFFT

SEQ ID NO: 82 - protein, 11 aa, Artificial Sequence
  VL-CDR4 Sequence
RASQSISSYLN

SEQ ID NO: 83 - protein, 7 aa, Artificial Sequence
  VL-CDR5 Sequence
AASSLQS

SEQ ID NO: 84 - protein, 9 aa, Artificial Sequence
  VL-CDR6 Sequence
QQGFSSPFT

SEQ ID NO: 85 - protein, 11 aa, Artificial Sequence
  VL-CDR4 Sequence
RASQSISSYLN

SEQ ID NO: 86 - protein, 7 aa, Artificial Sequence
  VL-CDR5 Sequence
AASSLQS

SEQ ID NO: 87 - protein, 9 aa, Artificial Sequence
  VL-CDR6 Sequence
QQSFVAPFT

SEQ ID NO: 88 - protein, 11 aa, Artificial Sequence
  VL-CDR4 Sequence
RASQSISSYLN

SEQ ID NO: 89 - protein, 7 aa, Artificial Sequence
  VL-CDR5 Sequence
AASSLQS

SEQ ID NO: 90 - protein, 9 aa, Artificial Sequence
  VL-CDR6 Sequence
QQSFVAPFT

SEQ ID NO: 91 - protein, 11 aa, Artificial Sequence
  VL-CDR4 Sequence
RASQSISSYLN

SEQ ID NO: 92 - protein, 7 aa, Artificial Sequence
  VL-CDR5 Sequence
AASSLQS

SEQ ID NO: 93 - protein, 9 aa, Artificial Sequence
  VL-CDR6 Sequence
QQSFVAPFT

SEQ ID NO: 94 - protein, 11 aa, Artificial Sequence
  VL-CDR4 Sequence
RASQSVSSNLA

SEQ ID NO: 95 - protein, 7 aa, Artificial Sequence
  VL-CDR5 Sequence
GASTRAT

SEQ ID NO: 96 - protein, 9 aa, Artificial Sequence
  VL-CDR6 Sequence
QQYTPLPWT

SEQ ID NO: 97 - protein, 11 aa, Artificial Sequence
  VL-CDR4 Sequence
RASQSVSSNLA

SEQ ID NO: 98 - protein, 7 aa, Artificial Sequence
  VL-CDR5 Sequence
GASTRAT

SEQ ID NO: 99 - protein, 9 aa, Artificial Sequence
  VL-CDR6 Sequence
QQYTPLPWT

SEQ ID NO: 100 - protein, 11 aa, Artificial Sequence
  VL-CDR4 Sequence
RASQSVSSNLA

SEQ ID NO: 101 - protein, 7 aa, Artificial Sequence
  VL-CDR5 Sequence
GASTRAT

SEQ ID NO: 102 - protein, 9 aa, Artificial Sequence
  VL-CDR6 Sequence
QQYTPLPWT

SEQ ID NO: 103 - protein, 11 aa, Artificial Sequence
  VL-CDR4 Sequence
RASQSVSSNLA

SEQ ID NO: 104 - protein, 7 aa, Artificial Sequence
  VL-CDR5 Sequence
GASTRAT

SEQ ID NO: 105 - protein, 9 aa, Artificial Sequence
  VL-CDR6 Sequence
QQYTPLPWT

SEQ ID NO: 106 - protein, 11 aa, Artificial Sequence
  VL-CDR4 Sequence
RASQSVSSNLA

SEQ ID NO: 107 - protein, 7 aa, Artificial Sequence
  VL-CDR5 Sequence
GASTRAT

SEQ ID NO: 108 - protein, 9 aa, Artificial Sequence
  VL-CDR6 Sequence
QQYTPLPWT

SEQ ID NO: 109 - protein, 11 aa, Artificial Sequence
  VL-CDR4 Sequence
QASQDISNYLN

SEQ ID NO: 110 - protein, 7 aa, Artificial Sequence
  VL-CDR5 Sequence
DASNLET

SEQ ID NO: 111 - protein, 10 aa, Artificial Sequence
  VL-CDR6 Sequence
QQVYALPPWT

SEQ ID NO: 112 - protein, 11 aa, Artificial Sequence
  VL-CDR4 Sequence
QASQDISNYLN

SEQ ID NO: 113 - protein, 7 aa, Artificial Sequence
  VL-CDR5 Sequence
DASNLET

SEQ ID NO: 114 - protein, 10 aa, Artificial Sequence
  VL-CDR6 Sequence
QQVYALPPWT

SEQ ID NO: 115 - protein, 11 aa, Artificial Sequence
  VL-CDR4 Sequence
QASQDISNYLN

SEQ ID NO: 116 - protein, 7 aa, Artificial Sequence
  VL-CDR5 Sequence
DASNLET

SEQ ID NO: 117 - protein, 10 aa, Artificial Sequence
  VL-CDR6 Sequence
QQVYALPPWT

SEQ ID NO: 118 - protein, 11 aa, Artificial Sequence
  VL-CDR4 Sequence
QASQDISNYLN

SEQ ID NO: 119 - protein, 7 aa, Artificial Sequence
  VL-CDR5 Sequence
DASNLET

SEQ ID NO: 120 - protein, 10 aa, Artificial Sequence
  VL-CDR6 Sequence
QQVYALPPWT

SEQ ID NO: 121 - protein, 504 aa, Staphylococcus aureus
MMTLQIHTGGINLKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDNNKPGKEDNNKPGKEDGNKPGKEDNKKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 122 - protein, 492 aa, Staphylococcus aureus
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDGNKPGKEDGNKPGKEDNNKPGKEDGNKPGKEDNKKPGKEDGNKPGKEDNNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 123 - protein, 520 aa, Staphylococcus aureus
MMTLQIHTGGINLKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNKKPGKEDGNKPGKEDGNKPGKEDNKKPGKEDGNKPGKEDNNKPGKEDGNKPGKEDNNKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 124 - protein, 520 aa, Staphylococcus aureus
MMTLQIHTGGINLKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDNNKPGKEDNNKPGKEDGNKPGKEDNKKPGKEDGNKPGKEDNKKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 125 - protein, 520 aa, Staphylococcus aureus; identical to SEQ ID NO: 123

SEQ ID NO: 126 - protein, 492 aa, Staphylococcus aureus; identical to SEQ ID NO: 122

SEQ ID NO: 127 - protein, 516 aa, Staphylococcus aureus
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNKFNKDQQSAFYEILNMPNLNEEQRNGFIQSLKDDPSQSTNVLGEAKKLNESQAPKADNNFNKEQQNAFYEILNMPNLNEEQRNGFIQSLKDDPSQSANLLAEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDGNKPGKEDNKKPGKEDGNKPGKEDNKKPGKEDGNKPGKEDGNKPGKEDGNKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 128 - protein, 450 aa, Staphylococcus aureus
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSVSKEILAEAKKLNDAQAPKEEDNKKPGKEDGNKPGKEDGNKPGKEDNKKPGKEDGNKPGKEDNNKPGKEDGNKPGKEDNNKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 129 - protein, 450 aa, Staphylococcus aureus; identical to SEQ ID NO: 128

SEQ ID NO: 130 - protein, 450 aa, Staphylococcus aureus; identical to SEQ ID NO: 128

SEQ ID NO: 131 - protein, 516 aa, Staphylococcus aureus
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDNNKPGKEDNNKPGKEDNNKPGKEDGNKPGKEDNKKPGKEDGNKPGKEDNKKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 132 - protein, 508 aa, Staphylococcus aureus
MKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDNNKPGKEDNNKPGKEDGNKPGKEDNKKPGKEDGNKPGKEDNKKPGKEDGNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL

SEQ ID NO: 133 - protein, 508 aa, Staphylococcus aureus; identical to SEQ ID NO: 132

SEQ ID NO: 134 - protein, 436 aa, Staphylococcus aureus [sequence omitted]

SEQ ID NO: 135 - protein, 61 aa, Staphylococcus aureus
ADNNFNKEQQNAFYEILNMPNLNEEQRNGFIQSLKDDPSQSANLLSEAKKLNESQAPKGGC

SEQ ID NO: 136 - protein, 61 aa, Staphylococcus aureus
ADNKFNKEQQNAFYEILHLPNLNEEQRNGFIQSLKDDPSQSANLLAEAKKLNDAQAPKGGC

SEQ ID NO: 137 - protein, 61 aa, Staphylococcus aureus
ADNKFNKEQQNAFYEILHLPNLTEEQRNGFIQSLKDDPSVSKEILAEAKKLNDAQAPKGGC

SEQ ID NO: 138 - protein, 64 aa, Staphylococcus aureus
ADAQQNNFNKDQQSAFYEILNMPNLNEAQRNGFIQSLKDDPSQSTNVLGEAKKLNESQAPKGGC

SEQ ID NO: 139 - protein, 59 aa, Staphylococcus aureus
AQHDEAQQNAFYQVLNMPNLNADQRNGFIQSLKDDPSQSANVLGEAQKLNDSQAPKGGC

SEQ ID NO: 140 - protein, 70 aa, Staphylococcus aureus
GSENTQQTSTKHQTTQNNYVTDQQKAFYQVLHLKGITEEQRNQYIKTLREHPERAQEVFSESLKDSKGGC

SEQ ID NO: 141 - protein, 64 aa, Staphylococcus aureus
GNPDRRVAQQNAFYNVLKNDNLTEQEKNNYIAQIKENPDRSQQVWVESVQSSKAKERQNIEGGC

SEQ ID NO: 142 - protein, 59 aa, Staphylococcus aureus
AQHDEAKKNAFYQVLNMPNLNADQRNGFIQSLKAAPSQSANVLGEAQKLNDSQAPKGGC

SEQ ID NO: 143 - protein, 64 aa, Artificial Sequence
  SpA-DKKAA mutant
ADAQQNNFNKDKKSAFYEILNMPNLNEAQRNGFIQSLKAAPSQSTNVLGEAKKLNESQAPKGGC

SEQ ID NO: 144 - protein, 64 aa, Artificial Sequence
  SpA-DKK mutant
ADAQQNNFNKDKKSAFYEILNMPNLNEAQRNGFIQSLKDDPSQSTNVLGEAKKLNESQAPKGGC

SEQ ID NO: 145 - protein, 9 aa, Artificial Sequence
  SpA consensus sequence
  MUTAGEN 3: wherein X is any of N, S or K
  MUTAGEN 7: wherein X is any of E, Q or N
  MUTAGEN 8: wherein X is any of I or V
QQXAFYXXL

SEQ ID NO: 146 - protein, 16 aa, Artificial Sequence
  SpA consensus sequence
  MUTAGEN 15: wherein X is any of Q or V
QRNGFIQSLKDDPSXS

SEQ ID NO: 147 - protein, 9 aa, Artificial Sequence
  IGBP mutant consensus sequence
  MUTAGEN 3: wherein X is any of N, S or K
  MUTAGEN 7: wherein X is any of E, Q or N
  MUTAGEN 8: wherein X is any of I or V
KKXAFYXXL

SEQ ID NO: 148 - protein, 16 aa, Artificial Sequence
  IGBP mutant consensus sequence
  MUTAGEN 15: wherein X is any of Q or V
QRNGFIQSLKAAPSXS

SEQ ID NO: 149 - protein, 9 aa, Artificial Sequence
  VH-CDR1
YTFTGRYMH

SEQ ID NO: 150 - protein, 17 aa, Artificial Sequence
  VH-CDR2
IINPRLGSTSYAQKFQG

SEQ ID NO: 151 - protein, 16 aa, Artificial Sequence
  VH-CDR3
ARDGEVGARDLGWFDP

SEQ ID NO: 152 - protein, 472 aa, Artificial Sequence
  Heavy Chain
MELGLCWVFLVAILEGVQCQVQLVQSGAEVKKPGASVKVSCKASGYTFTGRYMHWVRQAPGQGLEWMGIINPRLGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 471 aa, Artificial Sequence
  Heavy Chain
MELGLCWVFLVAILEGVQCEVQLVQSGAEVKKPGASVKVSCKASGYTFTGRYMHWVRQAPGQGLEWMGIINPRLGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 154 - protein, 472 aa, Artificial Sequence
  Heavy Chain
MELGLCWVFLVAILEGVQCQVQLVQSGAEVKKPGASVKVSCKASGYTFTSRYMHWVRQAPGQGLEWMGIINPRLGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 471 aa, Artificial Sequence
  Heavy Chain
MELGLCWVFLVAILEGVQCEVQLVQSGAEVKKPGASVKVSCKASGYTFTSRYMHWVRQAPGQGLEWMGIINPRLGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 156 - protein, 472 aa, Artificial Sequence
  Heavy Chain
MELGLCWVFLVAILEGVQCQVQLVQSGAEVKKPGASVKVSCKASGYTFRSRYMHWVRQAPGQGLEWMGIINPMYGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 471 aa, Artificial Sequence
  Heavy Chain
MELGLCWVFLVAILEGVQCEVQLVQSGAEVKKPGASVKVSCKASGYTFRSRYMHWVRQAPGQGLEWMGIINPMYGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 158 - protein, 468 aa, Artificial Sequence
  Heavy Chain
MELGLCWVFLVAILEGVQCQVQLQESGPGLVKPSETLSLTCAVSGYSISSGMGWGWIRQPPGKGLEWIGSIDQRGSTYYNPSLKSRVTISVDTSKNQFSLKLSSVTAADTAVYYCARDAGHGVDMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 471 aa, Artificial Sequence
  Heavy Chain
MELGLCWVFLVAILEGVQCEVQLVQSGAEVKKPGASVKVSCKASGYTFSHYYMHWVRQAPGQGLEWMGIINPRYGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 160 - protein, 471 aa, Artificial Sequence
  Heavy Chain
MELGLCWVFLVAILEGVQCEVQLVQSGAEVKKPGASVKVSCKASGYTFSHYYMHWVRQAPGQGLEWMGIINPRYGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVQFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPG

SEQ ID NO: 161 - protein, 471 aa, Artificial Sequence
  Heavy Chain
MELGLCWVFLVAILEGVQCEVQLVQSGAEVKKPGASVKVSCKASGYTFSHYYMHWVRQAPGQGLEWMGIINPRYGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPG

SEQ ID NO: 162 - protein, 471 aa, Artificial Sequence
  Heavy Chain
MELGLCWVFLVAILEGVQCEVQLVQSGAEVKKPGASVKVSCKASGYTFSHYYMHWVRQAPGQGLEWMGIINPRYGSTSYAQKFQGRVTMTRDTSTSTVYMELSSLRSEDTAVYYCARDGEVGARDLGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRYTQKSLSLSPG

SEQ ID NO: 163 - protein, 233 aa, Artificial Sequence
  Light Chain
MELGLCWVFLVAILEGVQCEIVLTQSPATLSVSPGERATLSCRASQSVSSNLAWYQQKPGQAPRLLIYGASTRATGIPARFSGSGSGTEFTLTISSLQSEDFAVYYCQQYTPLPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 9 aa, Artificial Sequence
  VH CDR1
  MUTAGEN 4: wherein X is any of T, R, Q, P, D, E, G, S, A, M
  MUTAGEN 5: wherein X is any of S, R, A, E, H, L, G
  MUTAGEN 6: wherein X is any of Y, L, R, H
  MUTAGEN 8: wherein X is any of I, M
YTFXXXYXH

SEQ ID NO: 165 - protein, 17 aa, Artificial Sequence
  MUTAGEN 1: wherein X is any of I, W
  VH CDR2
  MUTAGEN 5: wherein X is any of S, H, N, P, R, M, G
  MUTAGEN 6: wherein X is any of G, V, N, S, L, Y, I, V, F
  MUTAGEN 7: wherein X is any of G, D
  MUTAGEN 8: wherein X is any of S, H, N, R, G
  MUTAGEN 10: wherein X is any of S, H, N
XINPXXXXTXYAQKFQG

SEQ ID NO: 166 - protein, 11 aa, Artificial Sequence
  MUTAGEN 1: wherein X is any of R, Q
  VL CDR1
  MUTAGEN 5: wherein X is any of S, D
  MUTAGEN 6: wherein X is any of V, I
  MUTAGEN 8: wherein X is any of S, N
  MUTAGEN 9: wherein X is any of S, Y, N
  MUTAGEN 11: wherein X is any of A, N
XASQXXSXXLX

SEQ ID NO: 167 - protein, 7 aa, Artificial Sequence
  MUTAGEN 1: wherein X is any of G, A, D
  VL CDR2
  MUTAGEN 4: wherein X is any T, S, N
  MUTAGEN 5: wherein X is any R, L
  MUTAGEN 6: wherein X is any A, Q, E
  MUTAGEN 7: wherein X is any T, S
XASXXXX

(19) United States
(12) Patent Application Publication
(10) Pub. No.: US 2018/0105584 A1
(43) Pub. Date: Apr. 19, 2018

(54) ANTIBODY DIRECTED AGAINST IMMUNOGLOBULIN-BINDING PROTEINS OF S. AUREUS

(71) Applicant: ARSANIS BIOSCIENCES GMBH, Vienna (AT)

(72) Inventors: Eszter NAGY, Vienna (AT); Adriana BADARAU, Vienna (AT); Harald ROUHA, Wien (AT); Lukas STULIK, Vienna (AT); Irina MIRKINA, Vienna (AT); Michael B. BATTLES, Lebanon, NH (US); Nels NIELSON, Lebanon, NH (US)

(21) Appl. No.: 15/567,337

(22) PCT Filed: Apr. 14, 2016
(86) PCT No.: PCT/EP2016/058238

§ 371 Date: Oct. 17, 2017

(30) Foreign Application Priority Data
Apr. 17, 2015 (EP) 15163996.0

(51) Int. Cl. C07K 16/12; A61K 39/40

(52) U.S. Cl. CPC C07K 16/1271; A61K 39/40; C07K 2317/76; A61K 2039/505; C07K 2317/55; C07K 2317/92; C07K 2317/565; C07K 2319/01

(57) ABSTRACT

A monoclonal antibody that counteracts Staphylococcus aureus by specifically binding to wild-type immunoglobulin-binding proteins (IGBP) of S. aureus comprising a cross-specific CDR binding site recognizing at least three of the IGBP domains selected from the group consisting of Protein A (SpA) domains and immunoglobulin-binding protein (Sbi) domains SpA-A, SpA-B, SpA-C, SpA-D, SpA-E, Sbi-I, and Sbi-II, wherein the antibody has an affinity to bind SpA-E with a K_{D} of less than 5×10⁻⁹M as determined by a standard optical interferometry method for a F(ab)2 fragment, and preferably binds to wt SpA equally or better compared to mutant SpA-KKAA that lacks binding to IgG Fc or VH3.

The invention refers to a monoclonal antibody that counteracts Staphylococcus aureus by specifically binding to immunoglobulin-binding proteins (IGBP) of S. aureus.

### BACKGROUND OF THE INVENTION

Staphylococcal Surface Protein A (SpA or Protein A) and Staphylococcal binder of IgG (Sbi) are multifunctional virulence factors that interact with several human proteins, and act mainly as immune evasion molecules (Falugi, 2013; Smith, 2011). It is proposed that by binding to the Fc portion of immunoglobulins, SpA and Sbi protect Staphylococcus aureus from phagocytosis. SpA also binds VH3-type Fab regions of antibodies that leads to B-cell receptor (BCR) cross-linking and thus non-antigen specific B-cell activation and exhaustion. The result is the suppression of the adaptive immune response against staphylococcal infection shown convincingly in murine models (Sasso, 1991; Goodyear and Silverman, 2004&2005).

SpA, a cell wall anchored protein, is one of the most abundant surface proteins of S. aureus. It has five IgG binding domains (IGBP A-E) that share ˜60-90% amino acid sequence identity (FIGS. 3 and 4). These domains are responsible for both the Fc and VH3 Fab binding, although with different amino acids involved at the interaction sites. Sbi has two domains (IGBP Sbi-I and Sbi-II) that bind the Fc region of IgG (IgG-Fc) in a fashion similar to that of SpA and two domains that can bind to the complement protein C3 and promote its futile consumption in the fluid phase (Smith, 2011). The two IGBP domains of Sbi display about 30% amino acid identity between themselves and compared to the five SpA domains (FIGS. 3 and 4).

SpA has several virulence functions via binding to multiple human molecules, such as TNF-alpha Receptor 1 that leads to modulation of pro-inflammatory signaling, or the epidermal growth factor receptor (EGFR) that triggers a chain of events leading to interference with TNF-1 signaling that is of importance for early pneumonia pathogenesis (Gomez, 2004, 2006, 2007). This latter interaction occurs in the IgG binding domains (Gomez, 2006). The interaction of SpA with the von Willebrand Factor is likely associated with adhesion and microembolism phenomena, enhancing bacterial survival in and spread by the bloodstream (Hartleib, 2000). Importantly for this invention, vWF binding is also mediated via the Ig binding domains (O'Seaghdha, 2006).

SpA deletion mutant S. aureus strains display reduced virulence in multiple animal models for staphylococcal diseases suggesting that SpA has an important contribution to disease pathogenesis (Jonsson 1985, Patel 1987, Cheng 2009). The main function of SpA in murine models seems to be its immunosuppressive effects on B cells. It has been elegantly demonstrated that mice produce significantly higher level of antibodies to different S. aureus components (cytotoxins, surface antigens) when a mutant S. aureus strain expressing SpA variants not able to engage with VH3-Fab and Fc parts is used for exposure of mice (Kim, 2010). These mice become protected to subsequent infection by wild type S. aureus strain, but not those exposed to sublethal infection with wild type S. aureus (Kim, 2010).

The B cell super-antigen function of SpA has been recently demonstrated in humans by analysis of the antibody sequence repertoires of B cells in S. aureus infected subjects that showed significant increase in the VH3 expressing B cell pool (from the normal ˜50 up to 80%) suggesting a non-antigen specific B cell proliferation and expansion due to BCR engagement via VH3 Fab binding (Pauli, 2014).

Based on these data, it is expected that antibodies neutralizing the effect of SpA during S. aureus infection is beneficial to the host. Indeed, it was demonstrated that immunization with recombinant SpA protein mutated to loose Fc and VH3 Fab binding activity (SpA_{KKAA}), but not with wild-type SpA protein, induces protection in murine models of S. aureus infection (Kim, 2010; Kim, 2015). Monoclonal antibodies generated by hyridoma technology using mice immunized with the the S_{P}A_{KKAA} but not with wild-type SpA, was shown to lead to protective immunity against infection by wild-type S. aureus strains (Kim, 2012; Kim, 2014, Thammavongsa, 2015). Such results were not achieved with SpA-specific mAbs using wild-type SpA protein as immunogen (Kim, 2012; Kim, 2014).

US2014/0170134A1 describes antibodies to SPA_{KKAA} mutant which bind to five IGBP domains. The mAb designated 3F6 had the highest affinity to the SpA_{KKAA} mutant. The SpA-E domain was found to be less relevant. The E domain was described as the most dissimilar domain and SpA-E domain antibodies were found to be not protective.

Kim et al. (Infection and Immunity 2012, 80(10)3460-3470) describe protein A-specific mouse monoclonal antibodies raised against the SpA_{KKAA} mutant, which bind to the triple-helical bundle fold of the immunoglobulin binding domains.

WO2013/096948A1 discloses anti-microbial mAbs with substitutions in the heavy chain constant regions to attenuate non-immune binding to microbial virulence factors, e.g. selected from immunoglobulin binding domains of S. aureus.

WO01/40306 A1, example 22, discloses IL-8 antibodies and respective sequences, and does not relate to any immunoglobulin-binding protein of S. aureus.

### SUMMARY OF THE INVENTION

It is the objective of the present invention to provide for a protective antibody targeting IGBP of S. aureus. The object is solved by the subject of the present invention.

According to the invention, there is provided a monoclonal antibody that counteracts Staphylococcus aureus by specifically binding to at least one wild-type immunoglobulin-binding proteins (IGBP) of S. aureus comprising a cross-specific CDR binding site recognizing at least three of the IGBP domains selected from the group consisting of Protein A (SpA) domains and immunoglobulin-binding protein (Sbi) domains SpA-A, SpA-B, SpA-C, SpA-D, SpA-E, Sbi-I, and Sbi-II, wherein the antibody has an affinity to bind SpA-E with a K_{D} of less than 5×10⁻⁹M, as determined by a standard optical interferometry method for a F(ab)2 or F(ab′)2 fragment.

The invention specifically provides for a monoclonal antibody that counteracts Staphylococcus aureus, which comprises a CDR binding site specifically binding to the wild-type SpA-E with a K_{D} of less than 5×10⁻⁹M, as determined by a standard optical interferometry method for a F(ab)2 fragment, which CDR binding site is cross-specific further recognizing at least SpA-A and SpA-D.

Specifically, the CDR binding site further recognizes at least one of SpA-B, SpA-C, Sbi-I, and Sbi-II.

According to a specific embodiment, the antibody recognizes at least SpA-E, SpA-A, and SpA-D.

According to a certain aspect, the antibody has a specificity to recognize at least three of the IGBP domains, preferably at least four, five, or six of the IGBP domains, preferably which recognizes at least three of the IGBP domains each with a K_{D} of less than 5×10⁻⁹M, as determined by a standard optical interferometry method for a F(ab)2 fragment, preferably at least four or five of the IGBP each with a K_{D} of less than 5×10⁻⁹M.

Specifically, the antibody recognizes at least the SpA-E, SpA-A and SpA-D, each with a K_{D} of less than 5×10⁻⁹M, as determined by a standard optical interferometry method for a F(ab)2 fragment.

According to another specific embodiment, the antibody recognizes at least SpA-E, SpA-A, SpA-B, and SpA-D, SpA-C, Sbi-I, and Sbi-II.

According to another specific embodiment, the antibody recognizes at least SpA-E, SpA-A, SpA-B, SpA-D, and Sbi-I.

According to another specific embodiment, the antibody recognizes at least SpA-E, SpA-A, SpA-B, SpA-C, SpA-D, and Sbi-I.

According to another specific embodiment, the antibody recognizes at least SpA-E, SpA-A, SpA-B, SpA-C, SpA-D, Sbi-I, and Sbi-II. Specifically, the antibody recognizes at least three of the IGBP domains each with a K_{D} of less than 5×10⁻⁹M, preferably at least four or five of the IGBP each with a K_{D} of less than 5×10⁻⁹M.

Specifically, the antibody recognizes both, SpA and Sbi, preferably each with a K_{D} of less than 5×10⁻⁹M.

Specifically, the antibody recognizes the wild-type SpA, which is characterized by an amino acid sequence as identified herein, specifically, which comprises the consensus amino acid sequences identified by SEQ ID 145 and 146, with substantially the same affinity or with higher affinity as compared to the mutant SpA_{KKAA}, which comprises a mutation to reduce the SpA binding to IgG Fc and/or VH3. Specifically, the mutant SpA comprises at least one domain comprising the sequence identified by SEQ ID 147 and optionally further comprising the sequence identified by SEQ ID 148. Specifically, the mutant SpA is a SpA_{KKAA} which is a SpA-domain comprising the sequence identified by SEQ ID 147 and further comprising the sequence identified by SEQ ID 148. Specifically, the binding affinity is determined by comparing the affinity to bind the wild-type SpA-D comprising the amino acid sequence SEQ ID 138 and the mutant _{SpA-DKKAA} comprising the amino acid sequence SEQ ID 143.

According to one embodiment, the antibody is capable of binding the wild-type and the mutant SPA_{KKAA} or SpA_{KK} with at least substantially the same affinity, e.g. wherein the dissociation constant ratio K_{D} (SPA_{KKAA})/K_{D} (SpA) or the ratio K_{D} (SpA_{KK})/K_{D} (SpA), e.g. as determined by the binding to the SpA-D_{KKAA} or SpA-D_{KK} compared to the SpA-D (wild-type) is at least 0.5, or at least 0.75, or about 1 or at least 1.

According to another embodiment, the antibody is capable of binding the wild-type and the mutant SpA_{KKAA} or SpA_{KK} with substantially higher affinity, e.g. wherein the dissociation constant ratio K_{D} (SPA_{KKAA})/K_{D} (SpA) or the ratio K_{D} (SpA_{KK})/K_{D} (SpA), e.g. as determined by the binding to the SpA-D_{KKAA} or SPA-D_{KK} compared to the SpA-D (wild-type) is at least 2, or at least 3, or at least 4, or at least 5.

The target antigen of the anti-IGBP antibody is understood as any of the S. aureus IgG binding domains of SpA or Sbi, or a specific selection of the domains as further described herein. Specifically, at least SpA-E and at least one or two further of the IGBP domains selected from the group consisting of SpA-A, SpA-B, SpA-C, SpA-D, Sbi-I, and Sbi-II, are recognized with nanomolar or sub-nanomolar affinity.

Such monoclonal antibodies that inhibit the Fc-binding activity of SpA and Sbi are expected to enhance binding of serum IgGs to the surface antigens of S. aureus via their complementary determining regions (CDRs) rather than being inactivated by non-immune binding through their Fc region.

Specifically, the antibody competes with SpA- and optionally Sbi-binding to IgG-Fc. Thus, the antibody specifically is interfering with the IGBP binding to the IgG-Fc of IgG, i.e. inhibiting the binding or reducing the binding of the IGBP to the natural ligand IgG-Fc, thereby reducing the non-immune interaction of the IGBP with serum immunoglobulins. Specifically, the antibody has a higher affinity to bind the target antigen (i.e. any of the SpA or Sbi, or respective domains) than the non-immune binding of IgG-Fc by the SpA or Sbi, e.g. as determined by comparing affinities of the individual IGBP domains. The non-immune IgG-Fc binding by SpA or Sbi is specifically determined by the IgG-Fc binding region which comprises the following consensus sequence:

SEQ ID 145:
QQXAFYXXL

Wherein

X at position 3 is any of N, S, or K

X at position 7 is any of E, Q, or N, and

X at position 8 is any of I or V

Specifically, the antibody is counteracting or neutralizing Staphylococcus aureus by enhanced opsonophagocytosis and killing by phagocytic cells. A specific test for determining this activity of the antibody is to enumerate live bacteria after incubation with antibody (opsonization) followed by co-incubation with professional phagocytes such as human neutrophil granulocytes. Phagocytes take then up the opsonized pathogen via Fc-receptors which typically results in internalization and intracellular killing of the bacterium (see example 6).

Specifically, the antibody is cross-reactive between different SpA and Sbi variants. Specific antibodies can bind to IGBP variants of at least two strains selected from the group consisting of the strains USA300 TCH1516, MSSA476, JH1, Newman strain, JH9, MW2, Mu3, MRSA252, N315, Mu50, NCTC8325, COL, and USA300_FPR3757. Specific antibodies can bind the IGBP variants of at least one MSSA strain and at least one MRSA strain. Specific antibodies can bind the IGBP variants of at least two strains which are MRSA strains.

According to a specific aspect, the antibody exhibits neutralization potency against the virulence functions of SpA and Sbi, such as Fc and VH3 binding, binding to von Willebrand factor in an in vitro assay with an IC50 of less than 100:1 mAb:protein ratio (mol/mol), preferably less than 50:1, preferably less than 25:1, preferably less than 10:1, more preferably less than 1:1.

According to a further specific aspect, the antibody binds to S. aureus in animals, including both, human and non-human animals, and inhibits S. aureus pathogenesis in vivo, preferably any models of pneumonia, bacteremia, sepsis, abscess, skin infection, peritonitis, catheter and prothetic devices related infection and osteomyelitis.

Specifically, the antibody is a full-length monoclonal antibody, an antibody fragment thereof comprising at least one antibody domain incorporating the binding site, or a fusion protein comprising at least one antibody domain incorporating the binding site, specifically wherein the antibody is a non-naturally occurring antibody which comprises a randomized or artificial amino acid sequence. Preferably, the antibody is selected from the group consisting of murine, chimeric, humanized or human antibodies, heavy-chain antibodies, Fab, F(ab′)2, Fd, scFv and single-domain antibodies like VH, VHH or VL, preferably a human IgG1 antibody, or a human antibody comprising a IgG-Fc mutation, e.g. to reduce binding of IGBP or SpA to the Fc, such as human IgG3.

Specifically, the antibody comprises variable regions and/or variable domains, which comprise CDR and a structure to bind a target antigen through the CDR antigen-binding site, and further comprises constant regions and/or constant domains, e.g. including a (human) framework, e.g. of any of full-length antibodies, heavy-chain antibodies, Fab, F(ab′)2, Fd, scFv and single-domain antibodies like VH, VHH or VL.

Specifically, the antibody comprises at least an antibody heavy chain variable region (VH), which is characterized by any of the CDR1 to CDR3 sequences as listed in Table 1, and optionally an antibody light chain region (VL), which is characterized by any of the CDR4 to CDR6 sequences as listed in Table 1, which CDR sequences are designated according to the numbering system of Kabat, or functionally active CDR variants of any of the foregoing.

Specifically, the antibody comprises at least an antibody heavy chain variable region (VH) and an antibody light chain region (VL), which antibody is characterized by any of the CDR1 to CDR3 sequences as listed in Table 1, and optionally further characterized by any of the CDR4 to CDR6 sequences as listed in Table 1, which CDR sequences are designated according to the numbering system of Kabat, or functionally active CDR variants of any of the foregoing.

While the antibody may be provided as an antibody comprising a binding site determined by CDR sequences of the VH sequence only, e.g. a VH antibody or a heavy chain antibody, according to a specific aspect, the binding site may be further determined by CDR sequences of the antibody light chain variable region (VL), preferably which comprises any of the CDR4 to CDR6 sequences as listed in Table 1, or functionally active CDR variants thereof.

Specifically, the antibody comprises a VH and a VL region and the CDR binding site is characterized by the CDR1, CDR2, and CDR3 of VH and the CDR4, CDR5, and CDR6 of VL.

Specifically, the antibody comprises VH, which is characterized by the CDR1 to CDR3 sequences of any of the antibodies listed in Table 1, and a VL characterized by the CDR4 to CDR6 sequences of any of the antibodies as listed in Table 1, which CDR sequences are designated according to the numbering system of Kabat, or functionally active CDR variants of any of the foregoing.

Specifically the antibody is characterized by the six CDR sequences of any of the antibodies listed in Table 1, or a functionally active CDR variant of any of the antibodies listed in Table 1.

Specifically, the antibody is any of the exemplary antibodies characterized by the heavy chain sequences and optionally further characterized by the light chain sequence listed in FIG. 1b. Specifically, the antibody comprises any of the HC sequences SEQ ID 152-162, and optionally the LC sequence SEQ ID 163.

Specifically, the antibody comprises six CDR sequences, characterized as follows:

VH CDR1:
(SEQ ID 164)
YTFXXXYXH,

wherein

X at position 4=any of T, R, Q, P, D, E, G, S, A, M;

X at position 5=any of S, R, A, E, H, L, G;

X at position 6=any of Y, L, R, H;

X at position 8=any of I, M;

VH CDR2:
(SEQ ID 165)
XINPXXXXTXYAQKFQG,

wherein

X at position 1=any of I, W;

X at position 5=any of S, H, N, P, R, M, G;

X at position 6=any of G, V, N, S, L, Y, I, V, F;

X at position 7=any of G, D;

X at position 8=any of S, H, N, R, G;

X at position 10=any of S, H, N;

VH CDR3 is selected from the group consisting of: SEQ ID 3, SEQ ID 6, SEQ ID 9, SEQ ID 24, SEQ ID 36, SEQ ID 51, and SEQ ID 151;

VL CDR1 (CDR4):
(SEQ ID 166)
XASQXXSXXLX,

wherein

X at position 1=any of R, Q;

X at position 5=any of S, D;

X at position 6=any of V, I;

X at position 8=any of 5, N;

X at position 9=any of S, Y, N;

X at position 11=any of A, N;

VL CDR2 (CDR5):
(SEQ ID 167)
XASXXXX,

wherein

X at position 1=any of G, A, D;

X at position 4=any of T, S, N;

X at position 5=any of R, L;

X at position 6=any of A, Q, E;

X at position 7=any of T, S;

and

VL CDR3 (CDR6) selected from the group consisting of: SEQ ID 63, SEQ ID 66, SEQ ID 69, SEQ ID 84, SEQ ID 87, SEQ ID 96, and SEQ ID 111.

Specifically, the antibody
a) comprises a VH domain, which is characterized by any of the CDR1 to CDR3 sequence combinations as listed in Table 1, and a VL domain, which is characterized by any of the CDR4 to CDR6 sequence combinations as listed in Table 1;
b) comprises the set of CDR sequences (CDR1-CDR6) of any of the antibodies as listed in Table 1;
c) is any of the antibodies as listed in Table 1; or
d) is a functionally active variant of a parent antibody that is characterized by the sequences of a)-c),

preferably wherein
i. the functionally active variant comprises at least one functionally active CDR variant of any of the CDR1-CDR6 of the parent antibody; and/or
ii. the functionally active variant comprises at least one point mutation in the framework region of any of the VH and VL sequences;

and further wherein
iii. the functionally active variant has a specificity to bind the same epitope as the parent antibody; and/or
iv. the functionally active variant is a human, humanized, chimeric or murine and/or affinity matured variant of the parent antibody.

Specifically, the antibody comprises a functionally active CDR variant of any of the CDR sequences as listed in Table 1, wherein the functionally active CDR variant comprises at least one of

a) 1, 2, or 3 point mutations in the parent CDR sequence; and/or

b) 1 or 2 point mutations in any of the four C-terminal or four N-terminal, or four centric amino acid positions of the parent CDR sequence; and/or

c) at least 60% sequence identity with the parent CDR sequence;

preferably wherein the functionally active CDR variant comprises 1 or 2 point mutations in any CDR sequence.

Specifically, the antibody is selected from the group consisting of group members i) to vi), wherein

i)

A) the antibody comprises
a) a CDR1 consisting of the amino acid sequence of SEQ ID 13; and
b) a CDR2 consisting of the amino acid sequence of SEQ ID 14; and
c) a CDR3 consisting of the amino acid sequence of SEQ ID 15;

and optionally further comprises
d) a CDR4 consisting of the amino acid sequence of SEQ ID 73; and
e) a CDRS consisting of the amino acid sequence of SEQ ID 74; and
f) a CDR6 consisting of the amino acid sequence of SEQ ID 75;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 13;
b) the parent CDR2 consists of the amino acid sequence SEQ ID 14;
c) the parent CDR3 consists of the amino acid sequence SEQ ID 15;
d) the parent CDR4 consists of the amino acid sequence SEQ ID 73;
e) the parent CDRS consists of the amino acid sequence SEQ ID 74;
f) the parent CDR6 consists of the amino acid sequence SEQ ID 75;

ii)

A) the antibody comprises
a) a CDR1 consisting of the amino acid sequence of SEQ ID 31; and
b) a CDR2 consisting of the amino acid sequence of SEQ ID 32; and
c) a CDR3 consisting of the amino acid sequence of SEQ ID 33;

and optionally further comprises
d) a CDR4 consisting of the amino acid sequence of SEQ ID 91; and
e) a CDRS consisting of the amino acid sequence of SEQ ID 92; and
f) a CDR6 consisting of the amino acid sequence of SEQ ID 93;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 31;
b) the parent CDR2 consists of the amino acid sequence SEQ ID 32;
c) the parent CDR3 consists of the amino acid sequence SEQ ID 33;
d) the parent CDR4 consists of the amino acid sequence SEQ ID 91;
e) the parent CDR5 consists of the amino acid sequence SEQ ID 92;
f) the parent CDR6 consists of the amino acid sequence SEQ ID 93;

iii)

A) the antibody comprises
a) a CDR1 consisting of the amino acid sequence of SEQ ID 40; and
b) a CDR2 consisting of the amino acid sequence of SEQ ID 41; and
c) a CDR3 consisting of the amino acid sequence of SEQ ID 42;

and optionally further comprises
d) a CDR4 consisting of the amino acid sequence of SEQ ID 100; and
e) a CDR5 consisting of the amino acid sequence of SEQ ID 101; and
f) a CDR6 consisting of the amino acid sequence of SEQ ID 102;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 40;
b) the parent CDR2 consists of the amino acid sequence SEQ ID 41;
c) the parent CDR3 consists of the amino acid sequence SEQ ID 42;
d) the parent CDR4 consists of the amino acid sequence SEQ ID 100;
e) the parent CDR5 consists of the amino acid sequence SEQ ID 101;
f) the parent CDR6 consists of the amino acid sequence SEQ ID 102;

iv)

A) the antibody comprises
a) a CDR1 consisting of the amino acid sequence of SEQ ID 43; and
b) a CDR2 consisting of the amino acid sequence of SEQ ID 44; and
c) a CDR3 consisting of the amino acid sequence of SEQ ID 45;

and optionally further comprises
d) a CDR4 consisting of the amino acid sequence of SEQ ID 103; and
e) a CDR5 consisting of the amino acid sequence of SEQ ID 104; and
f) a CDR6 consisting of the amino acid sequence of SEQ ID 105;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 43;
b) the parent CDR2 consists of the amino acid sequence SEQ ID 44;
c) the parent CDR3 consists of the amino acid sequence SEQ ID 45;
d) the parent CDR4 consists of the amino acid sequence SEQ ID 103;
e) the parent CDR5 consists of the amino acid sequence SEQ ID 104;
f) the parent CDR6 consists of the amino acid sequence SEQ ID 105;

v)

A) the antibody comprises
a) a CDR1 consisting of the amino acid sequence of SEQ ID 46; and
b) a CDR2 consisting of the amino acid sequence of SEQ ID 47; and
c) a CDR3 consisting of the amino acid sequence of SEQ ID 48;

and optionally further comprises
d) a CDR4 consisting of the amino acid sequence of SEQ ID 106; and
e) a CDR5 consisting of the amino acid sequence of SEQ ID 107; and
f) a CDR6 consisting of the amino acid sequence of SEQ ID 108;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 46;
b) the parent CDR2 consists of the amino acid sequence SEQ ID 47;
c) the parent CDR3 consists of the amino acid sequence SEQ ID 48;
d) the parent CDR4 consists of the amino acid sequence SEQ ID 106;
e) the parent CDR5 consists of the amino acid sequence SEQ ID 107;
f) the parent CDR6 consists of the amino acid sequence SEQ ID 108;

and

vi)

A) the antibody comprises
a) a CDR1 consisting of the amino acid sequence of SEQ ID 58; and
b) a CDR2 consisting of the amino acid sequence of SEQ ID 59; and
c) a CDR3 consisting of the amino acid sequence of SEQ ID 60;

and optionally further comprises
d) a CDR4 consisting of the amino acid sequence of SEQ ID 118; and
e) a CDR5 consisting of the amino acid sequence of SEQ ID 119; and
f) a CDR6 consisting of the amino acid sequence of SEQ ID 120;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 58;
b) the parent CDR2 consists of the amino acid sequence SEQ ID 59;
c) the parent CDR3 consists of the amino acid sequence SEQ ID 60;
d) the parent CDR4 consists of the amino acid sequence SEQ ID 118;
e) the parent CDR5 consists of the amino acid sequence SEQ ID 119;
f) the parent CDR6 consists of the amino acid sequence SEQ ID 120.

The invention further provides for the medical use of the antibody as described herein, and the respective method of treatment or method of manufacturing a preparation for medical use.

According to the invention the antibody is further provided for use in treating a subject at risk of or suffering from S. aureus infection or colonization comprising administering to the subject an effective amount of the antibody to limit the infection in the subject or to ameliorate a disease condition resulting from said infection or to inhibit S. aureus disease pathogenesis, such as pneumonia, sepsis, bacteremia, wound infection, abscesses, surgical site infection, endothalmitis, furunculosis, carbunculosis, endocarditis, peritonitis, osteomyelitis or joint infection.

According to the invention, there is further provided a pharmaceutical preparation comprising the antibody as described herein, preferably comprising a parenteral or mucosal formulation, optionally containing a pharmaceutically acceptable carrier or excipient. The preparation of an antibody as described herein specifically comprises the antibody in the isolated form, optionally in a pharmaceutically acceptable formulation.

Specifically, the pharmaceutical preparation is provided for protecting against pathogenic S. aureus or against S. aureus infections.

Specifically, the pharmaceutical preparation may comprise the antibody as a sole active substance, or in combination with other active substance(s), or a cocktail of active substance(s), such as a combination or cocktail to administer one or more further antibodies, e.g. further targeting S. aureus, e.g. an (SPK antibody or an antibody targeting at least one toxin. Specifically, a cocktail of antibodies comprises one or more antibodies as described herein in a mixture, and optionally further active substances.

According to the invention an antibody is further provided for diagnostic use to detect any S. aureus infections, including high toxin producing MRSA infections, such as necrotizing pneumonia, and toxin production in furunculosis and carbunculosis.

Specifically, the antibody is provided for diagnostic use, wherein a systemic infection with S. aureus in a subject is determined ex vivo by contacting a sample of body fluid of said subject with the antibody, wherein a specific immune reaction of the antibody determines the infection.

Therefore, the invention further refers to the respective method of diagnosing an S. aureus infection in a subject, in particular wherein a systemic infection with S. aureus in a subject is determined.

According to the invention, there is further provided a diagnostic preparation of an antibody as described herein, optionally containing the antibody with a label and/or a further diagnostic reagent with a label.

According to the invention, there is further provided an isolated nucleic acid encoding an antibody as described herein.

According to the invention, there is further provided a recombinant expression cassette or a plasmid comprising a coding sequence to express a light chain and/or heavy chain of an antibody as described herein.

According to the invention, there is further provided a host cell comprising the expression cassette or the plasmid as described herein.

Specifically preferred is a host cell and a production method employing such host cell, which host cell comprises
the plasmid or expression cassette of the invention, which incorporates a coding sequence to express the antibody light chain; and
the plasmid or expression cassette of the invention, which incorporates a coding sequence to express the antibody heavy chain.

According to the invention, there is further provided a method of producing an antibody as described herein, wherein a host cell is cultivated or maintained under conditions to produce said antibody.

According to the invention, there is further provided a method of producing a functionally active variant antibody of a parent antibody which parent antibody is any of the antibodies characterized by the CDR sequences as listed in Table 1, which method comprises engineering at least one point mutation in any of the framework regions (FR) or constant domains, or any of the CDR sequences of the parent antibody to obtain a variant antibody, and determining the functional activity of the variant antibody by its affinity to bind SpA and/or Sbi with a K_{D} of less than 10⁻⁸M, preferably less than 5×10⁻⁹M, wherein upon determining the functional activity, the functionally active variant antibody is selected for production by a recombinant production method.

The invention further provides for a method of producing an antibody as described herein, comprising

(a) immunizing a non-human animal with the three-dimensional structure of the wild-type IGBP of Staphylococcus aureus;

(b) forming immortalized cell lines from the isolated B-cells;

(c) screening the cell lines to identify a cell line producing a monoclonal antibody that binds to the IGBP and optionally further IGBP which are different SpA or Sbi domains or variants; and

(d) producing the monoclonal antibody, or a humanized or human form of the antibody, or a derivative thereof with the same epitope binding specificity as the monoclonal antibody.

According to a further aspect, the invention provides for a method of producing an antibody as described herein, comprising

(a) immunizing a non-human animal with the wild-type IGBP of Staphylococcus aureus and isolating B-cells producing antibodies;

(b) forming immortalized cell lines from the isolated B-cells;

(c) screening the cell lines to identify a cell line producing a monoclonal antibody that binds to the IGBP and optionally further IGBP which are different SpA or Sbi domains or variants; and

(d) producing the monoclonal antibody, or a humanized or human form of the antibody, or a derivative thereof with the same epitope binding specificity as the monoclonal antibody.

A series of antibodies is herein described as exemplary antibodies as listed in FIG. 1, Table 1, including antibodies of the examples. It is understood that those exemplary antibodies and functionally active variants are included in the subject of the present claims, including, but not limited to, CDR variants, FR variants, murine, chimeric, humanized or human variants, or any antibody domain combination other than a combination composed of the VH and VL or the HC (including the VH) and LC (including the VL) as described herein, e.g. an antibody comprising the same CDR1-6 or VHNL combination, yet, with FR sequences of a variety of sources

Herein described are specific functionally active CDR variants of VH or VL sequences, wherein any of the CDR 1-6 sequences is a functionally active CDR variant of a parent CDR, comprising at least one point mutation in the parent CDR and at least 60% sequence identity, or at least 70%, at least 80%, or at least 90% sequence identity.

In certain aspects, the invention also provides for such variant antibodies, comprising the respective binding sequences, such as the variable sequences and/or the CDR sequences, as derived from any of the exemplary antibodies, which are used as parent antibodies, wherein the binding sequences or the CDR comprises a sequence that has at least 60%, preferably at least 70%, or at least 80%, or at least 90%, or at least 95%, or at least 99% identity to the amino acid sequence as derived from the parent antibodies, and wherein the variant is a functionally active variant.

Any of the exemplary antibodies may be used as parent antibodies to produce functionally active antibody variants of such parent antibodies, wherein the functional activity is determined, if the target antigen is bound with high affinity, e.g. with a K_{D} of less than 10⁻⁸M (e.g. in the case of any IGBP domain other than SpA-E), preferably any of less than 5×10⁻⁹M, less than 4×10⁻⁹ M, less than 3×10⁻⁹ M, less than 2×10⁻⁹ M, less than 10⁻⁹ M, less than 5×10⁻¹⁰ M, less than 4×10⁻¹⁰ M, less than 3×10⁻¹⁰ M, less than 2×10⁻¹⁰ M or less than 10⁻¹⁰ M (e.g. in the case of SpA-E), or preferably less than 10⁻⁹M, or preferably less than 5×10⁻¹⁰M, and/or the binding of the variant antibody to the target antigen competes with the binding by the parent antibody, or the variant antibody binds to the same epitope as the parent antibody.

Functionally active variant antibodies may differ in any of the VH or VL sequences, or share the common VH and VL sequences, and comprise modifications in the respective FR. The variant antibody derived from the parent antibody by mutagenesis may be produced by methods well-known in the art.

Exemplary parent antibodies are described in the examples section below and in FIG. 1. Specifically, the preparation as described herein may include a functionally active derivative of a parent antibody as listed in FIG. 1. Variants with one or more modified CDR sequences may be engineered.

CDR combinations may be used as listed in FIG. 1 or different CDR combinations, provided, that the antibody is still functionally active.

Specifically, an antibody as described herein comprises the CDR1-6 of any of the antibodies as listed in FIG. 1. However, according to an alternative embodiment, an antibody may comprise different CDR combinations, e.g. wherein an antibody as listed in FIG. 1 comprises at least one CDR sequence, such as 1, 2, 3, 4, 5, or 6 CDR sequences of one antibody and at least one further CDR sequence of a different antibody of any of the antibodies as listed in FIG. 1. According to a specific example, the antibody comprises 1, 2, 3, 4, 5, or 6 CDR sequences, wherein the CDR sequences are CDR combinations of more than 1 antibody, e.g. 2, 3, 4, 5, or 6 different antibodies. For example, the CDR sequences may be combined to preferably comprise 1, 2, or all 3 of CDR1-3 of any of the antibodies as listed in FIGS. 1, and 1, 2, or all 3 of CDR4-6 of the same or any other antibody listed in FIG. 1.

It is herein specifically understood that the CDRs numbered CDR1, 2, and 3 represent the binding region of the VH domain, and CDR4, 5, and 6 represent the binding region of the VL domain.

Specifically, the functionally active variant differs from a parent antibody, e.g. any of the antibodies as listed in FIG. 1, in at least one point mutation in the amino acid sequence. Specifically, the at least one point mutation is any of an amino acid substitution, deletion and/or insertion of one or more amino acids.

Specifically, the CDR sequence may include at least one point mutation such as to obtain a functionally active CDR variant, e.g. wherein the number of point mutations in each of the CDR amino acid sequences is either 0, 1, 2 or 3.

Specifically, the antibody is derived from such antibodies, employing the respective CDR sequences, or CDR mutants, including functionally active CDR variants, e.g. with 1, 2 or 3 point mutations within one CDR loop, e.g. within a CDR length of 5-18 amino acids, e.g. within a CDR region of 5-15 amino acids or 5-10 amino acids. Alternatively, there may be 1 to 2 point mutations within one CDR loop, e.g. within a CDR length of less than 5 amino acids, to provide for an antibody comprising a functionally active CDR variant. Specific CDR sequences might be short, e.g. the CDR2 or CDR5 sequences. According to a specific embodiment, the functionally active CDR variant comprises 1 or 2 point mutations in any CDR sequence consisting of less than 4 or 5 amino acids.

Specific antibodies are provided as CDR mutated antibodies, e.g. to improve the affinity of an antibody e.g. by affinity maturation, and/or to target the same epitope or epitopes near the epitope that is targeted by a parent antibody (epitope shift).

According to a specific aspect, an antibody as described herein comprises CDR and framework sequences, wherein at least one of the CDR and framework sequences includes human, humanized, chimeric, murine or affinity matured sequences, preferably wherein the framework sequences are of an IgG antibody, e.g. of an IgG1, IgG2, IgG3, or IgG4 subtype, or of an IgA1, IgA2, IgD, IgE, or IgM antibody.

Specific antibodies are provided as framework mutated antibodies, e.g. to improve manufacturability or tolerability of a parent antibody, e.g. to provide an improved (mutated) antibody which has a low immunogenic potential, such as humanized antibodies with mutations in any of the CDR sequences and/or framework sequences as compared to a parent antibody.

### FIGURES

FIG. 1 a. Table 1

Table 1 a: VH CDR sequences;

Table 1 b: VL CDR sequences.

The nomenclature as used herein shall have the following meaning:

VH CDR1=CDR1

VH CDR2=CDR2

VH CDR3=CDR3

VL CDR4=CDR4=VL CDR1

VL CDR5=CDR5=VL CDR2

VL CDR6=CDR6=VL CDR3

Table 1c: Affinity of selected mAbs to bind SpA-E and SpA wild-type versus SpA mutant:

The affinity was measured as follows. Biotinylated SpA-E, SpA-D and SpA-D_{KKAA} were produced as described in Example 1 and F(ab′)₂ fragments were generated from yeast or CHO derived IgGs by pepsin digestion as described in Example 2. Binding of the mAbs to the SpA domains was measured by interferometry using a ForteBio Octet Red instrument [Pall Life Sciences]; The biotinylated antigen (5 μg/ml) was immobilized on streptavidin sensors, to give a sensor loading of ˜2 nm. The association and dissociation of the antibody F(ab′)₂ fragment (50 nm; 100 nM for the yeast derived material with SpA-E), in solution (PBS, pH 7.2 plus 1% BSA), were measured at 30° C. for 10 min (5 min the yeast derived material with SpA-E) for the association and 5 min (3 min the yeast derived material with SpA-E) for the dissociation phase. The dissociation constants (KD values) were calculated based on the kinetic parameters (kon and koff) determined by fitting simultaneously the association and dissociation phases to a 1:1 binding model using Octet Data Analysis Software version 7. The improved binding to WT versus KKAA mutant SpA-D is expressed as KD ratio. NB indicates no binding to the SpA-D mutant.

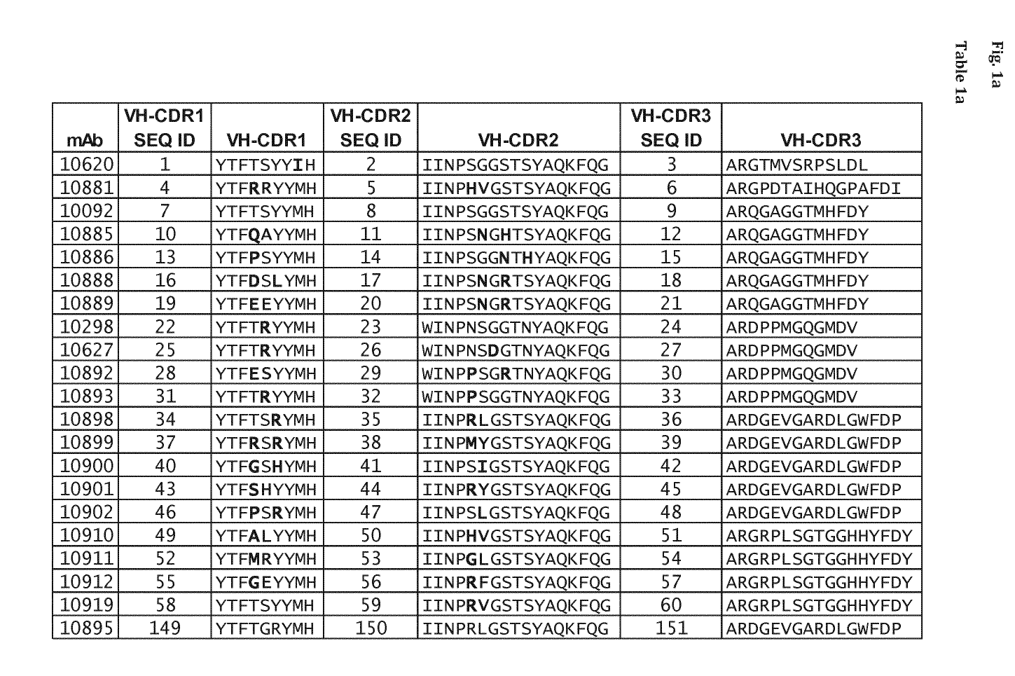

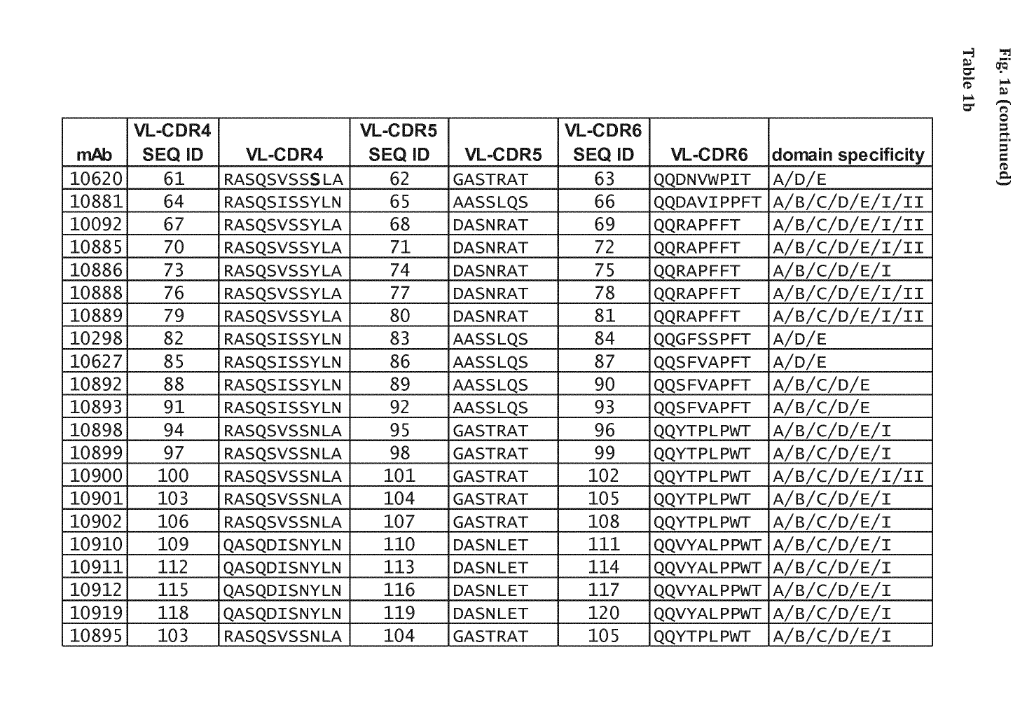

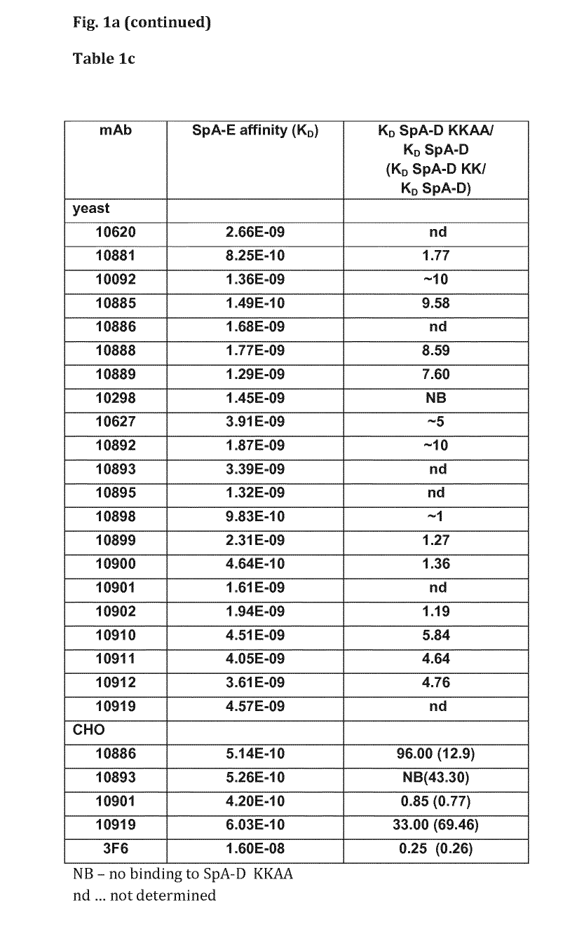

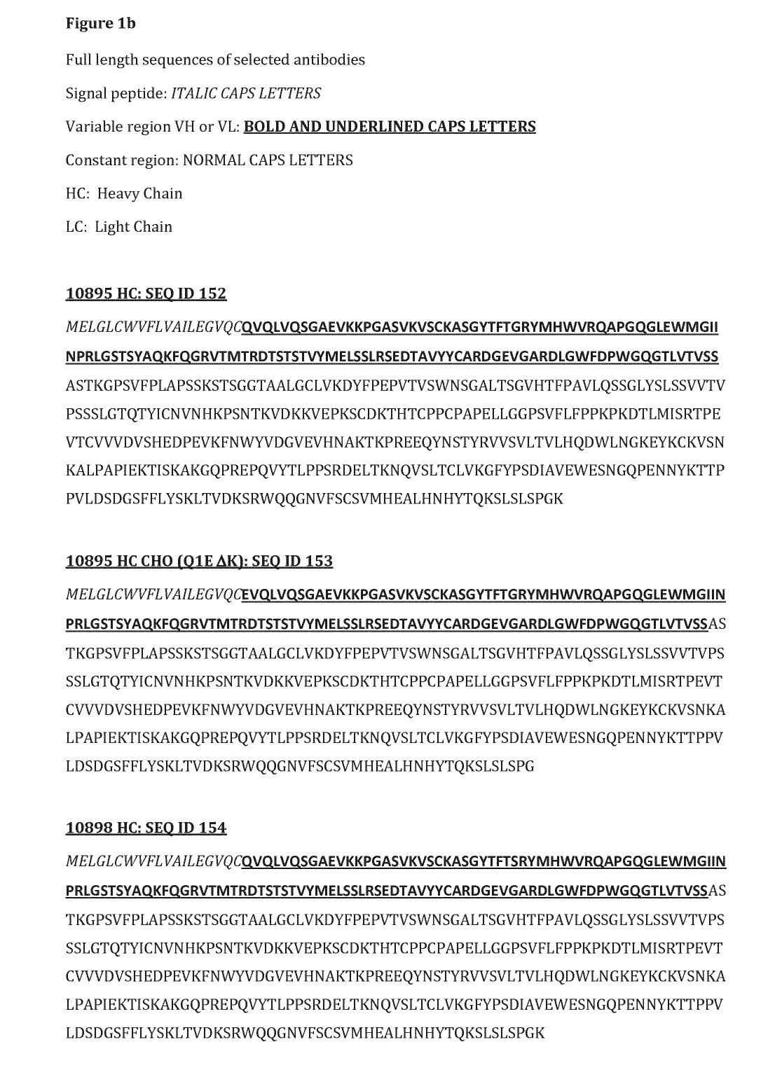

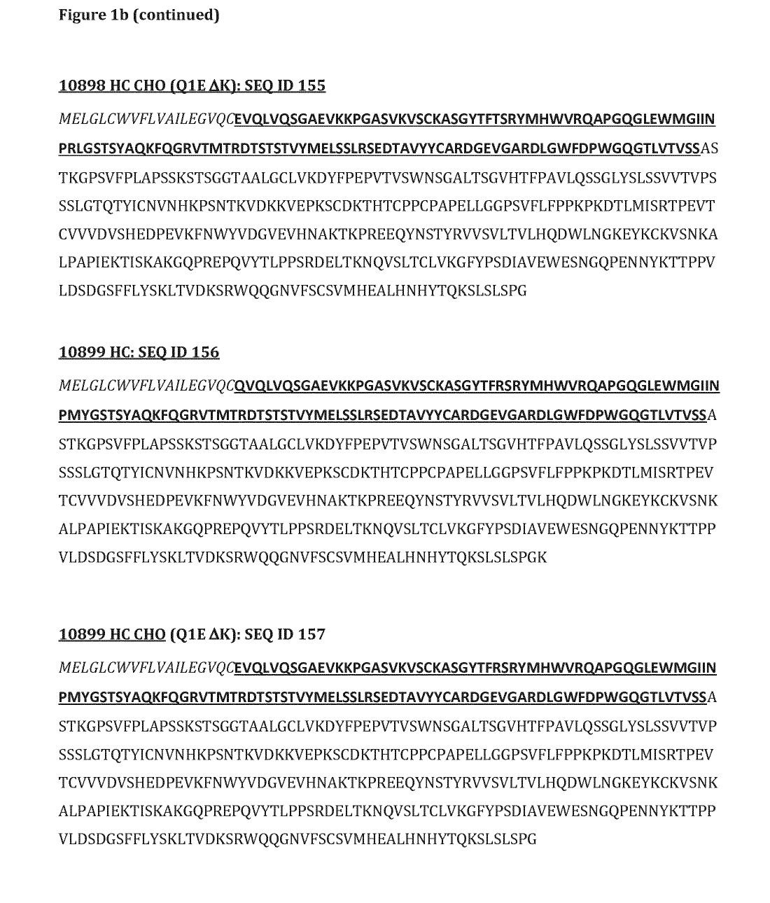

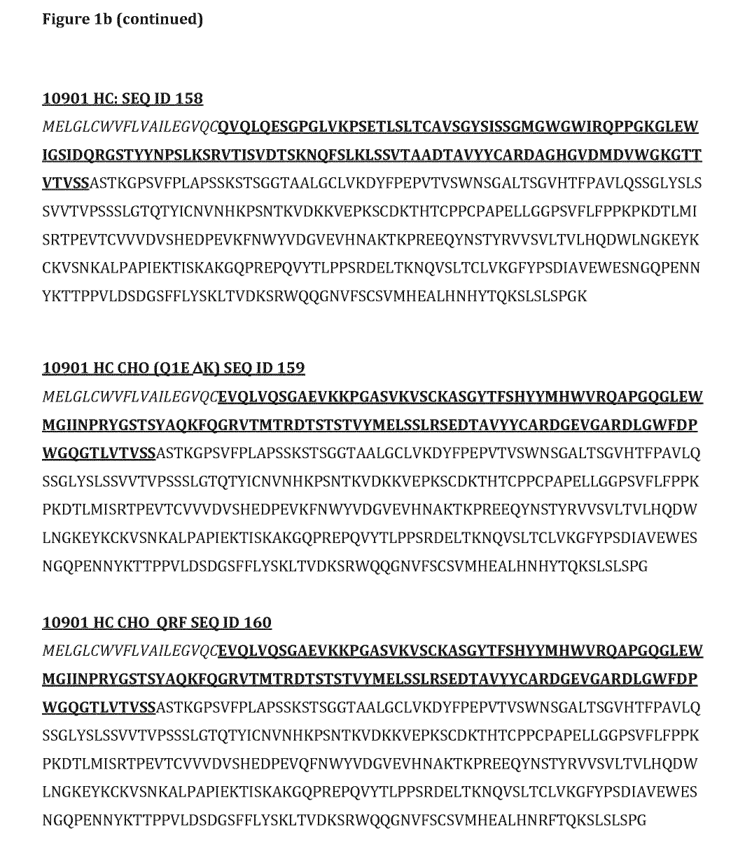

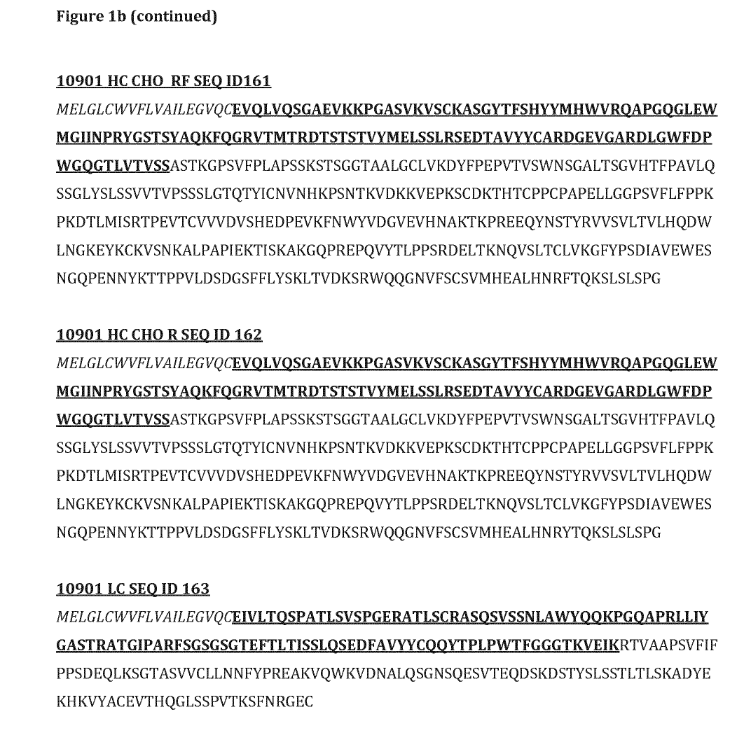

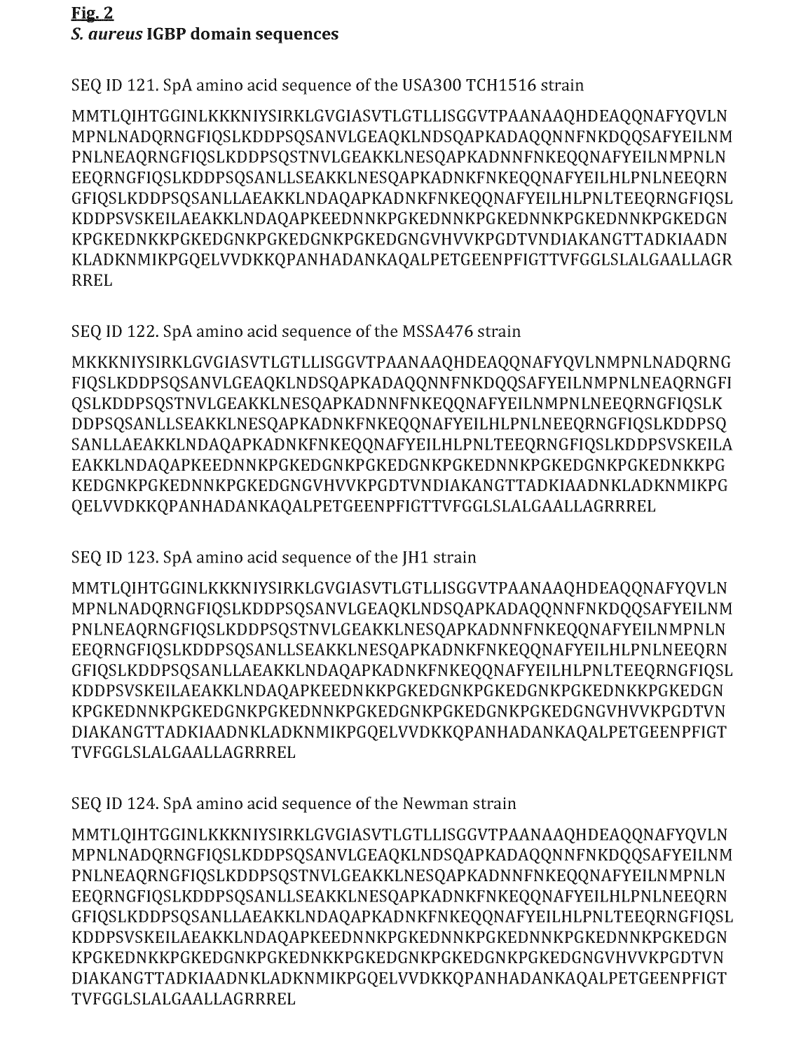

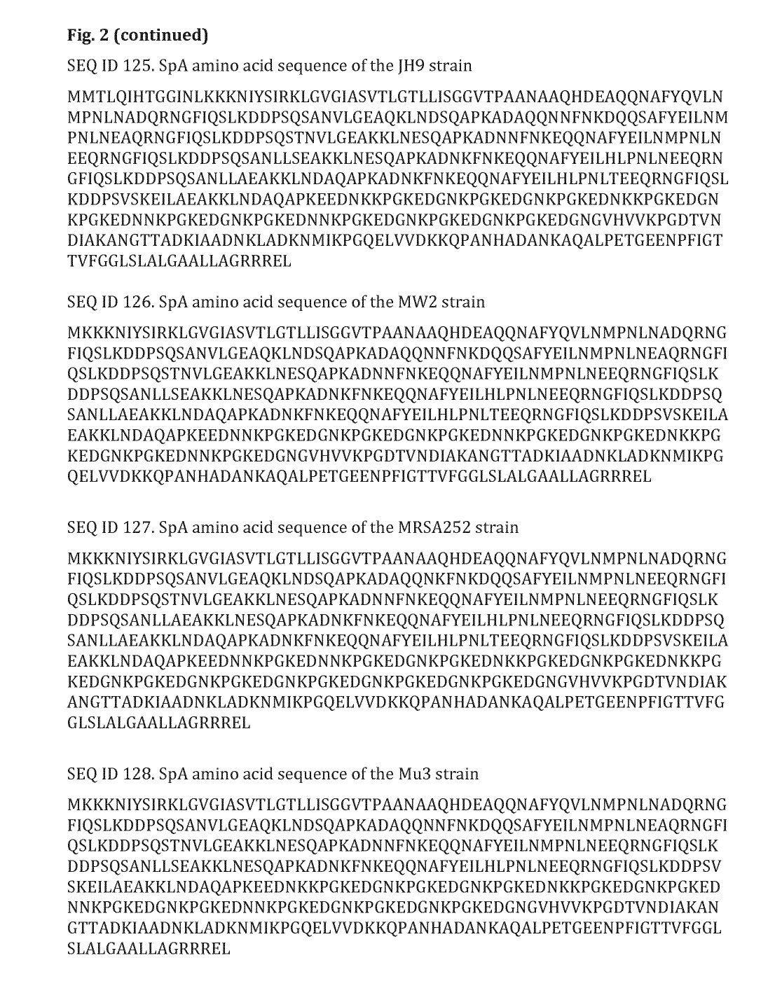

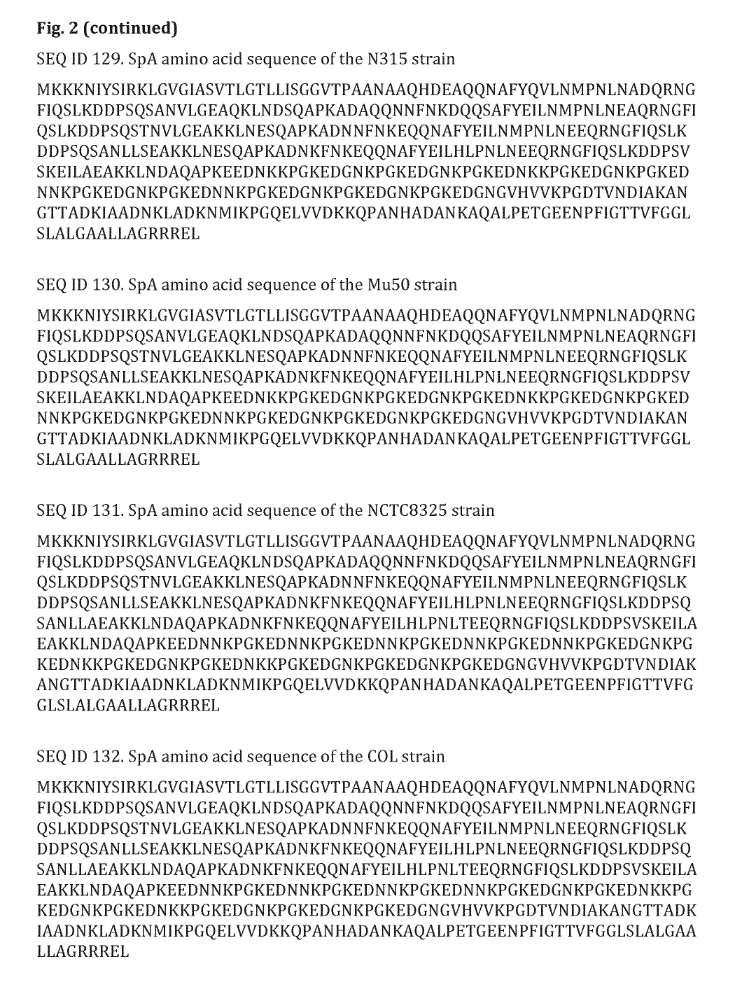

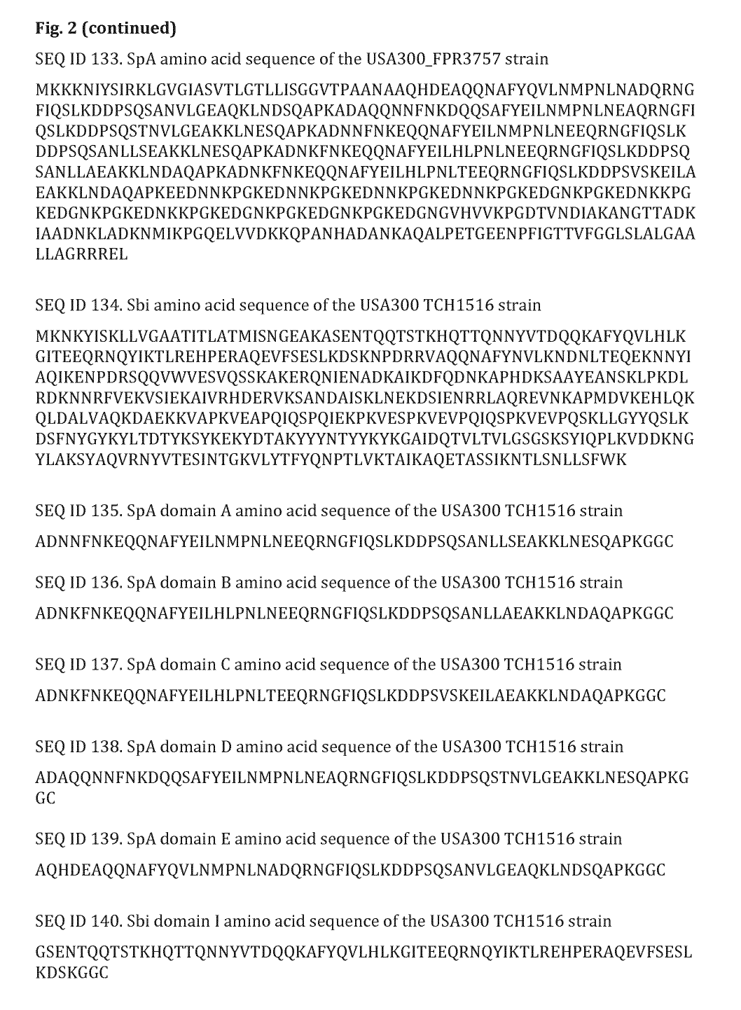

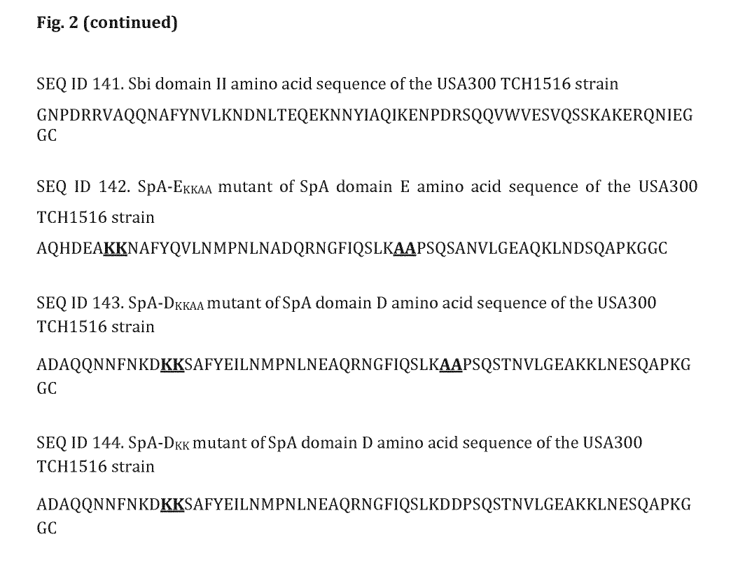

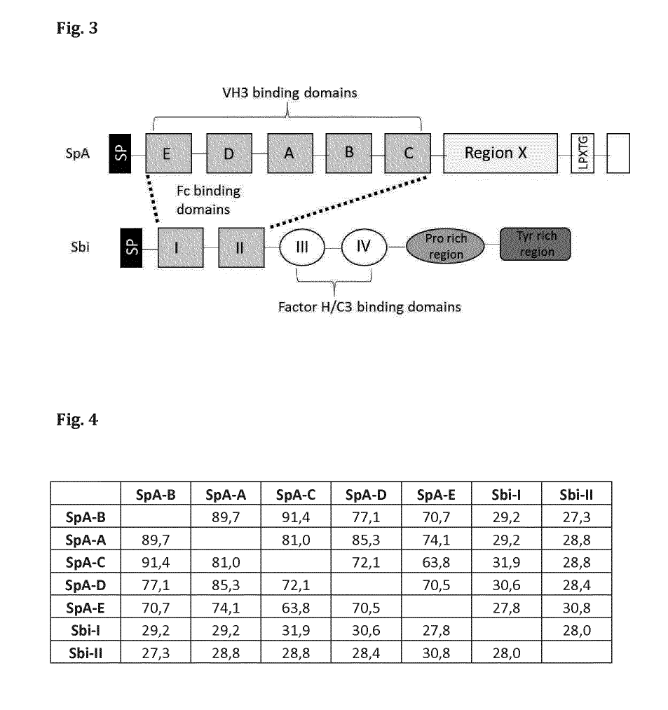

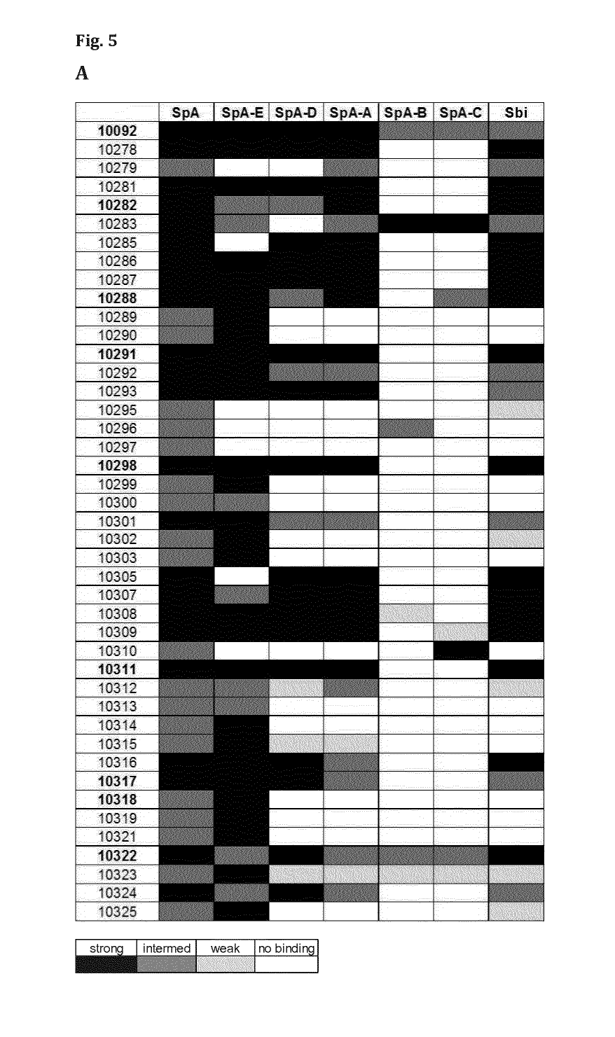

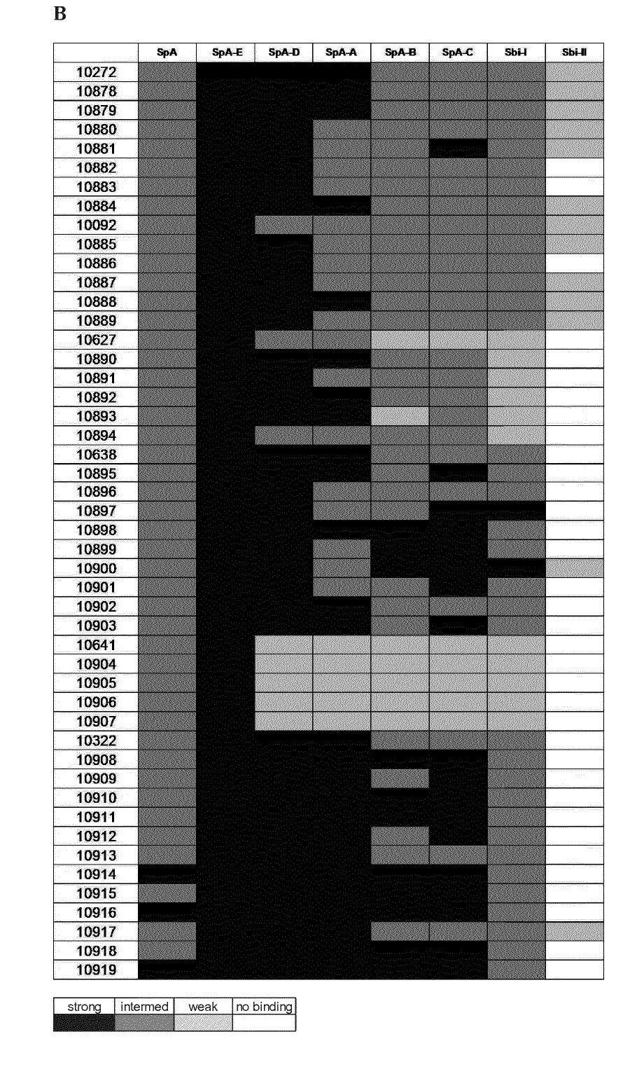

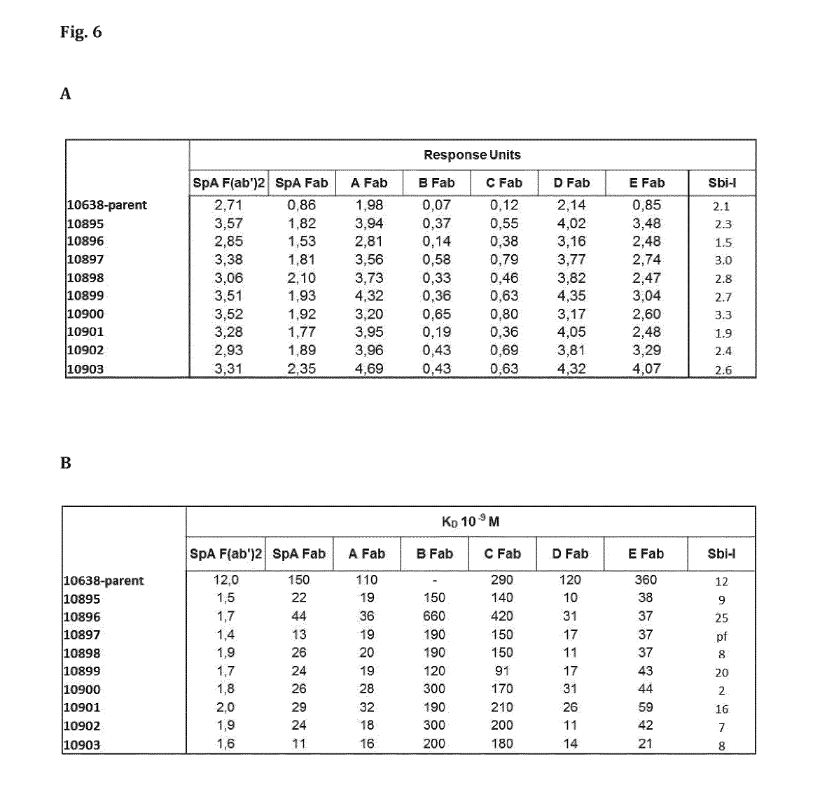

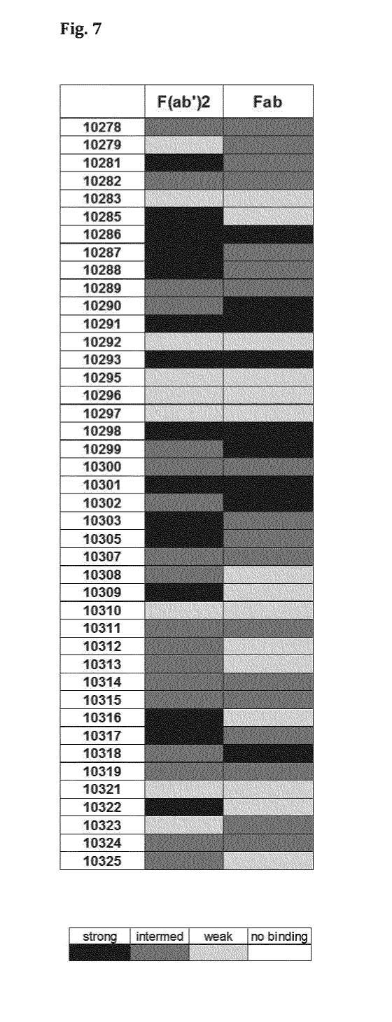

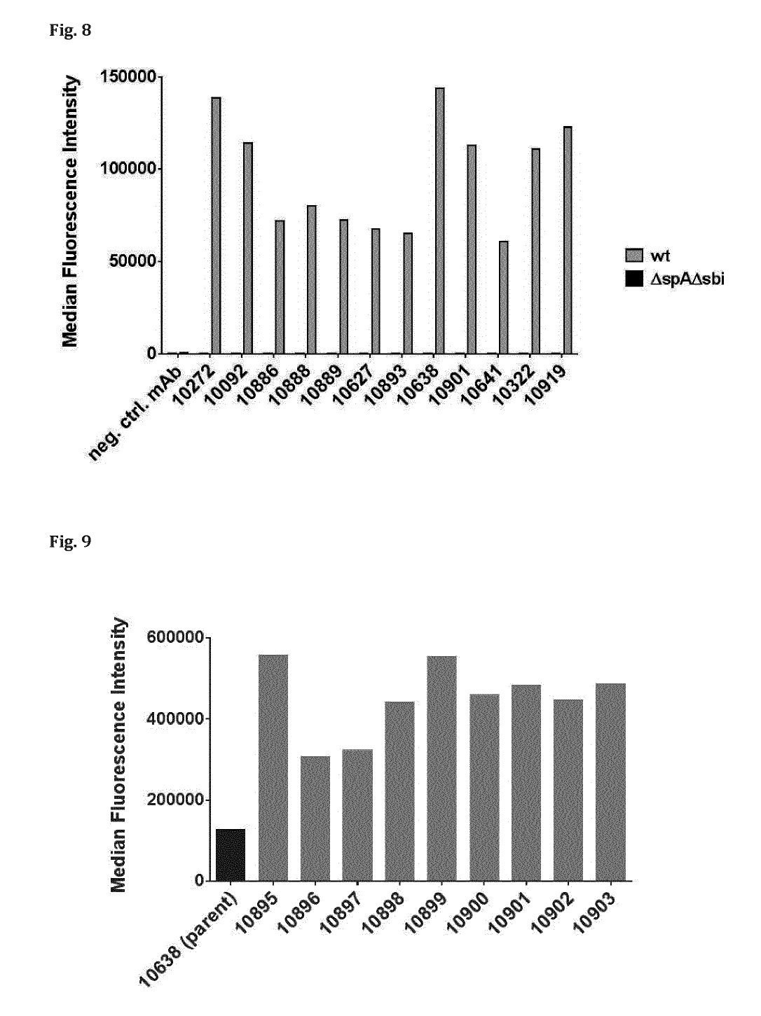

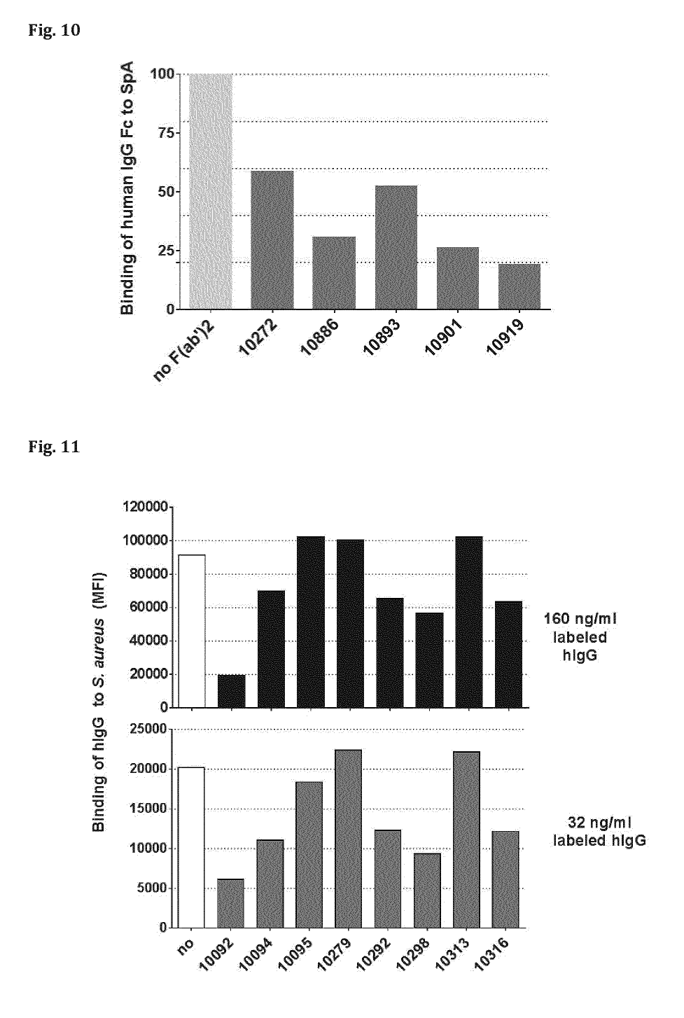

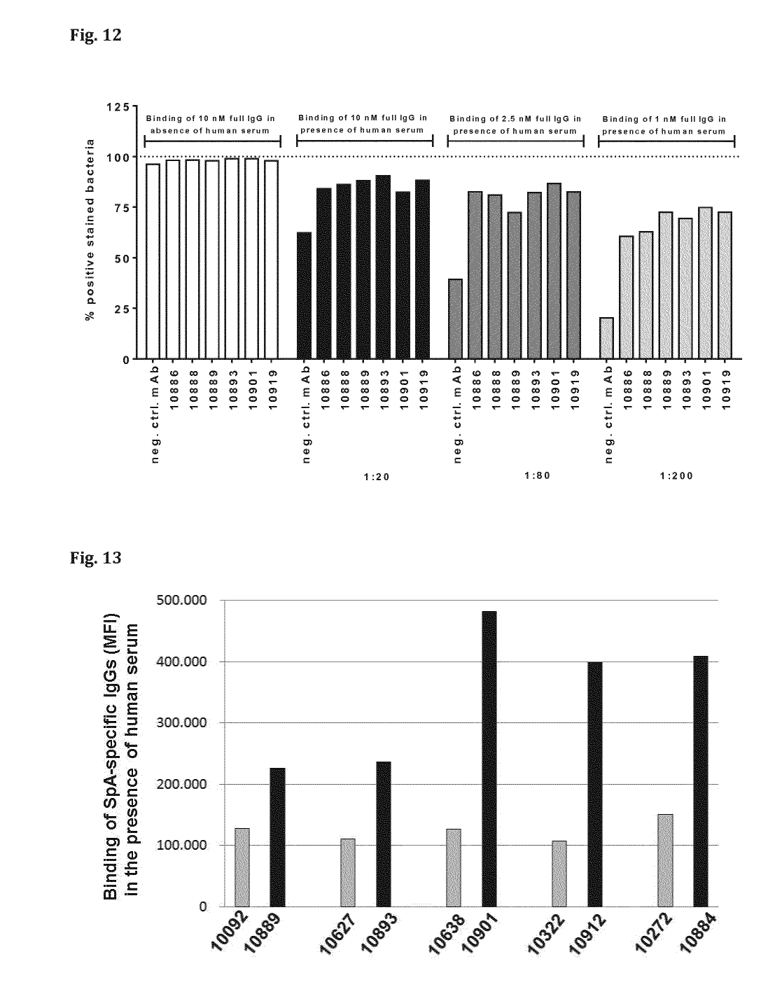

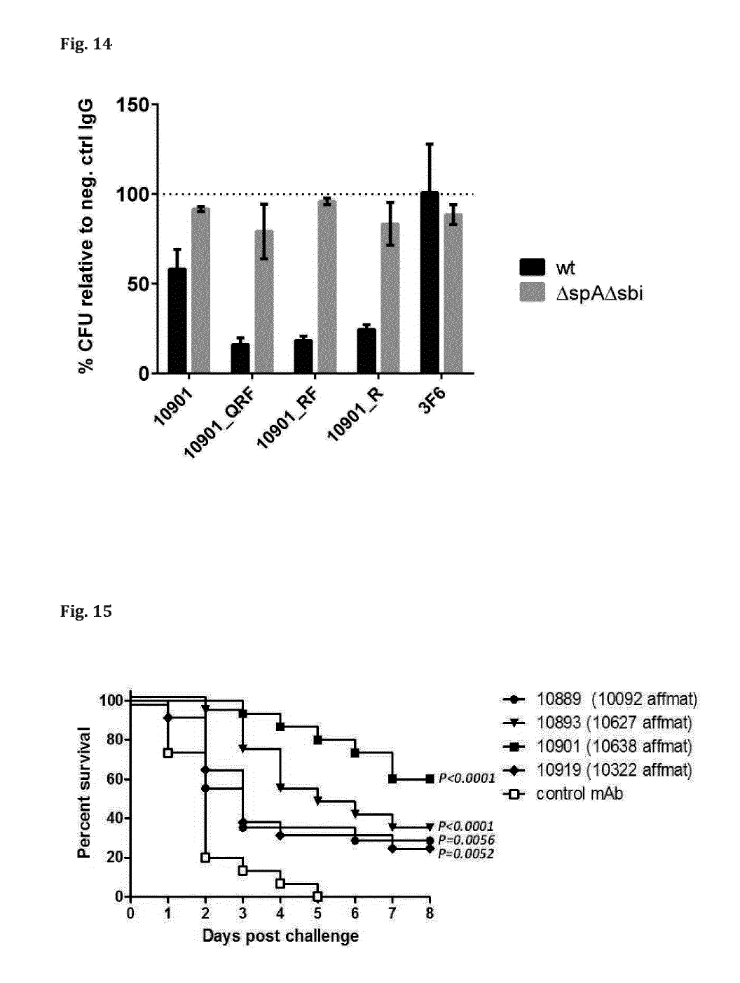

FIG. 1b: Full length sequences of selected mAbs

The heavy chain of selected antibodies is listed (SEQ ID 152-162). All antibodies share the light chain 10901 (SEQ ID 163).

10895 HC: SEQ ID 152

10895 HC CHO (Q1E ΔK): SEQ ID 153

10898 HC: SEQ ID 154

10898 HC CHO (Q1E ΔK): SEQ ID 155

10899 HC: SEQ ID 156

10899 HC CHO (Q1E ΔK): SEQ ID 157

10901 HC: SEQ ID 158

10901 HC CHO (Q1E ΔK) SEQ ID 159

10901 HC CHO QRF SEQ ID 160

10901 HC CHO RF SEQ ID 161

10901 HC CHO R SEQ ID 162

10901 LC SEQ ID 163

FIG. 2. IGBP sequences. The following sequences of IGBP domains may contain a C-terminal GGC tag to facilitate site directed labeling of the antigens. It is understood that the sequences provided herein represent the amino acid sequences of the IGBP domains with or without the GGC tag.

SEQ ID 121. SpA amino acid sequence of the USA300 TCH1516 strain

SEQ ID 122. SpA amino acid sequence of the MSSA476 strain

SEQ ID 123. SpA amino acid sequence of the JH1 strain

SEQ ID 124. SpA amino acid sequence of the Newman strain

SEQ ID 125. SpA amino acid sequence of the JH9 strain

SEQ ID 126. SpA amino acid sequence of the MW2 strain

SEQ ID 127. SpA amino acid sequence of the MRSA252 strain

SEQ ID 128. SpA amino acid sequence of the Mu3 strain

SEQ ID 129. SpA amino acid sequence of the N315 strain

SEQ ID 130. SpA amino acid sequence of the Mu50 strain

SEQ ID 131. SpA amino acid sequence of the NCTC8325 strain

SEQ ID 132. SpA amino acid sequence of the COL strain

SEQ ID 133. SpA amino acid sequence of the USA300_FPR3757 strain

SEQ ID 134. Sbi amino acid sequence of the USA300 TCH1516 strain

SEQ ID 135. SpA domain A amino acid sequence of the USA300 TCH1516 strain

SEQ ID 136. SpA domain B amino acid sequence of the USA300 TCH1516 strain

SEQ ID 137. SpA domain C amino acid sequence of the USA300 TCH1516 strain

SEQ ID 138. SpA domain D amino acid sequence of the USA300 TCH1516 strain

SEQ ID 139. SpA domain E amino acid sequence of the USA300 TCH1516 strain

SEQ ID 140. Sbi domain I amino acid sequence of the USA300 TCH1516 strain

SEQ ID 141. Sbi domain II amino acid sequence of the USA300 TCH1516 strain

SEQ ID 142. SpA-E_{KKAA} mutant of SpA domain E amino acid sequence of the USA300 TCH1516 strain

SEQ ID 143. SpA-D_{KKAA} mutant of SpA domain D amino acid sequence of the USA300 TCH1516 strain

SEQ ID 144. SpA-D_{KK} mutant of SpA domain D amino acid sequence of the USA300 TCH1516 strain

FIG. 3. Domain structure of SpA and Sbi. Schematic drawing of domain organization of SpA and Sbi, described in Example 1.

FIG. 4. Sequence homology among IgG binding domains of SpA and Sbi: percent sequence identity of the five SpA and two Sbi domains from the USA300 TCH1516 strain as described in Example 1.

FIG. 5. Binding response of anti-SpA (A) na\dot{i}ve and (B) affinity matured mAb F(ab′)₂ fragments (100 and 50 nM in (A) and (B), respectively) to the IgG binding domains SpA-A, B, C, D, E and Sbi-I as described in Example 2.

FIG. 6. Improved binding response (A) and affinity (B) of affinity matured anti-SpA mAb F(ab′)₂ and Fab fragments (100 nM), as described in Example 2.

FIG. 7. Binding of anti-SpA Fab and F(ab′)₂ fragments to live S. aureus as described in Example 3.

FIG. 8. Specific binding of anti-SpA F(ab′)₂ fragments to live S. aureus wt but not to AspAAsbi as described in Example 3.

FIG. 9. Improved binding of affinity matured offspring anti-SpA F(ab′)₂ fragments to the surface of live S. aureus cells relative to parent lineage as described in Example 3.

FIG. 10. Inhibition of non-VH3 human IgG (66.7 μM) binding to SpA by anti-SpA F(ab′)₂ fragments (100 nM) by fortéBio analysis as described in Example 4.

FIG. 11. Inhibition of human IgG binding to S. aureus by anti-SpA F(ab′)₂ in flow cytometry based assay as described in Example 4.

FIG. 12. Binding of directly labeled anti-SpA full IgGs to S. aureus in the presence of human serum IgGs as described in Example 5.

FIG. 13. Increased binding of affinity improved anti-SpA full IgGs to S. aureus in the presence of human serum IgGs as described in Example 5.

FIG. 14. Specific SpA dependent killing of S. aureus in a PMN opsonophagocytosis assay as described in Example 6.

FIG. 15. Protection by anti-SpA mAbs in S. aureus induced murine bacteremia/sepsis models as described in Example 7.

### DETAILED DESCRIPTION

The term “antibody” as used herein shall refer to polypeptides or proteins that consist of or comprise antibody domains, which are understood as constant and/or variable domains of the heavy and/or light chains of immunoglobulins, with or without a linker sequence. Polypeptides are understood as antibody domains, if comprising a beta-barrel structure consisting of at least two beta-strands of an antibody domain structure connected by a loop sequence. Antibody domains may be of native structure or modified by mutagenesis or derivatization, e.g. to modify the antigen binding properties or any other property, such as stability or functional properties, such as binding to the Fc receptors FcRn and/or Fcgamma receptor.

The antibody as used herein has a specific binding site to bind one or more antigens or one or more epitopes of such antigens, specifically comprising a CDR binding site of a single variable antibody domain, such as VH, VL or VHH, or a binding site of pairs of variable antibody domains, such as a VLNH pair, an antibody comprising a VLNH domain pair and constant antibody domains, such as Fab, F(ab′), (Fab)₂, scFv, Fv, or a full length antibody.

The term “antibody” as used herein shall particularly refer to antibody formats comprising or consisting of single variable antibody domain, such as VH, VL or VHH, or combinations of variable and/or constant antibody domains with or without a linking sequence or hinge region, including pairs of variable antibody domains, such as a VLNH pair, an antibody comprising or consisting of a VLNH domain pair and constant antibody domains, such as heavy-chain antibodies, Fab, F(ab′), (Fab)₂, scFv, Fd, Fv, or a full-length antibody, e.g. of an IgG type (e.g., an IgG1, IgG2, IgG3, or IgG4 sub-type), IgA1, IgA2, IgD, IgE, or IgM antibody. The term “full length antibody” can be used to refer to any antibody molecule comprising at least most of the Fc domain and other domains commonly found in a naturally occurring antibody monomer. This phrase is used herein to emphasize that a particular antibody molecule is not an antibody fragment.

The term “antibody” shall specifically include antibodies in the isolated form, which are substantially free of other antibodies directed against different target antigens or comprising a different structural arrangement of antibody domains. Still, an isolated antibody may be comprised in a combination preparation, containing a combination of the isolated antibody, e.g. with at least one other antibody, such as monoclonal antibodies or antibody fragments having different specificities.

The term “antibody” shall apply to antibodies of animal origin, including human species, such as mammalian, including human, murine, rabbit, goat, lama, cow and horse, or avian, such as hen, which term shall particularly include recombinant antibodies which are based on a sequence of animal origin, e.g. human sequences.

The term “antibody” further applies to chimeric antibodies with sequences of origin of different species, such as sequences of murine and human origin.

The term “chimeric” as used with respect to an antibody refers to those antibodies wherein one portion of each of the amino acid sequences of heavy and light chains is homologous to corresponding sequences in antibodies derived from a particular species or belonging to a particular class, while the remaining segment of the chain is homologous to corresponding sequences in another species or class. Typically the variable region of both light and heavy chains mimics the variable regions of antibodies derived from one species of mammals, while the constant portions are homologous to sequences of antibodies derived from another. For example, the variable region can be derived from presently known sources using readily available B-cells or hybridomas from non-human host organisms in combination with constant regions derived from, for example, human cell preparations.

The term “antibody” may further apply to humanized antibodies.

The term “humanized” as used with respect to an antibody refers to a molecule having an antigen binding site that is substantially derived from an immunoglobulin from a non-human species, wherein the remaining immunoglobulin structure of the molecule is based upon the structure and/or sequence of a human immunoglobulin. The antigen binding site may either comprise complete variable domains fused onto constant domains or only the complementarity determining regions (CDR) grafted onto appropriate framework regions in the variable domains. Antigen-binding sites may be wild-type or modified, e.g. by one or more amino acid substitutions, preferably modified to resemble human immunoglobulins more closely. Some forms of humanized antibodies preserve all CDR sequences (for example a humanized mouse antibody which contains all six CDRs from the mouse antibody). Other forms have one or more CDRs which are altered with respect to the original antibody.

The term “antibody” further applies to human antibodies.

The term “human” as used with respect to an antibody, is understood to include antibodies having variable and constant regions derived from human germline immunoglobulin sequences. A human antibody as described herein may include amino acid residues not encoded by human germline immunoglobulin sequences (e.g., mutations introduced by random or site-specific mutagenesis in vitro or by somatic mutation in vivo), for example in the CDRs. Human antibodies include antibodies isolated from human immunoglobulin libraries or from animals transgenic for one or more human immunoglobulin.

The term “antibody” specifically applies to antibodies of any class or subclass. Depending on the amino acid sequence of the constant domain of their heavy chains, antibodies can be assigned to the major classes of antibodies IgA, IgD, IgE, IgG, and IgM, and several of these may be further divided into subclasses (isotypes), e.g., IgG1, IgG2, IgG3, IgG4, IgA1, and IgA2.

The term further applies to monoclonal or polyclonal antibodies, specifically a recombinant antibody, which term includes all antibodies and antibody structures that are prepared, expressed, created or isolated by recombinant means, such as antibodies originating from animals, e.g. mammalians including human, that comprises genes or sequences from different origin, e.g. murine, chimeric, humanized antibodies, or hybridoma derived antibodies. Further examples refer to antibodies isolated from a host cell transformed to express the antibody, or antibodies isolated from a recombinant, combinatorial library of antibodies or antibody domains, or antibodies prepared, expressed, created or isolated by any other means that involve splicing of antibody gene sequences to other DNA sequences.

It is understood that the term “antibody” also refers to derivatives of an antibody, in particular functionally active derivatives. An antibody derivative is understood as any combination of one or more antibody domains or antibodies and/ or a fusion protein, in which any domain of the antibody may be fused at any position of one or more other proteins, such as other antibodies, e.g. a binding structure comprising CDR loops, a receptor polypeptide, but also ligands, scaffold proteins, enzymes, toxins and the like. A derivative of the antibody may be obtained by association or binding to other substances by various chemical techniques such as covalent coupling, electrostatic interaction, di-sulphide bonding etc. The other substances bound to the antibody may be lipids, carbohydrates, nucleic acids, organic and inorganic molecules or any combination thereof (e.g. PEG, prodrugs or drugs). In a specific embodiment, the antibody is a derivative comprising an additional tag allowing specific interaction with a biologically acceptable compound. There is not a specific limitation with respect to the tag usable in the present invention, as far as it has no or tolerable negative impact on the binding of the antibody to its target. Examples of suitable tags include His-tag, Myc-tag, FLAG-tag, Strep-tag, Calmodulin-tag, GST-tag, MBP-tag, and S-tag. In another specific embodiment, the antibody is a derivative comprising a label. The term “label” as used herein refers to a detectable compound or composition which is conjugated directly or indirectly to the antibody so as to generate a “labeled” antibody. The label may be detectable by itself, e.g. radioisotope labels or fluorescent labels, or, in the case of an enzymatic label, may catalyze chemical alteration of a substrate compound or composition which is detectable.

The preferred derivatives as described herein are functionally active with regard to the antigen binding, and alike the antibodies that are not derivatized, preferably have a potency to neutralize S. aureus and/or which are protective antibodies.

Antibodies derived from a parent antibody or antibody sequence, such as a parent CDR or FR sequence, are herein particularly understood as mutants or variants obtained by e.g. in silico or recombinant engineering or else by chemical derivatization or synthesis.

Specifically, an antibody derived from an antibody as described herein may comprise at least one or more of the CDR regions or CDR variants thereof, e.g. at least 3 CDRs of the heavy chain variable region and/or at least 3 CDRs of the light chain variable region, with at least one point mutation in at least one of the CDR or FR regions, or in the constant region of the HC or LC, being functionally active, e.g. determined by essentially the same or improved binding characteristics to the target antigen.

It is understood that the term “antibody” also refers to variants of an antibody, including antibodies comprising functionally active CDR variants of a parent CDR sequence, and functionally active variant antibodies of a parent antibody.

The term “variant” shall particularly refer to antibodies, such as mutant antibodies or fragments of antibodies, e.g. obtained by mutagenesis methods, in particular to delete, exchange, introduce inserts into a specific antibody amino acid sequence or region or chemically derivatise an amino acid sequence, e.g. in the constant domains to engineer the antibody stability, effector function or half-life, or in the variable domains to improve antigen-binding properties, e.g. by affinity maturation techniques available in the art. Any of the known mutagenesis methods may be employed, including point mutations at desired positions, e.g. obtained by randomisation techniques. In some cases positions are chosen randomly, e.g. with either any of the possible amino acids or a selection of preferred amino acids to randomise the antibody sequences. The term “mutagenesis” refers to any art recognized technique for altering a polynucleotide or polypeptide sequence. Preferred types of mutagenesis include error prone PCR mutagenesis, saturation mutagenesis, or other site directed mutagenesis.

The term “variant” shall specifically encompass functionally active variants.

The term “functionally active variant” of a CDR sequence as used herein, is understood as a “functionally active CDR variant”, and the “functionally active variant” of an antibody as used herein, is understood as “functionally active antibody variant”. The functionally active variant means a sequence resulting from modification of this sequence (a parent antibody or a parent sequence) by insertion, deletion or substitution of one or more amino acids, or chemical derivatization of one or more amino acid residues in the amino acid sequence, or nucleotides within the nucleotide sequence, or at either or both of the distal ends of the sequence, e.g. in a CDR sequence the N-terminal and/or C-terminal 1, 2, 3, or 4 amino acids, and/or the centric 1, 2, 3, or 4 amino acids (i.e. in the midst of the CDR sequence), and which modification does not affect, in particular impair, the activity of this sequence. In the case of a binding site having specificity to a selected target antigen, the functionally active variant of an antibody would still have the predetermined binding specificity, though this could be changed, e.g. to change the fine specificity to a specific epitope, the affinity, the avidity, the Kon or Koff rate, etc. For example, an affinity matured antibody is specifically understood as a functionally active variant antibody. Hence, the modified CDR sequence in an affinity matured antibody is understood as a functionally active CDR variant. Further modifications may be made through mutagenesis, e.g. to widen the cross-specificity to target more IGBP domains (e.g. different types of domains or domain variants originating from different strains) than the parent antibody, or to increase its reactivity with one or more of the targets. A specific indicator of functional activity is considered the competitive binding to inhibit binding of any of the IGBP to the natural ligand, i.e. the IgG-Fc of serum immunoglobulins.

Preferred antibodies as described herein are binding the individual target antigens (the individual IGBP domains) with a high affinity, in particular with a high on and/or a low off rate, or a high avidity of binding. The binding affinity of an antibody is usually characterized in terms of the concentration of the antibody, at which half of the antigen binding sites are occupied, known as the dissociation constant (Kd, or K_{D}). Usually a binder is considered a high affinity binder with a K_{D}<10⁻⁸ M, preferably a K_{D}<5×10⁻⁹ M, even more preferred is a K_{D}<10⁻⁹ M, or K_{D}<5×10⁻¹⁰ M, or K_{D}<10⁻¹⁰ M.

Yet, in a particularly preferred embodiment the individual antigen binding affinities are of medium affinity, e.g. with a K_{D} of less than 10⁻⁶ M and up to 10⁻⁸ M, e.g. when binding to at least two antigens.

Medium affinity binders may be provided according to the invention, preferably in conjunction with an affinity maturation process, if necessary.

Affinity maturation is the process by which antibodies with increased affinity for a target antigen are produced. Any one or more methods of preparing and/or using affinity maturation libraries available in the art may be employed in order to generate affinity matured antibodies in accordance with various embodiments of the invention disclosed herein. Exemplary such affinity maturation methods and uses, such as random mutagenesis, bacterial mutator strains passaging, site-directed mutagenesis, mutational hotspots targeting, parsimonious mutagenesis, antibody shuffling, light chain shuffling, heavy chain shuffling, CDR1 and/or CDR1 mutagenesis, and methods of producing and using affinity maturation libraries amenable to implementing methods and uses in accordance with various embodiments of the invention disclosed herein, include, for example, those disclosed in: Prassler et al. (2009); Immunotherapy, Vol. 1(4), pp. 571-583; Sheedy et al. (2007), Biotechnol. Adv., Vol. 25(4), pp. 333-352; WO2012/009568; WO2009/036379; WO2010/105256; US2002/0177170; WO2003/074679.

With structural changes of an antibody, including amino acid mutagenesis or as a consequence of somatic mutation in immunoglobulin gene segments, variants of a binding site to an antigen are produced and selected for greater affinities. Affinity matured antibodies may exhibit a several logfold greater affinity than a parent antibody. Single parent antibodies may be subject to affinity maturation. Alternatively pools of antibodies with similar binding affinity to the target antigen may be considered as parent structures that are varied to obtain affinity matured single antibodies or affinity matured pools of such antibodies.

The preferred affinity maturated variant of an antibody as described herein exhibits at least a 2 fold increase in affinity of binding, preferably at least a 5, preferably at least 10, preferably at least 50, or preferably at least 100 fold increase. The affinity maturation may be employed in the course of the selection campaigns employing respective libraries of parent molecules, either with antibodies having medium binding affinity to obtain the antibody of the invention having the specific target binding property of a binding affinity K_{D}<10⁻⁸ M. Alternatively, the affinity may be even more increased by affinity maturation of the antibody according to the invention to obtain the high values corresponding to a K_{D} of less than 10⁻⁹ M, preferably less than 10⁻¹⁰ M or even less than 10⁻¹¹ M, most preferred in the picomolar range.

In certain embodiments binding affinity is determined by an affinity ELISA assay. In certain embodiments binding affinity is determined by a BlAcore, ForteBio or MSD assays. In certain embodiments binding affinity is determined by a kinetic method. In certain embodiments binding affinity is determined by an equilibrium/solution method.

The functional activity is preferably determined in an assay for determining the opsoniphagocytc killing activity of antibodies, e.g. determined in a standard OPK assay by measuring decreased viability or bacterial cells. Specific tests for determining protection are in vitro OPK assays or in vivo efficacy testing in murine bacteremia or sepsis model, such as described in the examples section.

Typically the functional activity of variants is proven if they exhibit substantially the same functional activity or substantially the same biological activity as the comparable (parent or non-modified) antibody.

The term “substantially the same functional activity” or “substantially the same biological activity” as used herein refers to the activity as indicated by substantially the same activity being at least 20%, at least 50%, at least 75%, at least 90%, e.g. at least 100%, or at least 125%, or at least 150%, or at least 175%, or e.g. up to 200% of the activity as determined for the comparable or parent antibody.

The term “substantially the same binding affinity” with regard to binding a wild-type or mutant IGBP domains, e.g. the IGBP_{KK} or IGBP_{KKAA} domain, is understood as the affinity represented by a K_{D} as determined in the same assay, e.g. using the same type of antibody and the same type of antigen to produce comparable results of koff and kon and K_{D}, respectively, wherein the K_{D} difference is less than 2-fold, or less than 4-fold.

According to the examples provided herein, the affinity measurement is performed as follows: Affinity measurements are performed by interferometry using a recombinant IGBP domain as antigen, and the antibody is produced as F(ab′)₂ or F(ab) fragments to determine the affinity of binding the antigen by the CDR binding site. The F(ab′)₂ or F(ab) fragments are expressed by a recombinant host and optionally further purified to avoid contaminating substances which could interfere with the affinity measurement. If an antibody is produced as IgG and further digested by pepsin to obtain the F(ab′)₂ preparation, the F(ab′)₂ preparation is optionally purified to avoid contaminating Fc fragments which could interfere with the affinity measurement.

According to the specific examples provided herein, affinity measurements were performed by interferometry using a ForteBio Octet Red instrument [Pall Life Sciences]; the biotinylated antigen was immobilized on streptavidin sensors to give a sensor loading of ˜2 nm. The association and dissociation of the antibody F(ab′)₂ or F(ab) fragments (50-100 and 100-200 nM, respectively), in solution (PBS, pH 7.2 plus 1% BSA), were measured at 30° C. for 3-10 min for the association phase and 3-30 min for the dissociation phase. The dissociation constants (K_{D} values) were calculated based on the kinetic parameters (kon and koff) determined by fitting simultaneously the association and dissociation phases to a 1:1 binding model using Octet Data Analysis Software version 7. Biotinylated SpA and Sbi domains (SpA-A, SpA-B, SpA-C, SpA-D, SpA-E, Sbi-I and Sbi-II) were produced as described in Example 1 and F(ab′)₂ fragments were generated from yeast and or CHO derived IgGs by pepsin digestion as described in Example 2.

The preferred variants or derivatives as described herein are functionally active with regard to the antigen binding, preferably which have a potency to specifically bind the individual targets, and not significantly binding to other antigens that are not target antigens, e.g. with a K_{D} value difference of at least 2 logs, preferably at least 3 logs. The antigen binding by a functionally active variant is typically not impaired, corresponding to about substantially the same binding affinity as the parent antibody or sequence, or antibody comprising a sequence variant, e.g. with a a K_{D} value difference of less than 2 logs, preferably less than 3 logs, however, with the possibility of even improved affinity, e.g. with a K_{D} value difference of at least 1 log, preferably at least 2 logs.

In a preferred embodiment the functionally active variant of a parent antibody

a) is a biologically active fragment of the antibody, the fragment comprising at least 50% of the sequence of the molecule, preferably at least 60%, at least 70%, at least 80%, at least 90%, or at least 95% and most preferably at least 97%, 98% or 99%;

b) is derived from the antibody by at least one amino acid substitution, addition and/or deletion, wherein the functionally active variant has a sequence identity to the molecule or part of it, such as an antibody of at least 50% sequence identity, preferably at least 60%, more preferably at least 70%, more preferably at least 80%, still more preferably at least 90%, even more preferably at least 95% and most preferably at least 97%, 98% or 99%; and/or

c) consists of the antibody or a functionally active variant thereof and additionally at least one amino acid or nucleotide heterologous to the polypeptide or the nucleotide sequence.

In one preferred embodiment of the invention, the functionally active variant of the antibody according to the invention is essentially identical to the variant described above, but differs from its polypeptide or the nucleotide sequence, respectively, in that it is derived from a homologous sequence of a different species. These are referred to as naturally occurring variants or analogs.

The term “functionally active variant” also includes naturally-occurring allelic variants, as well as mutants or any other non-naturally occurring (e.g. synthetic or artificial) antibodies, or variants, such as those comprising antigen-binding sequences derived from artificial antibody libraries. As is known in the art, an allelic variant is an alternate form of a (poly)peptide that is characterized as having a substitution, deletion, or addition of one or more amino acids that does essentially not alter the biological function of the polypeptide.

Functionally active variants may be obtained by sequence alterations in the polypeptide or the nucleotide sequence, e.g. by one or more point mutations, wherein the sequence alterations retains or improves a function of the unaltered polypeptide or the nucleotide sequence, when used in combination of the invention. Such sequence alterations can include, but are not limited to, (conservative) substitutions, additions, deletions, mutations and insertions.

Specific functionally active variants are CDR variants. A CDR variant includes an amino acid sequence modified by at least one amino acid in the CDR region, wherein said modification can be a chemical or a partial alteration of the amino acid sequence, which modification permits the variant to retain the biological characteristics of the unmodified sequence. A partial alteration of the CDR amino acid sequence may be by deletion or substitution of one to several amino acids, e.g. 1, 2, 3, 4 or 5 amino acids, or by addition or insertion of one to several amino acids, e.g. 1, 2, 3, 4 or 5 amino acids, or by a chemical derivatization of one to several amino acids, e.g. 1, 2, 3, 4 or 5 amino acids, or combination thereof. The substitutions in amino acid residues may be conservative substitutions, for example, substituting one hydrophobic amino acid for an alternative hydrophobic amino acid.

Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc.

A point mutation is particularly understood as the engineering of a poly-nucleotide that results in the expression of an amino acid sequence that differs from the non-engineered amino acid sequence in the substitution or exchange, deletion or insertion of one or more single (non-consecutive) or doublets of amino acids for different amino acids.

Preferred point mutations refer to the exchange of amino acids of the same polarity and/or charge. In this regard, amino acids refer to twenty naturally occurring amino acids encoded by sixty-four triplet codons. These 20 amino acids can be split into those that have neutral charges, positive charges, and negative charges:

The “neutral” amino acids are shown below along with their respective three-letter and single-letter code and polarity:

Alanine: (Ala, A) nonpolar, neutral;

Asparagine: (Asn, N) polar, neutral;

Cysteine: (Cys, C) nonpolar, neutral;

Glutamine: (Gln, Q) polar, neutral;

Glycine: (Gly, G) nonpolar, neutral;

Isoleucine: (Ile, I) nonpolar, neutral;

Leucine: (Leu, L) nonpolar, neutral;

Methionine: (Met, M) nonpolar, neutral;

Phenylalanine: (Phe, F) nonpolar, neutral;

Proline: (Pro, P) nonpolar, neutral;

Serine: (Ser, S) polar, neutral;

Threonine: (Thr, T) polar, neutral;

Tryptophan: (Trp, W) nonpolar, neutral;

Tyrosine: (Tyr, Y) polar, neutral;

Valine: (Val, V) nonpolar, neutral; and

Histidine: (His, H) polar, positive (10%) neutral (90%).

The “positively” charged amino acids are:

Arginine: (Arg, R) polar, positive; and

Lysine: (Lys, K) polar, positive.

The “negatively” charged amino acids are:

Aspartic acid: (Asp, D) polar, negative; and

Glutamic acid: (Glu, E) polar, negative.

“Percent (%) amino acid sequence identity” with respect to the antibody sequences and homologs described herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

An antibody variant is specifically understood to include homologs, analogs, fragments, modifications or variants with a specific glycosylation pattern, e.g. produced by glycoengineering, which are functional and may serve as functional equivalents, e.g. binding to the specific targets and with functional properties. The preferred variants as described herein are functionally active with regard to the antigen binding, preferably which have a potency to neutralize S. aureus and/or which are protective antibodies.

An antibody as described herein may or may not exhibit Fc effector function. Though the mode of action is mainly mediated by neutralizing antibodies without Fc effector functions, Fc can recruit complement and aid elimination of the target antigen, such as a toxin, from the circulation via formation of immune complexes.

Specific antibodies may be devoid of an active Fc moiety, thus, either composed of antibody domains that do not contain an Fc part of an antibody or that do not contain an Fcgamma receptor binding site, or comprising antibody domains lacking Fc effector function, e.g. by modifications to reduce Fc effector functions, in particular to abrogate or reduce ADCC and/or CDC activity. Alternative antibodies may be engineered to incorporate modifications to increase Fc effector functions, in particular to enhance ADCC and/or CDC activity.

Such modifications may be effected by mutagenesis, e.g. mutations in the Fcgamma receptor binding site or by derivatives or agents to interfere with ADCC and/or CDC activity of an antibody format, so to achieve reduction or increase of Fc effector function.

A significant reduction of Fc effector function is typically understood to refer to Fc effector function of less than 10% of the unmodified (wild-type) format, preferably less than 5%, as measured by ADCC and/or CDC activity.

A significant increase of Fc effector function is typically understood to refer to an increase in Fc effector function of at least 10% of the unmodified (wild-type) format, preferably at least 20%, 30%, 40% or 50%, as measured by ADCC and/or CDC activity.

The term “glycoengineered” variants with respect to antibody sequences shall refer to glycosylation variants having modified immunogenic or immunomodulatory (e.g. anti-inflammatory) properties, ADCC and/ or CDC, as a result of the glycoengineering. All antibodies contain carbohydrate structures at conserved positions in the heavy chain constant regions, with each isotype possessing a distinct array of N-linked carbohydrate structures, which variably affect protein assembly, secretion or functional activity. IgG1 type antibodies are glycoproteins that have a conserved N linked glycosylation site at Asn297 in each CH2 domain. The two complex bi-antennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC). Removal of N-Glycan at N297, e.g. through mutating N297, e.g. to A, or T299 typically results in aglycosylated antibody formats with reduced ADCC. Specifically, an antibody as described herein may be glycosylated or glycoengineered, or aglycosylated antibodies.

Major differences in antibody glycosylation occur between cell lines, and even minor differences are seen for a given cell line grown under different culture conditions. Expression in bacterial cells typically provides for an aglycosylated antibody. CHO cells with tetracycline-regulated expression of β(1 ,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180). In addition to the choice of host cells, factors that affect glycosylation during recombinant production of antibodies include growth mode, media formulation, culture density, oxygenation, pH, purification schemes and the like.

The term “antigen-binding site” or “binding site” refers to the part of an antibody that participates in antigen binding. The antigen binding site is formed by amino acid residues of the N-terminal variable (“V”) regions of the heavy (“H”) and/or light (“L”) chains, or the variable domains thereof. Three highly divergent stretches within the V regions of the heavy and light chains, referred to as “hypervariable regions”, are interposed between more conserved flanking stretches known as framework regions, The antigen-binding site provides for a surface that is complementary to the three-dimensional surface of a bound epitope or antigen, and the hypervariable regions are referred to as “complementarity-determining regions”, or “CDRs.” The binding site incorporated in the CDRs is herein also called “CDR binding site”.

Specifically, the CDR sequences as referred to herein are understood as those amino acid sequences of an antibody as determined according to Kabat nomenclature (see Kabat et al., Sequences of Proteins of Immunological Interest, 5^{th} Ed. Public Health Service, U.S. Department of Health and Human Services. (1991)).

The term “antigen” as used herein interchangeably with the terms “target” or “target antigen” shall refer to a whole target molecule or a fragment of such molecule recognized by an antibody binding site. Specifically, substructures of an antigen, e.g. a polypeptide or carbohydrate structure, generally referred to as “epitopes”, e.g. B-cell epitopes or T-cell epitope, which are immunologically relevant, may be recognized by such binding site.

The term “epitope” as used herein shall in particular refer to a molecular structure which may completely make up a specific binding partner or be part of a specific binding partner to a binding site of an antibody. An epitope may either be composed of a carbohydrate, a peptidic structure, a fatty acid, an organic, biochemical or inorganic substance or derivatives thereof and any combinations thereof. If an epitope is comprised in a peptidic structure, such as a peptide, a polypeptide or a protein, it will usually include at least 3 amino acids, preferably 5 to 40 amino acids, and more preferably between about 10-20 amino acids. Epitopes can be either linear or conformational epitopes. A linear epitope is comprised of a single segment of a primary sequence of a polypeptide or carbohydrate chain. Linear epitopes can be contiguous or overlapping.

Conformational epitopes are comprised of amino acids or carbohydrates brought together by folding the polypeptide to form a tertiary structure and the amino acids are not necessarily adjacent to one another in the linear sequence. Specifically and with regard to polypeptide antigens a conformational or discontinuous epitope is characterized by the presence of two or more discrete amino acid residues, separated in the primary sequence, but assembling to a consistent structure on the surface of the molecule when the polypeptide folds into the native protein/antigen. Specifically, the conformational epitope is an epitope which is comprised of a series of amino acid residues which are non-linear in alignment that is that the residues are spaced or grouped in a non-continuous manner along the length of a polypeptide sequence.

Such conformational epitope is characterized by a three-dimensional structure with specific structure coordinates as determined by contacting amino acid residues and/or crystallographic analysis, e.g. analysis of a crystal formed by the immune complex of the epitope bound by a specific antibody or Fab fragment.

Herein the term “epitope” shall particularly refer to the single epitope recognized by an antibody, or a series of epitope variants, each recognized by a cross-reactive antibody.

A specific cross-reactive epitope as targeted by an antibody of the invention may be incorporated in the consensus sequence of SEQ ID 145, or interfere with binding of a natural ligand to such consensus sequence.

Thus, an anti-IGBP antibody as described herein may bind to the wild-type IGBP at least substantially to the same extent as to the mutant IGBP_{KK} or IGBP_{KKAA}. Specifically, anti-IGBP antibody as described herein may preferentially bind to the wild-type IGBP, e.g. preferentially binding to such consensus sequence of SEQ ID 145 (of the wild-type IGBP, included in each of the SpA and Sbi domains), and bind to the sequence of a mutant IGBP domain only to a less extent.

The IGBP mutant designated IGBP_{KK} (e.g. SpA-A_{KK}, SpA-B_{KK}, SpA-C_{KK}, SpA-D_{KK}, SpA-E_{KK}, Sbi-I_{KK}, SBi-II_{KK}) comprises the following sequence:

SEQ ID 147
KKXAFYXXL

Wherein

X at position 3 is any of N, S, or K

X at position 7 is any of E, Q, or N, and

X at position 8 is any of I or V.

The IGBPmutant designated IGBP_{KKAA} (e.g. SpA-A_{KKAA}, SpA-B_{KKAA}, SPA-C_{KKAA}, SPA-D_{KKAA}, SpA-E_{KKAA}) comprises the sequences SEQ ID 147, and further comprises SEQ ID 148:

SEQ ID 148
QRNGFIQSLKAAPSXS

Wherein

X at position 15 is any of Q or V.

The respective wild-type consensus sequence comprised in each of the SpA-A, SpA-B, SpA-C, SpA-D, and SpA-E is as follows (SEQ ID 146):

SEQ ID 146
QRNGFIQSLKDDPSXS

Wherein

X at position 15 is any of Q or V.

The invention specifically employs cross-reactive antibodies, which are obtained by a process to identify neutralizing antibodies with multiple specificities, e.g. by a cross-reactive discovery selection scheme. Accordingly, an antibody library including antibodies showing reactivity with two targets, targets A and B, may first be selected for reactivity with one of the targets, e.g. target A, followed by selection for reactivity with the other target, e.g. target B. Each successive selection round reinforces the reactive strength of the resulting pool towards both targets. Accordingly, this method is particularly useful for identifying antibodies with cross-reactivity directed to the two different targets, and the potential to cross-neutralize a pathogen. The method can be extended to identifying antibodies showing reactivity towards further targets, by including additional rounds of enrichment towards the additional target(s).

Cross-reactive antibodies, in some instances, emerge through screening against single antigens. To increase the likelihood of isolating cross-reactivity clones one would apply multiple selective pressures by processively screening against multiple antigens. Special mAb selection strategies employ the different toxin components or different toxin variants, or different IGBP domains in an alternating fashion.

For example, according to a unique approach, high affinity fully human monoclonal antibodies are selected form a respective antibody library using wild type (unmutated) IgG binding domain sequences and in vitro expression library of human IgGs lacking the VH3 sub-family.

The recombinant IGBP domains produced by recombinant techniques employing the respective sequences as provided in FIG. 2, may be used for selecting antibodies from an antibody library, e.g. a yeast platform derived antibody library see, for example: Blaise L, Wehnert A, Steukers M P, van den Beucken T, Hoogenboom H R, Hufton S E. Construction and diversification of yeast cell surface displayed libraries by yeast mating: application to the affinity maturation of Fab antibody fragments. Gene. 2004 Nov. 24; 342(2):211-8; Boder E T, Wittrup K D. Yeast surface display for screening combinatorial polypeptide libraries. Nat Biotechnol. 1997 June; 15(6):553-7; Kuroda K, Ueda M. Cell surface engineering of yeast for applications in white biotechnology. Biotechnol Lett. 2011 January; 33(1):1-9. doi: 10.1007/s10529-010-0403-9. Review; Lauer T M, Agrawal N J, Chennamsetty N, Egodage K, Helk B, Trout B L. Developability index: a rapid in silico tool for the screening of antibody aggregation propensity. J Pharm Sci. 2012 January; 101(1):102-15; Orcutt K. D. and Wittrup K. D. (2010), 207-233 doi: 10.1007/978-3-642-01144-3_15; Rakestraw J A, Aird D, Aha P M, Baynes B M, Lipovsek D. Secretion-and-capture cell-surface display for selection of target-binding proteins. Protein Eng Des Sel. 2011 June; 24(6):525-30; U.S. Pat. No. 6,423,538; U.S. Pat. No. 6,696,251; U.S. Pat. No. 6,699,658; published PCT application publication No. WO2008118476.

In either event, reactivity or cross-reactivity can be further improved by antibody optimization methods known in the art. For example, certain regions of the variable regions of the immunoglobulin chains described herein may be subjected to one or more optimization strategies, including light chain shuffling, destinational mutagenesis, CDR amalgamation, and directed mutagenesis of selected CDR and/or framework regions.

Screening methods for identifying antibodies with the desired neutralizing properties may be opsonophagocytic activity, inhibition of IGBP binding to the target immunoglobulins, or inhibition of in vivo effect on animals (death, hemolysis, overshooting inflammation, organ dysfunction).

Once reactivity or cross-neutralizing antibodies with the desired properties have been identified, the dominant epitope or epitopes recognized by the antibodies may be determined. Methods for epitope mapping are well-known in the art and are disclosed, for example, in Epitope Mapping: A Practical Approach, Westwood and Hay, eds., Oxford University Press, 2001.

The term “expression” is understood in the following way. Nucleic acid molecules containing a desired coding sequence of an expression product such as e.g. an antibody as described herein, and control sequences such as e.g. a promoter in operable linkage, may be used for expression purposes. Hosts transformed or transfected with these sequences are capable of producing the encoded proteins. In order to effect transformation, the expression system may be included in a vector; however, the relevant DNA may also be integrated into the host chromosome. Specifically the term refers to a host cell and compatible vector under suitable conditions, e.g. for the expression of a protein coded for by foreign DNA carried by the vector and introduced to the host cell.

Coding DNA is a DNA sequence that encodes a particular amino acid sequence for a particular polypeptide or protein such as e.g. an antibody. Promoter DNA is a DNA sequence which initiates, regulates, or otherwise mediates or controls the expression of the coding DNA. Promoter DNA and coding DNA may be from the same gene or from different genes, and may be from the same or different organisms. Recombinant cloning vectors will often include one or more replication systems for cloning or expression, one or more markers for selection in the host, e.g. antibiotic resistance, and one or more expression cassettes.

“Vectors” used herein are defined as DNA sequences that are required for the transcription of cloned recombinant nucleotide sequences, i.e. of recombinant genes and the translation of their mRNA in a suitable host organism.

An “expression cassette” refers to a DNA coding sequence or segment of DNA that code for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”.

Expression vectors comprise the expression cassette and additionally usually comprise an origin for autonomous replication in the host cells or a genome integration site, one or more selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together. The term “vector” as used herein includes autonomously replicating nucleotide sequences as well as genome integrating nucleotide sequences. A common type of vector is a “plasmid”, which generally is a self-contained molecule of double-stranded DNA that can readily accept additional (foreign) DNA and which can readily be introduced into a suitable host cell. A plasmid vector often contains coding DNA and promoter DNA and has one or more restriction sites suitable for inserting foreign DNA. Specifically, the term “vector” or “plasmid” refers to a vehicle by which a DNA or RNA sequence (e.g. a foreign gene) can be introduced into a host cell, so as to transform the host and promote expression (e.g. transcription and translation) of the introduced sequence.

The term “host cell” as used herein shall refer to primary subject cells transformed to produce a particular recombinant protein, such as an antibody as described herein, and any progeny thereof. It should be understood that not all progeny are exactly identical to the parental cell (due to deliberate or inadvertent mutations or differences in environment), however, such altered progeny are included in these terms, so long as the progeny retain the same functionality as that of the originally transformed cell. The term “host cell line” refers to a cell line of host cells as used for expressing a recombinant gene to produce recombinant polypeptides such as recombinant antibodies. The term “cell line” as used herein refers to an established clone of a particular cell type that has acquired the ability to proliferate over a prolonged period of time. Such host cell or host cell line may be maintained in cell culture and/or cultivated to produce a recombinant polypeptide.

Antibodies or antibody fragments can be produced by methods well-known in the art, including, for example, hybridoma techniques or recombinant DNA technology.

In the hybridoma method, a mouse or other appropriate host animal, such as a hamster, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell.

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

Recombinant monoclonal antibodies can, for example, be produced by isolating the DNA encoding the required antibody chains and transfecting a recombinant host cell with the coding sequences for expression, using well known recombinant expression vectors, e.g. the plasmids of the invention or expression cassette(s) comprising the nucleotide sequences encoding the antibody sequences. Recombinant host cells can be prokaryotic and eukaryotic cells, e.g. including animal or human cell lines in cell cultures.

According to a specific aspect, a coding nucleotide sequence may be used for genetic manipulation to humanize the antibody or to improve the affinity, or other characteristics of the antibody. For example, the constant region may be engineered to more nearly resemble human constant regions to avoid immune response, if the antibody is used in clinical trials and treatments in humans. It may be desirable to genetically manipulate the antibody sequence to obtain greater affinity to the target toxins and greater efficacy against S. aureus. It will be apparent to one of skill in the art that one or more polynucleotide changes can be made to the antibody and still maintain its binding ability to the target antigens.

The production of antibody molecules, by various means, is generally well understood. U.S. Pat. No. 6,331,415 (Cabilly et al.), for example, describes a method for the recombinant production of antibodies where the heavy and light chains are expressed simultaneously from a single vector or from two separate vectors in a single cell. Wibbenmeyer et al., (1999, Biochim Biophys Acta 1430(2):191-202) and Lee and Kwak (2003, J. Biotechnology 101 :189-198) describe the production of monoclonal antibodies from separately produced heavy and light chains, using plasmids expressed in separate cultures of E. coli. Various other techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988).

In a specific aspect, the invention provides an isolated nucleic acid comprising a sequence that codes for production of the recombinant antibody of the present invention.

An antibody encoding nucleic acid can have any suitable characteristics and comprise any suitable features or combinations thereof. Thus, for example, an antibody encoding nucleic acid may be in the form of DNA, RNA, or a hybrid thereof, and may include non-naturally-occurring bases, a modified backbone, e.g., a phosphorothioate backbone that promotes stability of the nucleic acid, or both. The nucleic acid advantageously may be incorporated in an expression cassette, vector or plasmid of the invention, comprising features that promote desired expression, replication, and/or selection in target host cell(s). Examples of such features include an origin of replication component, a selection gene component, a promoter component, an enhancer element component, a polyadenylation sequence component, a termination component, and the like, numerous suitable examples of which are known.

The present disclosure further provides the recombinant DNA constructs comprising one or more of the nucleotide sequences described herein. These recombinant constructs are used in connection with a vector, such as a plasmid, phagemid, phage or viral vector, into which a DNA molecule encoding any disclosed antibody is inserted.

If desired, the polynucleotide sequence encoding any of the exemplified antibodies may be cloned into a vector for expression or propagation. The sequence encoding the antibody may be maintained in vector in a host cell and the host cell can then be expanded and frozen for future use. Production of recombinant monoclonal antibodies in cell culture can be carried out through cloning of antibody genes from B cells by means known in the art.

Monoclonal antibodies are typically produced using any method that produces antibody molecules by continuous cell lines in culture. Examples of suitable methods for preparing monoclonal antibodies include the hybridoma methods of Kohler et al. (1975, Nature 256:495-497) and the human B-cell hybridoma method (Kozbor, 1984, J. Immunol. 133:3001; and Brodeur et al., 1987, Monoclonal Antibody Production Techniques and Applications, (Marcel Dekker, Inc., New York), pp. 51-63).

The term “isolated” or “isolation” as used herein with respect to an antibody shall refer to such compound that has been sufficiently separated from the environment with which it would naturally be associated, so as to exist in “substantially pure” form.

“Isolated” does not necessarily mean the exclusion of artificial or synthetic mixtures with other compounds or materials, or the presence of impurities that do not interfere with the fundamental activity, and that may be present, for example, due to incomplete purification. In particular, isolated nucleic acid molecules of the present invention are also meant to include those which are not naturally occurring, e.g. codon-optimized nucleic acids or cDNA, or chemically synthesized.

Likewise, in some embodiments, an isolated antibody of the invention is specifically non-naturally occurring, e.g. as provided in a combination preparation with another antibody, which combination does not occur in nature (such as a combination with one or more monospecific antibody and/or with a cross-specific antibody which recognizes at least two different targets), or an optimized or affinity—matured variant of a naturally occurring antibody, or an antibody with a framework-region which is engineered to improve the manufacturability of the antibody.

With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

With reference to polypeptides or proteins, such as isolated antibodies, the term “isolated” shall specifically refer to compounds that are free or substantially free of material with which they are naturally associated such as other compounds with which they are found in their natural environment, or the environment in which they are prepared (e g. cell culture) when such preparation is by recombinant DNA technology practiced in vitro or in vivo. Isolated compounds can be formulated with diluents or adjuvants and still for practical purposes be isolated - for example, the polypeptides or polynucleotides can be mixed with pharmaceutically acceptable carriers or excipients when used in diagnosis or therapy. The term “isolated antibodies” as used herein is specifically meant to include recombinant antibodies or monoclonal antibodies obtained from cell culture, such as produced by cultivating recombinant host cells that have been transformed with artificial nucleic acid constructs encoding the antibodies, or those chemically synthesized.

The term “IGBP” and “IGBP domains” as used herein is specifically understood as any of the five SpA and the two Sbi domains (SpA-A, SpA-B, SpA-C, SpA-D, SpA-E, Sbi-I, Sbi-II) with a triple helix structure that are able to bind the constant region of IgG (Fc) via conserved residues located on helixes 1 and 2 (Deisenhofer, J. Crystallographic refinement and atomic models of a human Fc fragment and its complex with fragment B of Protein A from S. aureus at 2.9- and 2.8-A resolution, Biochemistry 20, 1981, 2361), with the SpA domains having an additional binding site on helixes 2 and 3, that interacts with the variable region of immunoglobulins with VH3 germline (Graille, M. et al. Crystal structure of a Staphylococcus aureus protein A complexed with the Fab fragment of a human IgM antibody: Structural basis for recognition of B-cell receptors and superantigen activity, Proc. Natl. Acad. Sci. USA 97, 2000, 5399). The avirulent KKAA variants of the IGBP domains show reduced binding to the Fc and VH3 (WO 2014/179744A1, US 2014/0170134).

The term “neutralizing” or “neutralization” is used herein in the broadest sense and refers to any molecule that counteracts a pathogen or inhibits a pathogen, such as S. aureus from infecting a subject, or to inhibit the pathogen from promoting infections by producing potent virulence factors, or to inhibit the virulence factors from exerting its effect in a subject, irrespective of the mechanism by which neutralization is achieved. Neutralization can be achieved, e.g., by an antibody that inhibits the binding and/or interaction of the S. aureus virulence factor(s) with its binding to molecules on target cells or in solution. In certain embodiments, the antibodies described herein can inhibit the virulence factor activity wherein the in vivo or in vitro effects of the interaction between the virulence factor and the host are reduced or eliminated. In the case of IGBP domains, neutralization may occur by allowing the attack by serum IgG binding to surface antigens of S. aureus without interference by the IGBP of S. aureus. Moreover, the antibodies described herein counteract the S. aureus by promoting OPK.

The term “Staphylococcus aureus” or “S. aureus” or “pathogenic S. aureus” is understood in the following way. Staphylococcus aureus bacteria are normally found on the skin or in the nose of people and animals. The bacteria are generally harmless, unless they enter the body through a cut or other wound. Typically, infections are minor skin problems in healthy people. Historically, infections were treated by broad-spectrum antibiotics, such as methicillin. Now, though, certain strains have emerged that are resistant to methicillin and other beta-lactam antibiotics, such as penicillin and cephalosporins. They are referred to as methicillin-resistant Staphylococcus aureus (also known as multi-drug resistant Staphylococcus aureus, or “MRSA”).

Staphylococcus aureus, an important human pathogen, expresses a multitude of secreted toxins (exotoxins). These can attack various host cell types, including erythrocytes, neutrophil granulocytes and other immune cells, as well as epithelial cells of the lung or skin. A prominent member of S. aureus toxins is alpha hemolysin (Hla), which exerts cytolytic function on lymphocytes, macrophages, lung epithelial cells and pulmonary endothelial cells.

S. aureus infections, including MRSA, generally start as small red bumps that resemble pimples, boils or spider bites. These bumps or blemishes can quickly turn into deep, painful abscesses that require surgical draining. Sometimes the bacteria remain confined to the skin. On occasion, they can burrow deep into the body, causing potentially life-threatening infections in a broad range of human tissue, including skin, soft tissue, bones, joints, surgical wounds, the bloodstream, heart valves, lungs, or other organs. Thus, S. aureus infections can result in disease conditions associated therewith, which are potentially fatal diseases, such as necrotizing fasciitis, endocarditis, sepsis, bacteremia, peritonitis, toxic shock syndrome, and various forms of pneumonia, including necrotizing pneumonia, and toxin production in furunculosis and carbunculosis. MRSA infection is especially troublesome in hospital or nursing home settings where patients are at risk of or prone to open wounds, invasive devices, and weakened immune systems and, thus, are at greater risk for infection than the general public.

Antibodies neutralizing S. aureus toxins are interfering with the pathogens and pathogenic reactions, thus able to limit or prevent infection and/ or to ameliorate a disease condition resulting from such infection, or to inhibit S. aureus pathogenesis, in particular pneumonia, peritonitis, osteomyelitis, bacteremia and sepsis pathogenesis. In this regard “protective antibodies” are understood herein as neutralizing antibodies that are responsible for immunity to an infectious agent observed in active or passive immunity. In particular, protective antibodies as described herein are able to neutralize toxic effects (such as cytolysis, induction of pro-inflammatory cytokine expression by target cells) of secreted virulence factors (exotoxins) and hence interfere with pathogenic potential of S. aureus.

The term “recombinant” as used herein shall mean “being prepared by or the result of genetic engineering”. A recombinant host specifically comprises an expression vector or cloning vector, or it has been genetically engineered to contain a recombinant nucleic acid sequence, in particular employing nucleotide sequence foreign to the host. A recombinant protein is produced by expressing a respective recombinant nucleic acid in a host. The term “recombinant antibody”, as used herein, includes antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant antibodies comprise antibodies engineered to include rearrangements and mutations which occur, for example, during antibody maturation.

As used herein, the term “specificity” or “specific binding” refers to a binding reaction which is determinative of the cognate ligand of interest in a heterogeneous population of molecules. Thus, under designated conditions (e.g. immunoassay conditions), an antibody specifically binds to its particular target and does not bind in a significant amount to other molecules present in a sample. The specific binding means that binding is selective in terms of target identity, high, medium or low binding affinity or avidity, as selected. Selective binding is usually achieved if the binding constant or binding dynamics is at least 10 fold different (understood as at least 1 log difference), preferably the difference is at least 100 fold (understood as at least 2 logs difference), and more preferred a least 1000 fold (understood as at least 3 logs difference) as compared to another antigen.

The term “specificity” or “specific binding” is also understood to apply to binders which bind to one or more molecules, e.g. cross-specific binders. Preferred cross-specific (also called polyspecific or cross-reactive) binders targeting at least two different antigens (such as different domains of IGBP), targeting a cross-reactive epitope on at least two different antigens, specifically bind the antigens with substantially similar binding affinity, e.g. with less than 100 fold difference or even less than 10 fold difference.

For example, a cross-specific antibody will be able to bind to the various antigens carrying a cross-reactive epitope. Such binding site of an antibody or and antibody with a specificity to bind at least two different antigens or a cross-reactive epitope of at least two different antigens is also called a polyspecific or cross-specific binding site and antibody, respectively. For example, an antibody may have a polyspecific binding site specifically binding an epitope cross-reactive a number of different antigens with sequence homology within the epitope and/or structural similarities to provide for a conformational epitope of essentially the same structure, e.g. cross-reactive at least the SpA-E domain and two further IGBP domains of different type, or cross-reactive the same type of IGBP domain of at least two different strains.

The immunospecificity of an antibody, its binding capacity and the attendant affinity the antibody exhibits for a cross-reactive binding sequence, are determined by a cross-reactive binding sequence with which the antibody immunoreacts (binds). The cross-reactive binding sequence specificity can be defined, at least in part, by the amino acid residues of the variable region of the heavy chain of the immunoglobulin the antibody and/ or by the light chain variable region amino acid residue sequence.

Use of the term “having the same specificity”, “having the same binding site” or “binding the same epitope” indicates that equivalent antibodies exhibit the same or essentially the same, i.e. similar immunoreaction (binding) characteristics and compete for binding to a pre-selected target binding sequence. The relative specificity of an antibody molecule for a particular target can be relatively determined by competition assays, e.g. as described in Harlow, et al., ANTIBODIES: A LABORATORY MANUAL, Cold Spring Harbor Laboratory Press, Cold Spring Harbor, N.Y., 1988).

In particular, the functional activity of variants is determined by specificity to the target antigen(s), e.g. by binding the same epitope or substantially the same epitope as the respective parent antibody.

Antibodies are said to “bind to the same epitope” or “comprising the same binding site” or have “essentially the same binding” characteristics, if the antibodies cross-compete so that only one antibody can bind to the epitope at a given point of time, i.e. one antibody prevents the binding or modulating effect of the other.

The term “compete” or “cross-compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope, whether to the same, greater, or lesser extent, the antibodies are said to “cross-compete” with each other for binding of their respective epitope(s). Both competing and cross-competing antibodies are encompassed by the present invention.

Competition herein means a greater relative inhibition than about 30% as determined by competition ELISA analysis or by ForteBio analysis. It may be desirable to set a higher threshold of relative inhibition as criteria of what is a suitable level of competition in a particular context, e.g., where the competition analysis is used to select or screen for new antibodies designed with the intended function of the binding of additional or other IGBP domains of S. aureus. Thus, for example, it is possible to set criteria for the competitive binding, wherein at least 40% relative inhibition is detected, or at least 50%, at least 60%, at least 70%, at least 80%, at least 90% or even at least 100%, before an antibody is considered sufficiently competitive.

The term “subject” as used herein shall refer to a warm-blooded mammalian, particularly a human being or a non-human animal. MRSA is a critically important human pathogen that is also an emerging concern in veterinary medicine. It is present in a wide range of non-human animal species. Thus, the term “subject” may also particularly refer to animals including dogs, cats, rabbits, horses, cattle, pigs and poultry. In particular the medical use of the invention or the respective method of treatment applies to a subject in need of prophylaxis or treatment of a disease condition associated with a S. aureus infection. The subject may be a patient at risk of a S. aureus infection or suffering from disease, including early stage or late stage disease. The term “patient” includes human and other mammalian subjects that receive either prophylactic or therapeutic treatment. The term “treatment” is thus meant to include both prophylactic and therapeutic treatment.

A subject is e.g. treated for prophylaxis or therapy of S. aureus disease conditions. In particular, the subject is treated, which is either at risk of infection or developing such disease or disease recurrence, or a subject that is suffering from such infection and/ or disease associated with such infection.

Specifically the term “prophylaxis” refers to preventive measures which is intended to encompass prevention of the onset of pathogenesis or prophylactic measures to reduce the risk of pathogenesis.

Specifically, the method for treating, preventing, or delaying a disease condition in a subject as described herein, is by interfering with the pathogenesis of S. aureus as causal agent of the condition, wherein the pathogenesis includes a step of forming a pore on the subject's cellular membrane, e.g. by the specific virulence factors or toxins.

The term “substantially pure” or “purified” as used herein shall refer to a preparation comprising at least 50% (w/w), preferably at least 60%, 70%, 80%, 90% or 95% of a compound, such as a nucleic acid molecule or an antibody. Purity is measured by methods appropriate for the compound (e.g. chromatographic methods, polyacrylamide gel electrophoresis, HPLC analysis, and the like). Isolated antibodies as described herein are specifically purified from cell culture or provided as substantially pure proteins.

The term “therapeutically effective amount”, used herein interchangeably with any of the terms “effective amount” or “sufficient amount” of a compound, e.g. an antibody as described herein, is a quantity or activity sufficient to, when administered to the subject effect beneficial or desired results, including clinical results, and, as such, an effective amount or synonym thereof depends upon the context in which it is being applied.

An effective amount is intended to mean that amount of a compound that is sufficient to treat, prevent or inhibit such diseases or disorder. In the context of disease, therapeutically effective amounts of the antibody as described herein are specifically used to treat, modulate, attenuate, reverse, or affect a disease or condition that benefits from an inhibition of S. aureus or S. aureus pathogenesis.

The amount of the compound that will correspond to such an effective amount will vary depending on various factors, such as the given drug or compound, the pharmaceutical formulation, the route of administration, the type of disease or disorder, the identity of the subject or host being treated, and the like, but can nevertheless be routinely determined by one skilled in the art.

An antibody or combination preparation as described herein may be used prophylactically to inhibit onset of S. aureus infection, or therapeutically to treat S. aureus infection, particularly S. aureus infections such as MRSA that are known to be refractory or in the case of the specific subject, have proven refractory to treatment with other conventional antibiotic therapy.

A therapeutically effective amount of an antibody as described herein, such as provided to a human patient in need thereof, may specifically be in the range of 0.5-50 mg/kg, preferably 5-40 mg/kg, even more preferred up to 20 mg/kg, up to 10 mg/kg, up to 5 mg/kg, though higher doses may be indicated e.g. for treating acute disease conditions. The combination preparation may contain the respective therapeutically effective amounts of each of the antibodies.

Moreover, a treatment or prevention regime of a subject with a therapeutically effective amount of an antibody as described herein may consist of a single administration, or alternatively comprise a series of applications. For example, the antibody may be administered at least once a year, at least once a half-year or at least once a month. However, in another embodiment, the antibody may be administered to the subject from about one time per week to about a daily administration for a given treatment. The length of the treatment period depends on a variety of factors, such as the severity of the disease, either acute or chronic disease, the age of the patient, the concentration and the activity of the antibody format. It will also be appreciated that the effective dosage used for the treatment or prophylaxis may increase or decrease over the course of a particular treatment or prophylaxis regime. Changes in dosage may result and become apparent by standard diagnostic assays known in the art. In some instances, chronic administration may be required.

The invention specifically provides pharmaceutical compositions which comprise an antibody or the antibody combination as described herein and a pharmaceutically acceptable carrier or excipient. These pharmaceutical compositions can be administered in accordance with the present invention as a bolus injection or infusion or by continuous infusion. Pharmaceutical carriers suitable for facilitating such means of administration are well known in the art.

Pharmaceutically acceptable carriers generally include any and all suitable solvents, dispersion media, coatings, antibacterial and antifungal agents, isotonic and absorption delaying agents, and the like that are physiologically compatible with an antibody or related composition or combination provided by the invention. Further examples of pharmaceutically acceptable carriers include sterile water, saline, phosphate buffered saline, dextrose, glycerol, ethanol, and the like, as well as combinations of any thereof.

In one such aspect, an antibody can be combined with one or more carriers appropriate a desired route of administration, antibodies may be, e.g. admixed with any of lactose, sucrose, starch, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, polyvinyl alcohol, and optionally further tableted or encapsulated for conventional administration. Alternatively, an antibody may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other carriers, adjuvants, and modes of administration are well known in the pharmaceutical arts. A carrier may include a controlled release material or time delay material, such as glyceryl monostearate or glyceryl distearate alone or with a wax, or other materials well known in the art.

Additional pharmaceutically acceptable carriers are known in the art and described in, e.g. REMINGTON'S PHARMACEUTICAL SCIENCES. Liquid formulations can be solutions, emulsions or suspensions and can include excipients such as suspending agents, solubilizers, surfactants, preservatives, and chelating agents.

Pharmaceutical compositions are contemplated wherein an antibody as described herein and one or more therapeutically active agents are formulated. Stable formulations of an antibody are prepared for storage by mixing said antibody having the desired degree of purity with optional pharmaceutically acceptable carriers, excipients or stabilizers, in the form of lyophilized formulations or aqueous solutions. The formulations to be used for in vivo administration are specifically sterile, preferably in the form of a sterile aqueous solution. This is readily accomplished by filtration through sterile filtration membranes or other methods. The antibody and other therapeutically active agents disclosed herein may also be formulated as immunoliposomes, and/or entrapped in microcapsules.

Administration of the pharmaceutical composition comprising an antibody as described herein, may be done in a variety of ways, including orally, subcutaneously, intravenously, intranasally, intraotically, transdermally, mucosal, topically, e.g., gels, salves, lotions, creams, etc., intraperitoneally, intramuscularly, intrapulmonary, e.g. employing inhalable technology or pulmonary delivery systems, vaginally, parenterally, rectally, or intraocularly.

Examplary formulations as used for parenteral administration include those suitable for subcutaneous, intramuscular or intravenous injection as, for example, a sterile solution, emulsion or suspension.

In one embodiment, an antibody as described herein is the only therapeutically active agents administered to a subject, e.g. as a disease modifying or preventing monotherapy.

In another embodiment, an antibody as described herein is combined with further antibodies in a cocktail, e.g. combined in a mixture or kit of parts, to target S. aureus, such that the cocktail contains more therapeutically active agents administered to a subject, e.g. as a disease modifying or preventing combination therapy.

Alternatively, an antibody as described herein is administered in combination with one or more other therapeutic or prophylactic agents, including but not limited to standard treatment, e.g. antibiotics, steroid and non-steroid inhibitors of inflammation, and/or other antibody based therapy, e.g. employing anti-bacterial or anti-inflammatory agents.

A combination therapy is particularly employing a standard regimen, e.g. as used for treating MRSA infection. This may include antibiotics, e.g. tygecycline, linezolide, methicillin and/or vancomycin.

In a combination therapy, the antibody as described herein may be administered as a mixture, or concomitantly with one or more other therapeutic regimens, e.g. either before, simultaneously or after concomitant therapy.

Another aspect of the present invention provides a kit for therapeutic use, comprising the antibody as described herein and further active substances in different containers. The kit may include, in addition to the one or more antibodies, various other therapeutic agents and auxiliary agents and devices to prepare pharmaceutical formulations ready for use. A kit may also include instructions for use in a therapeutic method. Such instructions can be, for example, provided on a device included in the kit. In another specific embodiment, the kit includes an antibody in the lyophilized form, in combination with pharmaceutically acceptable carrier(s) that can be mixed before use to reconstitute the lyophilisate and to produce an injectable solution for near term administration.

The biological properties of the antibodies or the combination preparation as described herein or the respective pharmaceutical preparations may be characterized ex vivo in cell, tissue, and whole organism experiments. As is known in the art, drugs are often tested in vivo in animals, including but not limited to mice, rats, rabbits, dogs, cats, pigs, and monkeys, in order to measure a drug's efficacy for treatment against a disease or disease model, or to measure a drug's pharmacokinetics, pharmacodynamics, toxicity, and other properties. The animals may be referred to as disease models. Therapeutics are often tested in mice, including but not limited to nude mice, SCID mice, xenograft mice, and transgenic mice (including knockins and knockouts). Such experimentation may provide meaningful data for determination of the potential of the antibody to be used as a therapeutic or as a prophylactic with the appropriate half-life, effector function, (cross-) neutralizing activity and/or immune response upon active or passive immunotherapy. Any organism, preferably mammals, may be used for testing. For example, because of their genetic similarity to humans, primates, monkeys can be suitable therapeutic models, and thus may be used to test the efficacy, toxicity, pharmacokinetics, pharmacodynamics, half-life, or other property of the subject agent or composition. Tests in humans are ultimately required for approval as drugs, and thus of course these experiments are contemplated. Thus, the antibodies or the combination preparation as described herein and respective pharmaceutical compositions of the present invention may be tested in humans to determine their therapeutic or prophylactic efficacy, toxicity, immunogenicity, pharmacokinetics, and/or other clinical properties.

The invention also provides the subject antibody of the invention for diagnostic purposes, e.g. for use in methods of detecting and quantitatively determining the concentration of an IGBP as immunoreagent or target in a biological fluid sample.

The invention also provides methods for detecting the level of IGBP or S. aureus infection in a biological sample, such as a body fluid, comprising the step of contacting the sample with an antibody of the invention. The antibody of the invention may be employed in any known assay method, such as competitive binding (in competition with IgG-Fc binding) assays, direct and indirect sandwich assays, immunoprecipitation assays and enzyme-linked immunosorbent assays (ELISA).

A body fluid as used according to the present invention includes biological samples of a subject, such as tissue extract, urine, blood, serum, stool and phlegm.

In one embodiment the method comprises contacting a solid support with an excess of a certain type of antibody fragment which specifically forms a complex with a target, such as at least one of the IGBP domains targeted by the antibody of the invention, conditions permitting the antibody to attach to the surface of the solid support. The resulting solid support to which the antibody is attached is then contacted with a biological fluid sample so that the target in the biological fluid binds to the antibody and forms a target-antibody complex. The complex can be labeled with a detectable marker. Alternatively, either the target or the antibody can be labeled before the formation the complex. For example, a detectable marker (label) can be conjugated to the antibody. The complex then can be detected and quantitatively determined thereby detecting and quantitatively determining the concentration of the target in the biological fluid sample.

For particular applications an antibody of the invention is conjugated to a label or reporter molecule, selected from the group consisting of organic molecules, enzyme labels, radioactive labels, colored labels, fluorescent labels, chromogenic labels, luminescent labels, haptens, digoxigenin, biotin, metal complexes, metals, colloidal gold and mixtures thereof. Antibodies conjugated to labels or reporter molecules may be used, for instance, in assay systems or diagnostic methods, e.g. to diagnose S. aureus infection or disease conditions associated therewith.

The antibody of the invention may be conjugated to other molecules which allow the simple detection of said conjugate in, for instance, binding assays (e.g. ELISA) and binding studies.

The subject matter of the following definitions is considered embodiments of the present invention:

1. A monoclonal antibody that counteracts or neutralizes Staphylococcus aureus by specifically binding to wild-type immunoglobulin-binding proteins (IGBP) of S. aureus comprising a cross-specific CDR binding site recognizing at least three of the

IGBP domains selected from the group consisting of Protein A (SpA) domains and immunoglobulin-binding protein (Sbi) domains SpA-A, SpA-B, SpA-C, SpA-D, SpA-E, Sbi-I, and Sbi-II, wherein the antibody has an affinity to bind SpA-E with a K_{D} of less than 5×10⁻⁹M, as determined by a standard optical interferometry method for a F(ab)2 fragment.

2. The antibody of definition 1, which recognizes at least three of the IGBP domains, preferably at least four, five, or six of the IGBP domains.

3. The antibody of definition 1 or 2, which recognizes at least three of the IGBP domains each with a K_{D} of less than 5×10⁻⁹M, preferably at least four or five of the IGBP each with a K_{D} of less than 5×10⁻⁹M.

4. The antibody of any of definitions 1 to 3, which recognizes the wild-type SpA with at least substantially the same affinity or with substantially higher affinity as compared to the mutant SpA that lacks binding to IgG Fc or VH3, or as compared to the mutant SpA_{KK} or SpA_{KKAA}, preferably wherein the wild-type SpA is any of the SpA-domains comprising the sequence identified by SEQ ID 145 and optionally further comprising the sequence identified by SEQ ID 146, preferably as determined by comparing the affinity to bind the wild-type SpA-D comprising the amino acid sequence SEQ ID 138 and the mutant SpA-D_{KKAA} comprising the amino acid sequence SEQ ID 143.

5. The antibody of any of definitions 1 to 4, which recognizes both, SpA and Sbi.

6. The antibody of any of definitions 1 to 5, which competes with SpA and optionally Sbi binding to IgG-Fc.

7. The antibody of any of definitions 1 to 6, which is neutralizing Staphylococcus aureus by enhanced opsonophagocytosis and killing by phagocytic cells. 8. The antibody of any of definitions 1 to 6, which is a full-length monoclonal antibody, an antibody fragment thereof comprising at least one antibody domain incorporating the binding site, or a fusion protein comprising at least one antibody domain incorporating the binding site, specifically wherein the antibody is a non-naturally occurring antibody which comprises a randomized or artificial amino acid sequence.

9. The antibody of any of definitions 1 to 8, which comprises at least an antibody heavy chain variable region (VH), which is characterized by any of the CDR1 to CDR3 sequences as listed in Table 1, and optionally an antibody light chain region (VL), which is characterized by any of the CDR4 to CDR6 sequences as listed in Table 1, which CDR sequences are designated according to the numbering system of Kabat, or functionally active CDR variants of any of the foregoing.

10. The antibody of definitions 9, which
a) comprises a VH domain, which is characterized by any of the CDR1 to

CDR3 sequence combinations as listed in Table 1, and a VL domain, which is characterized by any of the CDR4 to CDR6 sequence combinations as listed in Table 1;
b) comprises the set of CDR sequences (CDR1-CDR6) of any of the antibodies as listed in Table 1;
c) is any of the antibodies as listed in Table 1; or
d) is a functionally active variant of a parent antibody that is characterized by the sequences of a)-c),
   preferably wherein
      i. the functionally active variant comprises at least one functionally active CDR variant of any of the CDR1-CDR6 of the parent antibody; and/or
      ii. the functionally active variant comprises at least one point mutation in the framework region of any of the VH and VL sequences;
   and further wherein
      iii. the functionally active variant has a specificity to bind the same epitope as the parent antibody; and/or
      iv. the functionally active variant is a human, humanized, chimeric or murine and/or affinity matured variant of the parent antibody.

11. The antibody of definitions 9 or 10, comprising a functionally active CDR variant of any of the CDR sequences as listed in Table 1, wherein the functionally active CDR variant comprises at least one of

a) 1, 2, or 3 point mutations in the parent CDR sequence; and/or

b) 1 or 2 point mutations in any of the four C-terminal or four N-terminal, or four centric amino acid positions of the parent CDR sequence; and/or

c) at least 60% sequence identity with the parent CDR sequence;

preferably wherein the functionally active CDR variant comprises 1 or 2 point mutations in any CDR sequence.

12. The antibody of any of definitions 9 to 11, which is selected from the group consisting of group members i) to vi), wherein

i)

A) the antibody comprises
a) a CDR1 consisting of the amino acid sequence of SEQ ID 13; and
b) a CDR2 consisting of the amino acid sequence of SEQ ID 14; and
c) a CDR3 consisting of the amino acid sequence of SEQ ID 15;
and optionally further comprises
d) a CDR4 consisting of the amino acid sequence of SEQ ID 73; and
e) a CDRS consisting of the amino acid sequence of SEQ ID 74; and
f) a CDR6 consisting of the amino acid sequence of SEQ ID 75;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 13;
b) the parent CDR2 consists of the amino acid sequence SEQ ID 14;
c) the parent CDR3 consists of the amino acid sequence SEQ ID 15;
d) the parent CDR4 consists of the amino acid sequence SEQ ID 73;
e) the parent CDRS consists of the amino acid sequence SEQ ID 74;
f) the parent CDR6 consists of the amino acid sequence SEQ ID 75;

ii)

A) the antibody comprises
a) a CDR1 consisting of the amino acid sequence of SEQ ID 31; and
b) a CDR2 consisting of the amino acid sequence of SEQ ID 32; and
c) a CDR3 consisting of the amino acid sequence of SEQ ID 33;

and optionally further comprises
d) a CDR4 consisting of the amino acid sequence of SEQ ID 91; and
e) a CDR5 consisting of the amino acid sequence of SEQ ID 92; and
f) a CDR6 consisting of the amino acid sequence of SEQ ID 93;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 31;
b) the parent CDR2 consists of the amino acid sequence SEQ ID 32;
c) the parent CDR3 consists of the amino acid sequence SEQ ID 33;
d) the parent CDR4 consists of the amino acid sequence SEQ ID 91;
e) the parent CDRS consists of the amino acid sequence SEQ ID 92;
f) the parent CDR6 consists of the amino acid sequence SEQ ID 93;

iii)

A) the antibody comprises
a) a CDR1 consisting of the amino acid sequence of SEQ ID 40; and
b) a CDR2 consisting of the amino acid sequence of SEQ ID 41; and
c) a CDR3 consisting of the amino acid sequence of SEQ ID 42;
and optionally further comprises
d) a CDR4 consisting of the amino acid sequence of SEQ ID 100; and
e) a CDR5 consisting of the amino acid sequence of SEQ ID 101; and
f) a CDR6 consisting of the amino acid sequence of SEQ ID 102;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 40;
b) the parent CDR2 consists of the amino acid sequence SEQ ID 41;
c) the parent CDR3 consists of the amino acid sequence SEQ ID 42;
d) the parent CDR4 consists of the amino acid sequence SEQ ID 100;
e) the parent CDRS consists of the amino acid sequence SEQ ID 101;
f) the parent CDR6 consists of the amino acid sequence SEQ ID 102;

iv)

A) the antibody comprises
a) a CDR1 consisting of the amino acid sequence of SEQ ID 43; and
b) a CDR2 consisting of the amino acid sequence of SEQ ID 44; and
c) a CDR3 consisting of the amino acid sequence of SEQ ID 45;

and optionally further comprises
d) a CDR4 consisting of the amino acid sequence of SEQ ID 103; and
e) a CDR5 consisting of the amino acid sequence of SEQ ID 104; and
f) a CDR6 consisting of the amino acid sequence of SEQ ID 105;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 43;
b) the parent CDR2 consists of the amino acid sequence SEQ ID 44;
c) the parent CDR3 consists of the amino acid sequence SEQ ID 45;
d) the parent CDR4 consists of the amino acid sequence SEQ ID 103;
e) the parent CDR5 consists of the amino acid sequence SEQ ID 104;
f) the parent CDR6 consists of the amino acid sequence SEQ ID 105;

v)

A) the antibody comprises
a) a CDR1 consisting of the amino acid sequence of SEQ ID 46; and
b) a CDR2 consisting of the amino acid sequence of SEQ ID 47; and
c) a CDR3 consisting of the amino acid sequence of SEQ ID 48;

and optionally further comprises
d) a CDR4 consisting of the amino acid sequence of SEQ ID 106; and
e) a CDR5 consisting of the amino acid sequence of SEQ ID 107; and
f) a CDR6 consisting of the amino acid sequence of SEQ ID 108;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 46;
b) the parent CDR2 consists of the amino acid sequence SEQ ID 47;
c) the parent CDR3 consists of the amino acid sequence SEQ ID 48;
d) the parent CDR4 consists of the amino acid sequence SEQ ID 106;
e) the parent CDR5 consists of the amino acid sequence SEQ ID 107;
f) the parent CDR6 consists of the amino acid sequence SEQ ID 108;

and

vi)

A) the antibody comprises
a) a CDR1 consisting of the amino acid sequence of SEQ ID 58; and
b) a CDR2 consisting of the amino acid sequence of SEQ ID 59; and
c) a CDR3 consisting of the amino acid sequence of SEQ ID 60;

and optionally further comprises
d) a CDR4 consisting of the amino acid sequence of SEQ ID 118; and
e) a CDRS consisting of the amino acid sequence of SEQ ID 119; and
f) a CDR6 consisting of the amino acid sequence of SEQ ID 120;

or

B) the antibody is an antibody of A, wherein at least one of the CDR is a functionally active CDR variant of a parent CDR, comprising at least one point mutation in the parent CDR and at least 60% sequence identity with the parent CDR, wherein
a) the parent CDR1 consists of the amino acid sequence SEQ ID 58;
b) the parent CDR2 consists of the amino acid sequence SEQ ID 59;
c) the parent CDR3 consists of the amino acid sequence SEQ ID 60;
d) the parent CDR4 consists of the amino acid sequence SEQ ID 118;
e) the parent CDRS consists of the amino acid sequence SEQ ID 119;
f) the parent CDR6 consists of the amino acid sequence SEQ ID 120.

13. The antibody of any of definitions 1 to 12, for use in treating a subject at risk of or suffering from S. aureus infection or colonization comprising administering to the subject an effective amount of the antibody to limit the infection in the subject or to ameliorate a disease condition resulting from said infection or to inhibit S. aureus disease pathogenesis, such as pneumonia, sepsis, bacteremia, wound infection, abscesses, surgical site infection, endothalmitis, furunculosis, carbunculosis, endocarditis, peritonitis, osteomyelitis or joint infection.

14. A pharmaceutical preparation comprising the antibody of any of definitions 1 to 12, preferably comprising a parenteral or mucosal formulation, optionally containing a pharmaceutically acceptable carrier or excipient.

15. The antibody according to any of definitions 1 to 12, for diagnostic use to detect any S. aureus infections, including high toxin producing MRSA infections, such as necrotizing pneumonia, and toxin production in furunculosis and carbunculosis.

16. A diagnostic preparation of the antibody according to any of definitions 1 to 12, optionally containing the antibody with a label and/or a further diagnostic reagent with a label.

17. An isolated nucleic acid encoding an antibody according to any of definitions 1 to 12.

18. A recombinant expression cassette or a plasmid comprising a coding sequence to express a light chain and/or heavy chain of an antibody according to any of definitions 1 to 12.

19. A host cell comprising the expression cassette or the plasmid of definition 18.

20. A method of producing an antibody according to any of definitions 1 to 12, wherein a host cell according to definition 19 is cultivated or maintained under conditions to produce said antibody.

21. A method of producing a functionally active variant antibody of a parent antibody which parent antibody is any of the antibodies characterized by the CDR sequences as listed in Table 1, which method comprises engineering at least one point mutation in any of the framework regions (FR) or constant domains, or any of the CDR sequences of the parent antibody to obtain a variant antibody, and determining the functional activity of the variant antibody by its affinity to bind SpA and/or Sbi with a K_{D} of less than 10⁻⁸M, preferably less than 9×10⁻⁹M, wherein upon determining the functional activity, the functionally active variant antibody is selected for production by a recombinant production method.

The foregoing description will be more fully understood with reference to the following examples. Such examples are, however, merely representative of methods of practicing one or more embodiments of the present invention and should not be read as limiting the scope of invention.

### EXAMPLES

### Example 1

### Generation of Protein Baits Representing the IgG Binding Domains of SpA and Sbi

The two IgG binding proteins of S. aureus, SpA and Sbi, are multi-domain proteins and contain 5 and 2 IgG binding domains, respectively (FIG. 3). The SpA domains A-E share 63-91% amino acid sequence identity, the two Sbi domains are much less homologues to each other or to the SpA domains (27-32%) (FIG. 4A,B). These domains are highly conserved in the different S. aureus strains, SpA displays sequence variations outside of the five IgG binding domains in the C-terminus (SEQ IDs: 121-134). To support the selection and characterization of monoclonal antibodies binding to S. aureus IgG binding proteins, the five IgG binding domains of Protein A (SpA) and the two domains of Sbi were recombinantly generated in E. coli Tuner DE3 cells. DNA sequences of the 7 domains were derived from the published genome sequences of Staphylococcus aureus strain USA300_TCH1516 (SEQ IDs 135-141) and were optimized for E. coli expression. C-terminal Gly-Gly-Cys amino acids were added to each genes to allow site-specific labeling with biotin. The expression plasmids cloned in pET16b were transformed in E. coli Tuner DE3, the cells were grown at 37° C. until an OD₆₀₀ of 0.6-1.0 was reached, and induced with 0.5 mM IPTG at 37° C. for 4 h. The cell pellet was resuspended in 50 mM Tris, pH 7.5 plus 150 mM NaCl and 0.05% Tween 20 (buffer A) containing benzonase, rLysosyme (Novagen) and protease inhibitor (Roche) and the cells were disrupted by sonication. The cleared lysate was loaded onto an IgG-Sepharose FF affinity matrix (5 ml packed in a Tricorn 10/50 column, GE Healthcare) equilibrated with buffer A. The column was washed with buffer A followed by 5 mM sodium acetate pH 5.0, and the proteins eluted with 0.5 M sodium acetate pH 3.4. The fractions containing the target protein were neutralized, reduced [with 10 mM dithiotreitol (DTT) for ˜2 h at 4° C.] and further purified using a Superdex 75 (16/60, GE Healthcare) gel filtration column equilibrated in 50 mM sodium phosphate pH 7.2 plus 150 mM NaClThe proteins were assayed for purity (by SDS-PAGE) and monomeric state (by size exclusion), as well as for functionality, i.e. binding to the IgG1 (via Fc and VH3 domains), as described in Example 2. All proteins were labeled on C-terminal cysteine with the sulfhydryl reactive reagent EZ-Link Biotin-BMCC (Thermo Scientific). For the non-labeled proteins the free Cys was blocked with iodoacetamide.

### Example 2

### Discovery of Human Monoclonal Antibodies Using Yeast Expressed Human IgG Libraries and SpA IgG Binding Domains with Wild Type Sequences

SpA and Sbi binding monoclonal antibodies were selected from yeast-based antibody presentation libraries developed according to WO2009/036379A2, WO2010105256 and WO2012009568.

A library of yeast cells engineered to express full length human IgG1 antibodies with an approx. 10⁹ diversity were incubated with different concentrations of native full length SpA (purified from S. aureus NCTC 8325, purchased from Sigma, cat #P6031), or recombinant Spa-D domain, the former biotinylated using the amino reactive reagent Sulfo-NHS-LC (Thermo Scientific) and the latter, as described above. To avoid non-specific binding via Fab domains, all VH3 germline sequences were excluded from the libraries used for the selection. Yeast cells expressing antibodies with the capacity of binding to these baits were isolated by magnetic bead selection and fluorescence-activated cell sorting (FACS) employing streptavidin secondary reagents in several successive (up to five) selection rounds. Antibodies were then produced by the selected yeast clones and purified by Protein A affinity chromatography. Binding of individual, soluble mAbs to the target proteins was confirmed by interferometry measurements using a ForteBio Octet Red instrument [Pall Life Sciences]; the biotinylated antigen was immobilized on streptavidin sensors to give a sensor loading of ˜2 nm. The association and dissociation of the antibody F(ab′)₂ or F(ab) fragments (50-100 and 100-200 nM, respectively), in solution (PBS, pH 7.2 plus 1% BSA), were measured at 30° C. for 3-10 min for the association phase and 3-30 min for the dissociation phase. The dissociation constants (K_{D} values) were calculated based on the kinetic parameters (kon and koff) determined by fitting simultaneously the association and dissociation phases to a 1:1 binding model using Octet Data Analysis Software version 7.

In the first round of selection, 46 mAbs with unique sequences were discovered with the two baits. Non-Fc fragment dependent binding of these antibodies were proven already in the affinity measurement, since Fab fragments were used. Since the library was pre-selected not to contain VH3 germline, non-specific interactions of Fabs were also excluded.

Typically Fab and F(ab′)₂ fragments were prepared by papain and pepsin digestion, respectively, of full length IgGs produced in yeast and purified by protein A chromatography. Fab fragments were purified by Capture Select LC-kappa, while F(ab′)₂ fragments were purified by CaptureSelect IgG-CH1 affinity chromatography (Life Technologies). In selected cases, mAbs were produced in CHO cells as full IgGs, purified by mAbSelect SuRe (GE Healthcare) affinity chromatography, and the F(ab)2 fragments generated and purified as described for the yeast antibodies.

The 46 antibodies had a broad range of binding activities to the five different IgG-binding domains of SpA and showed different patterns measured by ForteBio Octet using biotinylated recombinant domains immobilized on Streptavidin coated sensor tips (5 μg/rd) and F(ab′)₂ domains of IgGs added (in PBS, pH 7.2, with 1% BSA) at 100 nM concentration. In this measurement (FIG. 5), the binding strength after 10 min (at 30° C.) is expressed as response unit (RU). Three mAbs - 10092, 10322 and 10323—bound to all five domains, while others to only one domain, or 2 to 4 domains. For example 12 mAbs were specific for SpA domain E (e.g. 10289, 10314 and 10319), one for domain A (10279) or domain B (10296) or domain C (10310). 18 antibodies bound to the three N-terminal domains E, D and A. Interestingly, the multi-domain binding anti-SpA antibodies also recognized the Sbi domain I.

12 of the 46 antibodies (indicated in bold in FIG. 5) were selected for affinity maturation in successive selection rounds, resulting in 77 offsprings in total. Increased antibody binding to SpA with improved affinities was observed with offsprings in most lineages (example shown in FIG. 6A,B; here, the association of 100 nM Fab or F(ab′)2 fragment to biotinylated native SpA or recombinant SpA or Sbi domains immobilized on streptavidin sensors, in PBS, pH 7.2 plus 1% BSA at 30° C., was measured for 3 min (to give binding responses), while the dissociation, in the same buffer, was monitored for 3 min).

Affinity measurements were performed by optical interferometry using a ForteBio Octet Red instrument [Pall Life Sciences] as described above. In general F(ab′)₂s derived from CHO expressed IgGs were more pure, i.e., showed less Fab contamination, as judged from non-reducing SDS-PAGE, and showed higher affinities (lower dissociation constants) than those derived from yeast IgGs. For comparison purposes, affinity values with F(ab′)2s from the two sources are shown in Table 1c.

For the measurement of selectivity of the anti-SpA mAbs towards wild-tye (WT) SpA compared to SpA KKAA, binding of the mAbs to SpA-D (SEQ ID 138) and SpA-D KKAA (SEQ ID 143) was determined using biotinylated antigens as described above. SpA-D KKAA was expressed recombinantly, purified and biotinylated as described for the wild-type domains, except an anion exchange chromatography step on Q Sepharose FF column (GE Healthcare) was used instead of the IgG Sepharose affinity chromatography; the column was equilibrated in 20 mM Tris, pH 8.0 and the protein eluted with a linear gradient of 0 - 1 M NaCl in the same buffer. The anti-SpA mAbs showed decreased binding to the KKAA variant, as opposed to 3F6, which has preference for the SpA-D KKAA (Table 1c).

It is known that recombinantly expressed antibodies are prone to a series of post-translational modifications, among which the pyroglutamate formation, particularly when glutamine is present at the N-terminus of the heavy chain, and cleavage of the C-terminal lysine residue, also from the heavy chain (Liu H, Ponniah G, Zhang H M, Nowak C, Neill A, Gonzalez-Lopez N, Patel R, Cheng G, Kita A Z, Andrien B. In vitro and in vivo modifications of recombinant and human IgG antibodies. MAbs. 2014; 6(5):1145-54). Therefore, for selected antibodies expressed in CHO cells, the N-terminus glutamine was mutated to glutamate, and the C-terminal lysine was removed, to avoid sample heterogeneity, giving Q1EΔK variants (e.g. SEQ ID153, 155, 157, 159). These mutations are not expected to affect mAb properties, i.e. antigen binding and Fc function (Liu H, Ponniah G, Zhang H M, Nowak C, Neill A, Gonzalez-Lopez N, Patel R, Cheng G, Kita A Z, Andrien B. In vitro and in vivo modifications of recombinant and human IgG antibodies. MAbs. 2014; 6(5):1145-54).

The presence of a non-immune SpA binding site in the Fc region of an anti-SpA mAbs could in principle interfere with Fc receptor binding by two mechanisms. Potential avid binding of the mAb via both the variable and constant regions would present the mAb on the bacterial surface in an orientation that could hinder engagement of the Fc gamma receptors. Secondly, even if the Fc would not be engaged in an interaction with surface bound protein A, soluble protein A (that can be shed from the bacteria surface) could bind/cross-link the antibodies presented on the bacterium and sterically prevent binding of the Fc gamma receptors. Both mechanisms could result in impaired Fc gamma receptor mediated phagocytosis. Thus, in the Fc region of the anti-SpA mAbs mutations were introduced that are known in the art to reduce/abolish SpA binding, i.e. His435Arg and Tyr436Phe (Stapleton N M, Andersen J T, Stemerding A M, Bjarnarson S P, Verheul R C, Gerritsen J, Zhao Y, Kleijer M, Sandlie I, de Haas M, Jonsdottir I, van der Schoot C E, Vidarsson G. Competition for FcRn-mediated transport gives rise to short half-life of human IgG3 and offers therapeutic potential. Nat Commun. 2011 Dec. 20; 2:599; WO2003063772 A2) These mutations are also present in the naturally occurring IgG3 isotype, which is known to be devoid of protein A binding. Moreover, these residues are found at the CH2-CH3 interface, remote from the Fc gamma receptor binding sites, and the mutations are not expected to interfere with the immune functions of the antibody (Deisenhofer, J. Crystallographic refinement and atomic models of a human Fc fragment and its complex with fragment B of Protein A from S. aureus at 2.9- and 2.8-A resolution, Biochemistry 20, 1981, 2361; Shields R L, Namenuk A K, Hong K, Meng Y G, Rae J, Briggs J, Xie D, Lai J, Stadlen A, Li B, Fox J A, Presta L G. High resolution mapping of the binding site on human IgG1 for Fc gamma RI, Fc gamma RII, Fc gamma RIII, and FcRn and design of IgG1 variants with improved binding to the Fc gamma R. J Biol Chem. 2001 Mar. 2; 276(9):6591-604; Wines B D, Powell M S, Parren P W, Barnes N, Hogarth P M. The IgG Fc contains distinct Fc receptor (FcR) binding sites: the leukocyte receptors Fc gamma RI and Fc gamma RIla bind to a region in the Fc distinct from that recognized by neonatal FcR and protein A. J Immunol. 2000 May 15; 164(10):5313-8; Bruhns P, lannascoli B, England P, Mancardi D A, Fernandez N, Jorieux S, Daëron M. Specificity and affinity of human Fcgamma receptors and their polymorphic variants for human IgG subclasses. Blood. 2009 Apr. 16; 113(16):3716-25). For the 10901 mAb the His435Arg single mutant (R, heavy chain SEQ ID 162, light chain SEQ ID 163), and the His435Arg, Tyr436Phe double mutant (RF heavy chain SEQ ID 161,light chain SEQ ID 163) was produced in CHO cells, and purified by MabSelect SuRe affinity chromatography (GE Healthcare) and their activity was tested in OPK assays as described in Example 5. In addition, a triple ‘QRF’ Fc mutant (Lys274Gln, His435Arg, Tyr436Phe) was produced for 10901 (heavy chain SEQ ID 160,light chain SEQ ID 163), the Lys274Gln mutation preventing Fc binding to the S. aureus surface antigen SSL10 (Patel D, Wines B D, Langley R J, Fraser J D. Specificity of staphylococcal superantigen-like protein 10 toward the human IgG1 Fc domain. J Immunol. 2010 Jun. 1; 184(11):6283-92), which triple mutant was reported to enhance OPK of an anti-SpA antibody (WO/2013/096948), to compare it to the single ‘R’ and double ‘RF’ mutants (Example 5).

### Example 3

### Specific Binding of anti-SpA mAbs to Live S. aureus Cells

Binding of anti-SpA antibodies to live S. aureus bacteria was tested with TCH1516 (USA300 MRSA strain) by flow cytometry. To exclude any potential interference with Fc-binding and to prove that binding is mediated by the CDR region, antibodies were tested either as Fab or F(ab′)₂ fragments. Notably, none of the tested antibodies (including the negative control antibody) belonged to the VH3 family that is known to bind SpA via the Fab region (Sasso et. al., 1991).

Bacteria were grown to mid-logarithmic phase in RPMI (Life Technologies) supplemented with 1% casamino acids (Amresco). The culture was washed with Hank's balanced salt solution (HBSS, Life Technologies) supplemented with 0.5% bovine serum albumin (BSA, PAA) and 0.01% sodium azide. 1 ×10⁶ bacteria were incubated with 10, 2 or 0.4 nM of anti-SpA F(ab′)₂ fragments for 45 min on ice. After washing with HBSS supplemented with 0.01% sodium azide to remove unbound antibodies, bacteria were stained with an Alexa 488 labeled goat anti-human-IgG F(ab′)₂ fragment as secondary reagent (Jackson ImmunoResearch) at 500×. For identification of bacteria in the flow based assay, bacteria were further stained with 5 pM SYTO 62 (Life Technologies) for 10 min at room temperature (RT) prior to measurement using an iCyt eclipse flow cytometer. Data were analyzed with FCS Express software (Version 4, Flow Research Edition).

S. aureus surface binding was detected with all 123 anti-SpA F(ab′)₂ fragments generated during this discovery work. The binding intensity ranged from low to very high with the individual antibodies and was dose dependent in the 0.4-100 nM range (examples shown in FIG. 7 using 10 nM concentration). Importantly no binding was detected with the negative control F(ab′)₂ specific for an irrelevant antigen or with anti-SpA F(ab′)₂ to an isogenic mutant lacking the genes for spa and sbi (examples shown in FIG. 8).

Increase in binding and affinity was observed among offsprings relative to parents measured by fortéBio, which was typically translated to improved surface binding to S. aureus (examples shown in FIG. 9).

### Example 4

### Inhibition of Fc-Mediated Binding of Human IgG to SpA by Anti-SpA Antibodies

The inhibition of IgG binding to SpA is likely to be an important mode of action of anti-SpA mAbs in order to counteract immune evasion mechanisms by the IgG binding proteins of S. aureus. This was tested in two different assay systems.

In the fortéBio based binding assay, biotinylated native full length SpA was immobilized on Streptavidin coated sensors (final loading of ˜2 nm), followed by the association (10 min) of the anti-SpA F(ab′)₂ (10 μg/ml) (association 1), and the subsequent association of the human non-VH3 IgG (10 μg/ml) (association 2) onto the sensors. All experiments were performed in PBS, pH 7.2 plus 1% BSA, at 30° C. Each association was followed by a 5 min dissociation step in the same buffer. The % binding response observed during the second association was then calculated (based on 100% binding in the absence of anti-SpA F(ab′)₂ fragments (examples shown in FIG. 10).The 10272, and 10893 showed low competition, while 10886, 10901 and 10919 displayed potent inhibition of Fc mediated IgG binding.

The interference of anti-SpA antibodies with human IgG binding to S. aureus cells was tested in a flow cytometry based surface staining assay. S. aureus (TCH1516 wt strain) was pre-incubated with anti-SpA F(ab′)₂ fragments at a fixed concentration (100 nM), and after a washing step to remove unbound F(ab′)₂ fragments, bacteria were incubated with Alexa 488 fluorochrome labeled human IgG mAb not binding to S. aureus via its CDR. A broad range of IgG binding inhibition activities of anti-SpA F(ab′)₂ fragments was detected, ranging from no effect to significant inhibition (examples shown in FIG. 11). For example, in the experimental setting using 160 and 32 ng/ml concentration of labeled human IgG (1.07 and 0.21 nM, respectively), the 10279 and 10313 F(ab′)₂ fragments were ineffective, while 10092 and 10298 significantly reduced IgG binding to S. aureus (up to 70%).

### Example 5

### Binding of Anti-SpA full IgGs to S. aureus in Presence of Human Serum IgGs

Human serum contains 10-14 mg/ml IgG that are able to bind to SpA via their Fc region. The question was addressed whether anti-SpA mAbs are able to bind to SpA on the bacterial surface to mediate potential opsonophagocytic effect when bacteria are surrounded byserum and SpA is occupied by serum IgG attached via their Fc. This was tested in flow cytometry based assay with fluorochrome labelled anti-SpA mAbs binding to S. aureus in presence of human serum. For this assay, S. aureus TCH1516 was grown to mid-logarithmic phase in RPMI supplemented with 1% casamino acids. The culture was washed with HBSS supplemented with 0.5% BSA and 0.01% sodium azide. 1×10⁶ bacteria were incubated with a 346× diluted human serum (PAA) resulting in approximately 35pg/ml total IgG for 30 min on ice, to allow Fc-mediated binding of serum IgGs to the bacterial surface. Following this pre-incubation, bacteria were stained with fluorochrome labeled anti-SpA full IgGs at 1-10 nM concentration range (0.15-1.5 μμg/ml for 45 min on ice in the dark. Samples were washed with HBSS supplemented with 0.01% sodium azide and stained with 5 μM SYTO 62 (Life Technologies) for 10 min at RT and then measured using an iCyt eclipse flow cytometer. Data were analyzed with FCS Express software (Version 4, Flow Research edition). The concentrations used in these experiments correspond to a ratio of specific antibody vs. total serum IgG of 1:200, 1:80 and 1:20. These ratios in humans can be achieved by administering mAbs at doses between 2.5-25 mg/kg body weight.

In absence of competing serum antibodies, all anti-SpA mAbs but also the negative control antibody showed binding to live to S. aureus (not possible to distinguish between specific antibodies and Fc binding). However, binding of the labeled negative control antibody was observed to decrease much faster with increasing total IgG/labeled test IgG ratios compared to SpA-specific IgGs (examples shown in FIG. 12). These data confirm that CDR mediated antigen binding of SpA-specific mAbs do occur even if Fc binding sites are occupied with non-specific immunoglobulins. This is likely to be the consequence of higher affinity and avidity of the anti-SpA CDRs (faster association and slower disassociation) to SpA compared to non-SpA specific IgGs that associate only via Fc mediated binding with a K_{D} of approximately 10⁻⁷ M to individual domains and 10⁻⁸ M to full length SpA with all five domains. The highest affinity SpA-specific mAbs bind with at least 1 log higher affinity.

To support this conclusion, the surface binding intensity of anti-SpA antibodies before and after affinity maturation was compared. In every lineage affinity maturation resulted in improved binding of SpA-specific offspring IgGs relative to their parents (examples shown in FIG. 13).

### Example 6

### Measuring Opsonophagocytic Killing (OPK) Activity of Anti-SpA Monoclonal Antibodies

Enhancing opsonophagocytic uptake and phagocytic killing of pathogens is a common mode of action of antibodies raised against bacterial pathogens. Against Gram positive organisms it is the main antibacterial mechanism since complement mediated killing is not possible due to the Gram positive cell wall. Therefore it is important to assess whether a yeast platform derived, mammalian-cell expressed anti-SpA antibody can contribute to S. aureus uptake and killing in presence of professional phagocytes. Survival of S. aureus was determined in an in vitro OPK assay using freshly isolated human polymorphonuclear cells (PMNs). S. aureus TCH1516 and the isogenic ΔspAΔsbi mutant (used as specificity control) were grown to mid-logarithmic phase in RPMI supplemented with 1% casamino acids. The culture was then washed with assay buffer (50 g/L human Albumin (Albiomin, Biotest) in RPMI supplemented with 2 mM L-Glutamine and 2 mg/mL Sodium bicarbonate) and diluted to 8.6×10⁴ CFU/ml. Bacteria (20 μl) were pre-opsonized with test and control IgGs at 100 μg/ml concentration for 15 min with agitation at 37° C. Human PMNs, purified from human whole blood by a 2-step Percoll gradient centrifugation (Rouha et al., 2015) were diluted to 1.7×10⁷ cells/ml and seeded in 25 pl volumes into half-area flat bottom 96-well plates. Cells were allowed to sediment for 15 min (37° C., 5% CO₂) before addition of pre-opsonized bacteria to the cell layer in a volume of 75 pl. Bacteria and PMNs were compacted (synchronized) by centrifugation for 8 min at 525×g After 1 hour incubation at 37° C. and 5% CO₂, the reaction was stopped by putting the plate on ice. Content of the wells was re-suspended vigorously in the presence of 1% Saponin to lyse phagocytic cells. After an additional 10 min incubation step on ice to ensure complete cell lysis, samples were serially diluted in water and 100 μl were plated in duplicates on Tryptic-Soy-Agar (TSA) plates and incubated o/n at 37° C. for CFU enumeration on the next day.

It was observed CHO expressed IgG1 mAbs (selected against wt protein A) in these assays, a significant reduction in S. aureus survival was observed (see example for mAb 10901-SEQ IDs 159,163 in FIG. 14). This effect was specific to SpA since killing of the AspAAsbi mutant isogenic S. aureus strain was not different from controls (opsonized with non-specific IgG1) Noteably, all Fc mutations introduced into 10901 to reduce/abolish Fc-mediated SpA binding (10901_QRF - SEQ IDs 160,163; 10901_RF-SEQ IDs 161,163; and 10901_R - SEQ IDs 162,163) further increased the opsonophagocytic potency of the antibody, increasing killing from ˜40% to ˜80%.

### Example 7

### Efficacy testing of Anti-SpA IgGs in Murine Bacteremia/Sepsis Model with Intravenous S. aureus Challenge

The pathogen specific antibodies were tested in in vivo models to determine if they contribute to protection from infectious diseases. SpA-specific antibodies may have several modes of action, such as inhibition of SpA's virulence factor functions (e.g. IgG-Fc binding) and enhancement of phagocytic uptake and killing of S. aureus by targeting surface expressed SpA.

Several SpA-specific human IgG1 antibodies expressed in CHO cells were tested in a lethal S. aureus challenge model induced by intravenous injection of bacteria. The S. aureus strain TCH1516 (USA300 CA-MRSA; BAA-1717™, ATCC) was grown to mid-log phase (OD₆₀₀ of 0.5) in tryptic soy broth and diluted for intravenous injection (100 μl with 2×10⁷ cfu challenge dose per animal). In all experiments, female 6-8 week old BALB/cJRj mice were used. 2×10⁷ cfu challenge dose per animal was applied intravenously. Passive immunization with antibodies was performed by intraperitoneal injection of mAbs diluted in 500 μl in PBS 24 h prior to the lethal, intravenous challenge by bacteria. Typically 100 μg mAb/animal (˜5 mg/kg) dose was used in three independent experiments with 5 mice/group (total 15 mice/mAb treatment). Control groups received an isotype-matched (IgG1) irrelevant mAb. Statistical analysis was performed by analysis of survival curves by the log-rank (Mantel-Cox) test using GraphPad Prism 5.04 Software.

All the anti-SpA mAbs tested provided significant protection in this model (<p=0.01) (FIG. 15).

### REFERENCES

Falugi F, Kim H K, Missiakas D M, Schneewind O. Role of Protein A in the evasion of host adaptive immune responses by Staphylococcus aureus. MBio. 2013 Aug. 27; 4(5):e00575-13. doi: 10.1128/mBio.00575-13.

Smith E, Visai L, Kerrigan S, Speziale P, Foster T. The Sbi protein is a multifunctional immune evasion factor of Staphylococcus aureus. Infect Immun 2011 79,3801-9.

Sasso E, Silverman G, Mannik M. Human IgA and IgG F(ab′)2 that bind to staphylococcal Protein A belong to the VHIII subgroup. J. Immunol 1991 147,1877-83.

Goodyear C S, Silverman G J. B cell superantigens: a microbe's answer to innate-like B cells and natural antibodies. Springer Semin Immunopathol. 2005 March; 26(4):463-84. Review.

Goodyear C S, Silverman G J. Staphylococcal toxin induced preferential and prolonged in vivo deletion of innate-like B lymphocytes. Proc Natl Acad Sci USA. 2004 Aug. 3; 101(31):11392-7. Epub 2004 Jul. 23.#

Gómez M I, Lee A, Reddy B, Muir A, Soong G, Pitt A, Cheung A, Prince A. Staphylococcus aureus Protein A induces airway epithelial inflammatory responses by activating TNFR1. Nat Med. 2004 August; 10(8):842-8. Epub 2004 Jul. 11.

Gómez M I, O'Seaghdha M, Magargee M, Foster T J, Prince A S. Staphylococcus aureus protein A activates TNFR1 signaling through conserved IgG binding domains. J Biol Chem. 2006 Jul. 21; 281(29):20190-6. Epub 2006 May 18.

Gómez M I, Seaghdha M O, Prince A S. Staphylococcus aureus Protein A activates TACE through EGFR-dependent signaling. EMBO J. 2007 Feb. 7; 26(3):701-9. Epub 2007 Jan. 25.

Hartleib J, Köhler N, Dickinson R B, Chhatwal G S, Sixma J J, Hartford O M, Foster T J, Peters G, Kehrel B E, Herrmann M. Protein A is the von Willebrand factor binding protein on Staphylococcus aureus. Blood. 2000 Sep. 15; 96(6):2149-56.

O'Seaghdha M, van Schooten C J, Kerrigan S W, Emsley J, Silverman G J, Cox D, Lenting P J, Foster T J. Staphylococcus aureus Protein A binding to von Willebrand factor A1 domain is mediated by conserved IgG binding regions. FEBS J. 2006 November; 273(21):4831-41. Epub 2006 Sep. 25.

Jonsson P, Lindberg M, Haraldsson I, Wadstrom T. Virulence of Staphylococcus aureus in a mouse mastitis model: Studies of alpha hemolysin, coagulase, and protein a as possible virulence determinants with protoplast fusion and gene cloning. Infect Immun. 1985; 49(3):765-769.

Patel A H, Nowlan P, Weavers E D, Foster T. Virulence of Protein A-deficient and alpha-toxin deficient mutants of Staphylococcus aureus isolated by allele replacement. Infect Immun. 1987; 55(12):3103-3110.

Cheng A G, Kim H K, Burts M L, Krausz T, Schneewind O, Missiakas D M. Genetic requirements for Staphylococcus aureus abscess formation and persistence in host tissues. FASEB J. 2009; 23(10):3393-3404.

Pauli N T, Kim H K, Falugi F, Huang M, Dulac J, Henry Dunand C, Zheng N Y, Kaur K, Andrews S F, Huang Y, DeDent A, Frank K M, Charnot-Katsikas A, Schneewind O, Wilson P C. Staphylococcus aureus infection induces Protein A-mediated immune evasion in humans. J Exp Med. 2014 Nov. 17; 211(12):2331-9. doi: 10.1084/jem.20141404. Epub 2014 Oct. 27.

Kim H K, Cheng A G, Kim H Y, Missiakas D M, Schneewind O. Nontoxigenic protein A vaccine for methicillin-resistant Staphylococcus aureus infections in mice. J Exp Med. 2010 Aug. 30; 207(9):1863-70. Epub 2010 Aug. 16.

Kim H K, Falugi F, Thomer L, Missiakas D M, Schneewind O. Protein A suppresses immune responses during Staphylococcus aureus bloodstream infection in guinea pigs. MBio. 2015 Jan. 6; 6(1). pii: e02369-14. doi: 10.1128/mBio.02369-14.

Kim H K, Emolo C, DeDent A C, Falugi F, Missiakas D M, Schneewind O. Protein A-specific monoclonal antibodies and prevention of Staphylococcus aureus disease in mice. Infect Immun. 2012 October;80(10):3460-70. doi: 10.1128/IAI.00230-12. Epub 2012 Jul. 23.

Kim H K, Emolo C, Missiakas D, Schneewind O. A monoclonal antibody that recognizes the E domain of staphylococcal Protein A. Vaccine. 2014 Jan. 16; 32(4):464-9. doi: 10.1016/j.vaccine.2013.11.054. Epub 2013 Nov. 27.

Thammavongsa V, Rauch S, Kim H K, Missiakas D M, Schneewind O. Protein A-neutralizing monoclonal antibody protects neonatal mice against Staphylococcus aureus. Vaccine. 2015 Jan. 15; 33(4):523-6. doi: 10.1016/j.vaccine.2014.11.051. Epub 2014 Dec. 6.

Rouha H et al. Five birds, one stone: Neutralization of a-hemolysin and 4 bi-component leukocidins of Staphylococcus aureus with a single human monoclonal antibody. mAbs 2015 7,243254.

1-17. (canceled)

18. A monoclonal antibody that counteracts Staphylococcus aureus, comprising a CDR binding site that specifically recognizes wild-type SpA-E of Staphylococcus aureus, and is cross-specific and further recognizes at least SpA-A and SpA-D of Staphylococcus aureus, and which is any of
a) an antibody comprising an antibody heavy chain variable domain (VH) and an antibody light chain variable domain (VL), the antibody comprising six complementarity determining regions (CDR), CDR1 to CDR6, wherein the VH comprises CDR1, CDR2 and CDR3, and the VL comprises CDR4, CDR5 and CDR6, wherein the CDR1 to CDR6 sequences are any one of the antibody sequences listed in Table 1, or
b) a functionally active variant of an antibody of a) (parent antibody), the functionally active variant comprising one or more point mutations in any of the CDR1 to CDR6 sequences of said parent antibody thereby obtaining a CDR variant, which CDR variant has a sequence identity of at least 60% with the CDR sequence of the parent antibody,
wherein said antibody binds with wild-type and mutant SpA with a dissociation constant ratio K_{D} (mutant SpA)/K_{D} (wild-type SpA) of at least 0.5, as determined by binding to wild-type SpA-D and mutant SpA-D, wherein said wild-type SpA-D consists of the amino acid sequence identified as SEQ ID 138, and said mutant SpA-D consists of the amino acid sequence identified as SEQ ID 143.

19. The antibody of claim 18, wherein the CDR binding site further recognizes at least one of SpA-B, SpA-C, Sbi-I, and Sbi-II.

20. The antibody of claim 18, wherein the antibody recognizes at least three immunoglobulin-binding protein (IGBP) domains.

21. The antibody of claim 18, wherein the antibody recognizes at least SpA-E, SpA-A and SpA-D.

22. The antibody of claim 18, wherein the antibody competes with SpA and optionally Sbi binding to IgG-Fc.

23. The antibody of claim 18, wherein the antibody is a full-length monoclonal antibody, an antibody fragment thereof comprising at least the CDR binding site, or a fusion protein comprising at least the CDR binding site.

24. The antibody of claim 18, wherein the functionally active variant is a human, humanized, chimeric, murine and/or affinity matured variant of the parent antibody.

25. The antibody of claim 18, wherein the functionally active variant comprises one or more point mutations in any of the CDR sequences of the parent antibody.

26. The antibody of claim 18, wherein the antibody is:
i)
A1) an antibody comprising:
a) a CDR1 consisting of the amino acid sequence of SEQ ID 43; and
b) a CDR2 consisting of the amino acid sequence of SEQ ID 44; and
c) a CDR3 consisting of the amino acid sequence of SEQ ID 45; and
d) a CDR4 consisting of the amino acid sequence of SEQ ID 103; and
e) a CDRS consisting of the amino acid sequence of SEQ ID 104; and
f) a CDR6 consisting of the amino acid sequence of SEQ ID 105; or
B1) an antibody that is a functionally active variant of the antibody of A1, which has at least 60% sequence identity with each of the CDR sequences; or
ii)
A2) an antibody comprising:
a) a CDR1 consisting of the amino acid sequence of SEQ ID 13; and
b) a CDR2 consisting of the amino acid sequence of SEQ ID 14; and
c) a CDR3 consisting of the amino acid sequence of SEQ ID 15; and
d) a CDR4 consisting of the amino acid sequence of SEQ ID 73; and
e) a CDR5 consisting of the amino acid sequence of SEQ ID 74; and
f) a CDR6 consisting of the amino acid sequence of SEQ ID 75; or
B2) an antibody that is a functionally active variant of the antibody of A2 which has at least 60% sequence identity with each of the CDR sequences.

27. A monoclonal antibody that counteracts Staphylococcus aureus, comprising a CDR binding site that specifically recognizes wild-type SpA-E of Staphylococcus aureus, is cross-specific and further recognizes at least SpA-A and SpA-D of Staphylococcus aureus, wherein the antibody comprises an antibody heavy chain variable domain (VH) and an antibody light chain variable domain (VL), the antibody comprising six complementarity determining regions (CDR), CDR1 to CDR6, wherein the VH comprises CDR1, CDR2 and CDR3, and the VL comprises CDR4, CDR5 and CDR6, wherein the antibody is:
i)
A1) an antibody comprising:
a) a CDR1 consisting of the amino acid sequence of SEQ ID 43; and
b) a CDR2 consisting of the amino acid sequence of SEQ ID 44; and
c) a CDR3 consisting of the amino acid sequence of SEQ ID 45; and
d) a CDR4 consisting of the amino acid sequence of SEQ ID 103; and
e) a CDR5 consisting of the amino acid sequence of SEQ ID 104; and
f) a CDR6 consisting of the amino acid sequence of SEQ ID 105; or
B1) an antibody that is a functionally active variant of the antibody of A1 which has at least 60% sequence identity with each of the CDR sequences; or
ii)
A2) an antibody comprising
a) a CDR1 consisting of the amino acid sequence of SEQ ID 13; and
b) a CDR2 consisting of the amino acid sequence of SEQ ID 14; and
c) a CDR3 consisting of the amino acid sequence of SEQ ID 15; and
d) a CDR4 consisting of the amino acid sequence of SEQ ID 73; and
e) a CDR5 consisting of the amino acid sequence of SEQ ID 74; and
f) a CDR6 consisting of the amino acid sequence of SEQ ID 75; or
B2) an antibody that is a functionally active variant of the antibody of A2 which has at least 60% sequence identity with each of the CDR sequences.

28. The antibody of claim 18, wherein the antibody comprises any one of the heavy chain sequences SEQ ID 152-162, and the light chain sequence SEQ ID 163.

29. A method of treating a subject at risk of or suffering from S. aureus infection or colonization comprising administering to the subject an effective amount of an antibody to limit the infection in the subject or to ameliorate a disease condition resulting from said infection or to inhibit S. aureus disease pathogenesisn, wherein the antibody is a monoclonal antibody that counteracts Staphylococcus aureus, comprising a CDR binding site that specifically recognizes wild-type SpA-E of Staphylococcus aureus, and is cross-specific and further recognizes at least SpA-A and SpA-D of Staphylococcus aureus, and which is any of
a) an antibody comprising an antibody heavy chain variable domain (VH) and an antibody light chain variable domain (VL), the antibody comprising six complementarity determining regions (CDR), CDR1 to CDR6, wherein the VH comprises CDR1, CDR2 and CDR3, and the VL comprises CDR4, CDR5 and CDR6, wherein the CDR1 to CDR6 sequences are any one of the antibody sequences listed in Table 1, or
b) a functionally active variant of an antibody of a) (parent antibody), the functionally active variant comprising one or more point mutations in any of the CDR1 to CDR6 sequences of said parent antibody thereby obtaining a CDR variant, which CDR variant has a sequence identity of at least 60% with the CDR sequence of the parent antibody,
wherein said antibody binds with wild-type and mutant SpA with a dissociation constant ratio K_{D} (mutant SpA)/K_{D} (wild-type SpA) of at least 0.5, as determined by binding to wild-type SpA-D and mutant SpA-D, wherein said wild-type SpA-D consists of the amino acid sequence identified as SEQ ID 138, and said mutant SpA-D consists of the amino acid sequence identified as SEQ ID 143.

30. A pharmaceutical preparation comprising the antibody of claim 18 and a pharmaceutically acceptable carrier or excipient.

31. The pharmaceutical preparation of claim 30, wherein the pharmaceutical preparation comprises a parenteral or mucosal formulation.

32. A diagnostic preparation comprising the antibody of claim 18, optionally containing the antibody with a label and/or a further diagnostic reagent with a label.

33. A method of detecting a S. aureus infection comprising contacting the diagnostic preparation of claim 32 with a biological sample, and determining a S. aureus infection from a specific immune reaction of the antibody.

34. An isolated nucleic acid molecule encoding an antibody of claim 18.

35. A method of producing an antibody of claim 18, wherein a host cell comprising a coding sequence to express the antibody is cultivated or maintained under conditions to produce said antibody.
